(19) United States
(12) Patent Application Publication
(10) Pub. No.: US 2025/0243162 A1
(43) Pub. Date: Jul. 31, 2025

(54) COMPOUND FOR ORGANIC ELECTRIC ELEMENT, ORGANIC ELECTRIC ELEMENT USING THE SAME, AND ELECTRONIC DEVICE THEREOF

(71) Applicant: DUK SAN NEOLUX CO., LTD., Cheonan-si (KR)

(72) Inventors: Jun Seok SEO, Cheonan-si (KR); Junggeun LEE, Cheonan-si (KR); Tae Bin CHA, Cheonan-si (KR); Sun-Hee LEE, Cheonan-si (KR)

(73) Assignee: DUK SAN NEOLUX CO., LTD., Cheonan-si (KR)

(21) Appl. No.: 19/078,977

(22) Filed: Mar. 13, 2025

Related U.S. Application Data
(63) Continuation-in-part of application No. 18/912,870, filed on Oct. 11, 2024.
Application No. 18/912,870 is a division of application No. 17/230,061, filed on Apr. 14, 2021, now Pat. No. 12,178,118.
Application No. 17/230,061 is a continuation of application No. 17/212,776, filed on Mar. 25, 2021, now Pat. No. 12,041,847.
Application No. 17/212,776 is a continuation of application No. 16/344,684, filed on Apr. 24, 2019, now Pat. No. 11,910,705.

(30) Foreign Application Priority Data
Feb. 2, 2018 (KR) 10-2018-0013219

(51) Int. Cl. C07D 209/82; C07D 209/86; C07D 333/76; C07D 403/10; C07D 403/14; C07D 405/14; C07D 409/14; C09K 11/06; H10K 50/11; H10K 50/15; H10K 50/18; H10K 50/85; H10K 71/00; H10K 71/16; H10K 85/60; H10K 101/00; H10K 101/10; H10K 101/40; H10K 102/00

(52) U.S. Cl. CPC C07D 209/82; C07D 209/86; C07D 333/76; C07D 403/10; C07D 403/14; C07D 405/14; C07D 409/14; C09K 11/06; H10K 50/18; H10K 85/626; H10K 85/633; H10K 85/636; H10K 85/654; H10K 85/6572; H10K 85/6574; H10K 85/6576; C09K 2211/1018; H10K 50/11; H10K 50/15; H10K 50/181; H10K 50/85; H10K 71/00; H10K 71/164; H10K 2101/10; H10K 2101/40; H10K 2101/90; H10K 2102/351

(57) ABSTRACT

Provided are a compound of Formula 32 capable of improving luminous efficiency, stability and lifetime of an organic electric element employing the same, the organic electric element and an electronic device thereof.

### CROSS-REFERENCE TO RELATED APPLICATIONS

This application is a continuation-in-part of U.S. patent application Ser. No. 18/912,870, filed on Oct. 11, 2024, which is a divisional of U.S. patent application Ser. No. 17/230,061, filed on Apr. 14, 2021, now U.S. Pat. No. 12,178,118, issued on Dec. 24, 2024, which is a continuation of U.S. patent application Ser. No. 17/212,776, filed on Mar. 25, 2021, now U.S. Pat. No. 12,041,847, issued on Jul. 16, 2024, which is a continuation of U.S. patent application Ser. No. 16/344,684, filed on Apr. 24, 2019, now U.S. Pat. No. 11,910,705, issued on Feb. 20, 2024, which is a 371 of international PCT/KR2019/000678, filed on Jan. 17, 2019, which claims the benefit of priority from Korean Patent Application No. 10-2018-0013219 filed on Feb. 2, 2018, the contents of each of which are incorporated herein by reference in its entirety.

### BACKGROUND

### Technical Field

The present invention relates to compound for organic electric element, organic electric element using the same, and an electronic device thereof.

### Background Art

In general, organic light emitting phenomenon refers to a phenomenon that converts electric energy into light energy by using an organic material. An organic electric element using an organic light emitting phenomenon usually has a structure including an anode, a cathode, and an organic material layer interposed therebetween. Here, in order to increase the efficiency and stability of the organic electronic element, the organic material layer is often composed of a multi-layered structure composed of different materials, and for example, may include a hole injection layer, a hole transport layer, an emitting layer, an electron transport layer, an electron injection layer and the like.

A material used as an organic material layer in an organic electric element may be classified into a light emitting material and a charge transport material, such as a hole injection material, a hole transport material, an electron transport material, an electron injection material and the like depending on its function.

And the light emitting material may be classified into a polymer type and a low molecular type depending on the molecular weight, and into a fluorescent material derived from the singlet excited state of electrons and a phosphorescent material derived from the triplet excited state of electrons depending on the light emitting mechanism. Further, the light emitting material can be classified into blue, green, and red light emitting materials and yellow and orange light emitting materials necessary for realizing better natural color depending on the luminescent color.

Meanwhile, when only one material is used as a light emitting material, there arises a problem that the maximum light emission wavelength shifts to a long wavelength due to intermolecular interaction, the color purity drops, or the efficiency of the device decreases due to the light emission attenuation effect, therefore a host/dopant system can be used as a light emitting material in order to increase luminous efficiency through increase of color purity and energy transfer. When the small amount of dopant having a smaller energy band gap than the host forming the emitting layer is mixed on the emitting layer, the excitons generated in the emitting layer are transported to the dopant to emit light with high efficiency. At this time, since the wavelength of the host is shifted to the wavelength band of the dopant, light of a desired wavelength can be obtained depending on the type of the dopant used.

Currently, the portable display market is growing in size as a large-area display, which requires more power than the power consumption required by existing portable displays. Therefore, power consumption is a very important factor for portable displays, which have a limited power source, such as a battery, and efficiency and lifetime issues must be solved.

In order to solve problems of power consumption, luminous efficiency, and lifespan, research on a Tandem OLED comprising 2 or more stacks (or light emitting units) in which each organic material layer includes an emitting layer is being conducted. In particular, research is being conducted to improve the power consumption, luminous efficiency, and lifespan of organic electronic elements by improving organic materials included in the stack.

Efficiency, lifespan and driving voltage are related to each other, and when the efficiency is increased, the driving voltage is relatively decreased, and as the driving voltage is decreased, crystallization of organic materials due to Joule heating generated during driving decreases, and consequently, the lifespan tends to increase. However, the efficiency cannot be maximized simply by improving the organic material. This is because, when the energy level and T1 value between each organic material, and the intrinsic properties (mobility, interfacial properties, etc.) of materials are optimally combined, long lifespan and high efficiency can be achieved at the same time. Therefore, it is necessary to develop a material that has high thermal stability and can efficiently balance charge in the emitting layer.

Also, a charge generation layer is required to increase the efficiency of current generated in the emitting layer and facilitate charge distribution between 2 or more stacks (or light emitting units) in Tandem OLED. Depending on how smooth the charge distribution in the charge generation layer is, it affects the driving voltage of the entire device, and depending on the energy level difference between the n-type charge generation layer and the p-type charge generation layer, the concentration of the doping material doped in the charge generation layer, and the like, the charge injection characteristics and lifespan of the device are also affected.

That is, the efficiency, lifespan, and driving voltage of the organic electronic element may vary depending on which organic material is used in combination with which layer in Tandem OLED, but until now, development of stable and efficient organic material layer materials for organic electronic elements have not been sufficiently developed.

As a precedent reference, U.S. Pat. No. 8,334,058 B2 is referred to.

### DETAILED DESCRIPTION OF THE INVENTION

### Summary

Using the characteristics of the polycyclic compound, the present invention provides a compound capable of maximizing the effect of improving luminous efficiency and long life, while maintaining or slightly reducing the driving voltage of the device, and an organic electric element using the same and an electronic device thereof.

### Technical Solution

The present invention provides compounds represented by Formula (32), organic electric elements comprising the same and electronic devices thereof.

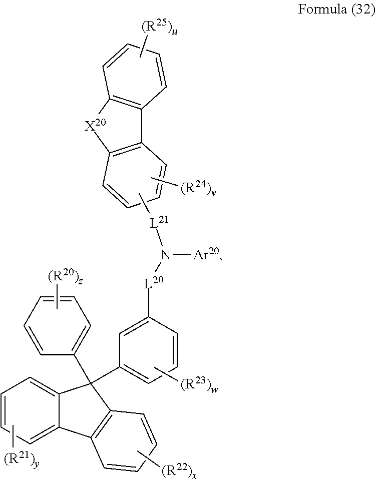

### Effects of the Invention

By using the compound according to the present invention, it is possible to achieve a high luminous efficiency, a low driving voltage, and a high heat resistance of the element, and can greatly improve the color purity and lifetime of the element.

### BRIEF DESCRIPTION OF THE DRAWINGS

FIGS. 1 to 3 each illustrates an example of an organic electric element according to the present invention.

| | |
|---|---|
| 100, 200, 300: organic electronic | 110: the first electrode |
| element | |
| 120: hole injection layer | 130: hole transport layer |
| 140: emitting layer | 150: electron transport layer |
| 160: electron injection layer | 170: second electrode |
| 180: light efficiency enchancing Layer | 210: buffer layer |
| 220: emitting auxiliary layer | 320: first hole injection layer |
| 330: first hole transport layer | 340: first emitting layer |
| 350: first electron transport layer | 360: first charge generation layer |
| 361: second charge generation layer | 420: second hole injection layer |
| 430: second hold transport layer | 440: second emitting layer |
| 450: second electron transport layer | CGL: charge generation layer |
| ST1: first stack | ST2: second stack |

### DETAILED DESCRIPTION

Hereinafter, some embodiments of the present invention will be described in detail. Further, in the following description of the present invention, a detailed description of known functions and configurations incorporated herein will be omitted when it may make the subject matter of the present invention rather unclear.

In addition, terms, such as first, second, A, B, (a), (b) or the like may be used herein when describing components of the present invention. Each of these terminologies is not used to define an essence, order or sequence of a corresponding component but used merely to distinguish the corresponding component from other component(s). It should be noted that if a component is described as being “connected”, “coupled”, or “connected” to another component, the component may be directly connected or connected to the other component, but another component may be “connected “,” coupled” or “connected” between each component.

As used in the specification and the accompanying claims, unless otherwise stated, the following is the meaning of the term as follows.

Unless otherwise stated, the term “halo” or “halogen”, as used herein, includes fluorine, bromine, chlorine, or iodine.

Unless otherwise stated, the term “alkyl” or “alkyl group”, as used herein, has a single bond of 1 to 60 carbon atoms, and means saturated aliphatic functional radicals including a linear alkyl group, a branched chain alkyl group, a cycloalkyl group (alicyclic), an cycloalkyl group substituted with a alkyl or an alkyl group substituted with a cycloalkyl.

Unless otherwise stated, the term “haloalkyl” or “halogen alkyl”, as used herein, includes an alkyl group substituted with a halogen.

Unless otherwise stated, the term “heteroalkyl”, as used herein, means alkyl substituted one or more of carbon atoms consisting of an alkyl with heteroatom.

Unless otherwise stated, the term “alkenyl” or “alkynyl”, as used herein, has double or triple bonds of 2 to 60 carbon atoms, but is not limited thereto, and includes a linear or a branched chain group.

Unless otherwise stated, the term “cycloalkyl”, as used herein, means alkyl forming a ring having 3 to 60 carbon atoms, but is not limited thereto.

Unless otherwise stated, the term “alkoxyl group”, “alkoxy group” or “alkyloxy group”, as used herein, means an oxygen radical attached to an alkyl group, but is not limited thereto, and has 1 to 60 carbon atoms.

Unless otherwise stated, the term “alkenoxyl group”, “alkenoxy group”, “alkenyloxyl group” or “alkenyloxy group”, as used herein, means an oxygen radical attached to an alkenyl group, but is not limited thereto, and has 2 to 60 carbon atoms.

Unless otherwise stated, the term “aryloxyl group” or “aryloxy group”, as used herein, means an oxygen radical attached to an aryl group, but is not limited thereto, and has 6 to 60 carbon atoms.

Unless otherwise stated, the term “aryl group” or “arylene group”, as used herein, has 6 to 60 carbon atoms, but is not limited thereto. Herein, the aryl group or arylene group means a monocyclic and polycyclic aromatic group, and may also be formed in conjunction with an adjacent group. Examples of “aryl group” may include a phenyl group, a biphenyl group, a fluorene group, or a spirofluorene group.

The prefix “aryl” or “ar” means a radical substituted with an aryl group. For example, an arylalkyl may be an alkyl substituted with an aryl, and an arylalenyl may be an alkenyl substituted with aryl, and a radical substituted with an aryl has a number of carbon atoms as defined herein.

Also, when prefixes are named subsequently, it means that substituents are listed in the order described first. For example, an arylalkoxy means an alkoxy substituted with an aryl, an alkoxylcarbonyl means a carbonyl substituted with an alkoxyl, and an arylcarbonylalkenyl also means an alkenyl substituted with an arylcarbonyl, wherein the arylcarbonyl may be a carbonyl substituted with an aryl.

Unless otherwise stated, the term “heteroalkyl”, as used herein, means alkyl including one or more of heteroatoms. Unless otherwise stated, the term “heteroaryl group” or “heteroarylene group”, as used herein, means a C2 to C60 aryl including one or more of heteroatoms or arylene group, but is not limited thereto, and includes at least one of monocyclic and polycyclic rings, and may also be formed in conjunction with an adjacent group.

Unless otherwise stated, the term “heterocyclic group”, as used herein, contains one or more heteroatoms, but is not limited thereto, has 2 to 60 carbon atoms, includes any one of monocyclic and polycyclic rings, and may include heteroaliphadic ring and/or heteroaromatic ring. Also, the heterocyclic group may also be formed in conjunction with an adjacent group.

Unless otherwise stated, the term “heteroatom”, as used herein, represents at least one of N, O, S, P, or Si.

Also, the term “heterocyclic group” may include a ring including SO₂ instead of carbon consisting of cycle. For example, “heterocyclic group” includes compound below.

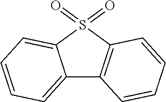 [SMILES: O=S1(=O)c2ccccc2-c2ccccc21]

Unless otherwise stated, the term “aliphatic”, as used herein, means an aliphatic hydrocarbon having 1 to 60 carbon atoms, and the term “aliphatic ring”, as used herein, means an aliphatic hydrocarbon ring having 3 to 60 carbon atoms.

Unless otherwise stated, the term “ring”, as used herein, means an aliphatic ring having 3 to 60 carbon atoms, or an aromatic ring having 6 to 60 carbon atoms, or a hetero ring having 2 to 60 carbon atoms, or a fused ring formed by the combination of them, and includes a saturated or unsaturated ring.

Other hetero compounds or hetero radicals other than the above-mentioned hetero compounds include, but are not limited thereto, one or more heteroatoms.

Unless otherwise stated, the term “carbonyl”, as used herein, is represented by —COR′, wherein R′ may be hydrogen, an alkyl having 1 to 20 carbon atoms, an aryl having 6 to 30 carbon atoms, a cycloalkyl having 3 to 30 carbon atoms, an alkenyl having 2 to 20 carbon atoms, an alkynyl having 2 to 20 carbon atoms, or the combination of these.

Unless otherwise stated, the term “ether”, as used herein, is represented by —R—O—R′, wherein R or R′ may be independently hydrogen, an alkyl having 1 to 20 carbon atoms, an aryl having 6 to 30 carbon atoms, a cycloalkyl having 3 to 30 carbon atoms, an alkenyl having 2 to 20 carbon atoms, an alkynyl having 2 to 20 carbon atoms, or the combination of these.

Unless otherwise stated, the term “substituted or unsubstituted”, as used herein, means that substitution is substituted by at least one substituent selected from the group consisting of, but is not limited thereto, deuterium, halogen, an amino group, a nitrile group, a nitro group, a C₁-C₂₀ alkyl group, a C₁-C₂₀ alkoxyl group, a C₁-C₂₀ alkylamine group, a C₁-C₂₀ alkylthiopen group, a C₆-C₂₀ arylthiopen group, a C₂-C₂₀ alkenyl group, a C₂-C₂₀ alkynyl group, a C₃-C₂₀ cycloalkyl group, a C₆-C₂₀ aryl group, a C₆-C₂₀ aryl group substituted by deuterium, a C₈-C₂₀ arylalkenyl group, a silane group, a boron group, a germanium group, and a C₂-C₂₀ heterocyclic group.

Unless otherwise expressly stated, the Formula used in the present invention, as used herein, is applied in the same manner as the substituent definition according to the definition of the exponent of the following Formula.

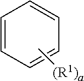

Wherein, when a is an integer of zero, it means the substituent R¹ is absent. That is, when a is 0, it means that all the carbons forming the benzene ring are bonded to hydrogen. In this case, the sign of the hydrogen bonded to the carbon may be omitted and the formula or compound may be described. When a is an integer of 1, the sole substituent R¹ is linked to any one of the carbon constituting the benzene ring, when a is an integer of 2 or 3, they are respectively bonded as follows, in which R¹ may be the same as or different from each other, and when a is an integer of 4 to 6, and it is bonded to the carbon of the benzene ring in a similar manner, whereas the indication of hydrogen bonded to the carbon forming the benzene ring is omitted.

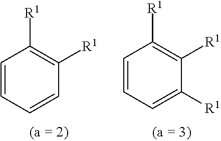

Hereinafter, a layered structure of an organic electronic element including the compound of the present invention will be described with reference to FIG. 1.

In adding reference numerals to the components of each drawing, it should be noted that the same components have the same numerals as much as possible even if they are displayed on different drawings. Also, in describing the present invention, if it is determined that a detailed description of a related known configuration or function may obscure the gist of the present invention, the detailed description will be omitted.

FIG. 1 is an exemplary view of an organic electronic element according to an embodiment of the present invention.

Referring to FIG. 1, an organic electronic element (300) according to an embodiment of the present invention comprises 2 or more sets of multi-layer stacks (ST1, ST2) between a first electrode (110) and a second electrode (170), and a charge generation layer (CGL) may be formed between the stacks of organic material layers.

Specifically, the organic electronic element according to an embodiment of the present invention comprises a first electrode (110), a first stack (ST1), a charge generation layer (CGL), a second stack (ST2), and a second electrode. (170) and a light efficiency enhancing layer (180).

The first stack (ST1) is an organic material layer formed on the first electrode (110), which comprises the first hole injection layer (320), the first hole transport layer (330), the first emitting layer (340), and the first electron transport layer (350), and the second stack (ST2) may comprise a second hole injection layer (420), a second hole transport layer (430), a second emitting layer (440), and a second electron transport layer (450) and if necessary, the second hole injection layer (420) of the second stack (ST2) may be omitted. In this way, the first stack and the second stack may be organic material layers having the same stacked structure or organic material layers having different stacked structures.

A charge generation layer (CGL) may be formed between the first stack (ST1) and the second stack (ST2). The charge generation layer (CGL) may comprise a n-type charge generation layer (360) adjacent to the first electron transport layer (350) of the first stack (ST1) and a p-type charge generation layer (361) adjacent to the second hole injection layer (420) of the second stack (ST2). The charge generation layer (CGL) is formed between the first emitting layer (340) and the second emitting layer (440) to increase the efficiency of current generated in each emitting layer and to smoothly distribute charges. In the case of a top emission organic light emitting device, optical energy loss due to SPPs (surface plasmon polaritons) in the second electrode (170) can be reduced by forming the light efficiency enhancing layer (180), and in the case of a bottom emission organic light emitting device, the light efficiency enhancing layer (180) may serve as a buffer for the second electrode (170).

When a plurality of emitting layers are formed by a multi-layer stack structure method as shown in FIG. 1, an organic light emitting device emitting white light by a mixing effect of light emitted from each emitting layer may be manufactured, as well as an organic light emitting device emitting light of various colors. Although not shown in FIG. 1, a buffer layer and an emitting auxiliary layer may be further formed between the hole transport layer and the emitting layer of each stack, and an electron transport auxiliary layer may be further formed between the emitting layer and the electron transport layer.

Although FIG. 1 has described a Tandem OLED with 2 stacks, the organic electronic element according to the present invention can be a Tandem OLED with 2 or more stacks. (e.g, a Tandem OLED with 3 stacks, a Tandem OLED with 4 stacks)

Preferably, the compounds represented by Formula 32 of the present invention may be used as a charge generation layer (CGL), more preferably, the compound represented by Formulas 32 may be used as a p-type charge generation layer.

The p-type charge generation layer may be doped with Formula 3.

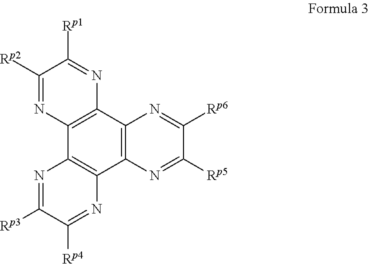

In Formula 3, each symbol may be defined as follows.

1) R^{p1}, R^{p2}, R^{p3}, R^{p4}, R^{p5} and R^{p6} are each independently selected from the group consisting of hydrogen; halogen; nitrile group; nitro group; —SO₂R; —SOR; —SO₂NR²; —SO₃R; trifluoromethyl group; —COOR; —CONHR; —CONRR′; —NRR′; a C₆-C₆₀ aryl group; fluorenyl group; a C₂-C₆₀ heterocyclic group including at least one heteroatom of O, N, S, Si or P; a fused ring group of a C₃-C₆₀ aliphatic ring and a C₆-C₆₀ aromatic ring; a C₁-C₅₀ alkyl group; a C₂-C₂₀ alkenyl group; a C₂-C₂₀ alkynyl group; a C₁-C₃₀ alkoxyl group; and a C₆-C₃₀ aryloxy group;

when R^{p1}, R^{p2}, R^{p3}, R^{p4}, R^{p5} and R^{p6} are an aryl group, it may be preferably a C₆-C₃₀ aryl group, and more preferably a C₆-C₂₅ aryl group, for example, it may be phenyl, biphenyl, terphenyl, naphthalene, etc,

when R^{p1}, R^{p2}, R^{p3}, R^{p4}, R^{p5} and R^{p6} are a heterocyclic group, it may be preferably a C₂-C₃₀ heterocyclic group, and more preferably a C₂-C₂₄ heterocyclic group, for example, it may be pyrazine, thiophene, pyridine, pyrimidoindole, 5-phenyl-5H-pyrimido[5,4-b]indole, quinazoline, benzoquinazoline, carbazole, dibenzoquinazoline, dibenzofuran, dibenzothiophene, benzothienopyrimidine, benzofuropyrimidine, phenothiazine, phenylphenothiazine, etc.,

when R^{p1}, R^{p2}, R^{p3}, R^{p4}, R^{p5} and R^{p6} are a fused ring group, it may be preferably a fused ring group of a C3-Cso aliphatic ring and a C₆-C₃₀ aromatic ring, more preferably a fused ring group of a C₃-C₂₄ aliphatic ring and a C₆-C₂₄ aromatic ring.

when R^{p1}, R^{p2}, R^{p3}, R^{p4}, R^{p5} and R^{p6} are an alkyl group, it may be preferably a C₁-C₃₀ alkyl group, and more preferably a C₁-C₂₄ alkyl group,

when R^{p1}, R^{p2}, R^{p3}, R^{p4}, R^{p5} and R^{p6} are an alkoxyl group, it may be preferably an C₁˜C₂₄ alkoxyl group,

when R^{p1}, R^{p2}, R^{p3}, R^{p4}, R^{p5} and R^{p6} are an aryloxy group, it may be preferably an C₆˜C₂₄ aryloxy group,

2) R and R′ are each independently selected from the group consisting of a C₆-C₆₀ aryl group; fluorenyl group; a C₂-C₆₀ heterocyclic group including at least one heteroatom of O, N, S, Si or P; a fused ring group of a C₃-C₆₀ aliphatic ring and a C₆-C₆₀ aromatic ring; a C₁-C₅₀ alkyl group; a C₂-C₂₀ alkenyl group; and a C₂-C₂₀ alkynyl group;

when R and R′ are an aryl group, it may be preferably a C₆-C₃₀ aryl group, and more preferably a C₆-C₂₅ aryl group, for example, it may be phenyl, biphenyl, terphenyl, naphthalene, etc,

when R and R′ are a heterocyclic group, it may be preferably a C₂-C₃₀ heterocyclic group, and more preferably a C₂-C₂₄ heterocyclic group, for example, it may be pyrazine, thiophene, pyridine, pyrimidoindole, 5-phenyl-5H-pyrimido[5,4-b]indole, quinazoline, benzoquinazoline, carbazole, dibenzoquinazoline, dibenzofuran, dibenzothiophene, benzothienopyrimidine, benzofuropyrimidine, phenothiazine, phenylphenothiazine, etc.,

when R and R′ are a fused ring group, it may be preferably a fused ring group of a C₃-C₃₀ aliphatic ring and a C₆-C₃₀ aromatic ring, more preferably a fused ring group of a C₃-C₂₄ aliphatic ring and a C₆-C₂₄ aromatic ring.

when R and R′ are an alkyl group, it may be preferably a C₁-C₃₀ alkyl group, and more preferably a C₁-C₂₄ alkyl group,

Formula 3 may preferably be any one of the following compounds E-1 to E-5.

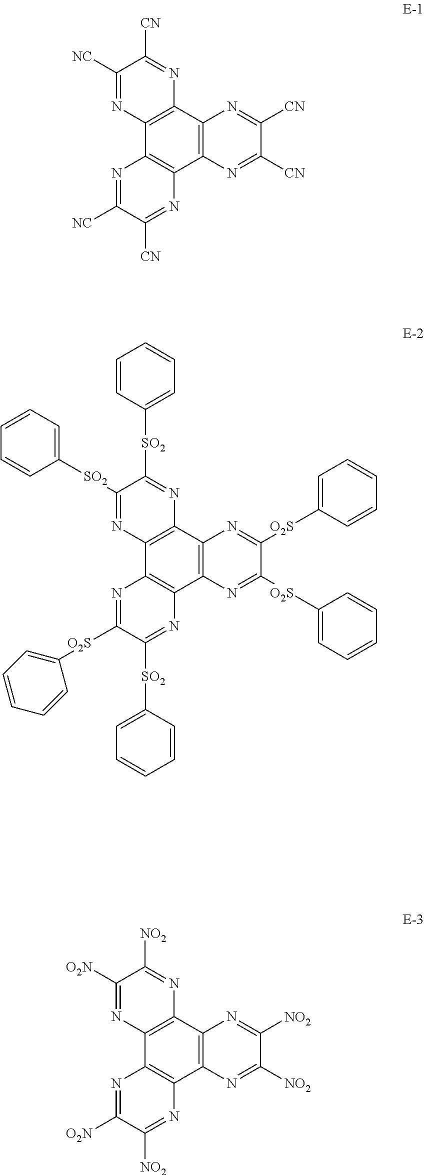 [SMILES: N#Cc1nc2c3nc(C#N)c(C#N)nc3c3nc(C#N)c(C#N)nc3c2nc1C#N ; O=S(=O)(c1ccccc1)c1nc2c3nc(S(=O)(=O)c4ccccc4)c(S(=O)(=O)c4ccccc4)nc3c3nc(S(=O)(=O)c4ccccc4)c(S(=O)(=O)c4ccccc4)nc3c2nc1S(=O)(=O)c1ccccc1 ; O=[N+]([O-])c1nc2c3nc([N+](=O)[O-])c([N+](=O)[O-])nc3c3nc([N+](=O)[O-])c([N+](=O)[O-])nc3c2nc1[N+](=O)[O-]]

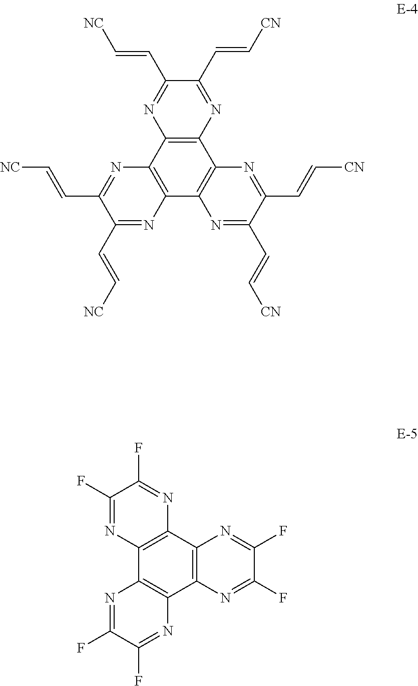 [SMILES: N#C/C=C/c1nc2c3nc(/C=C/C#N)c(/C=C/C#N)nc3c3nc(/C=C/C#N)c(/C=C/C#N)nc3c2nc1/C=C/C#N ; Fc1nc2c3nc(F)c(F)nc3c3nc(F)c(F)nc3c2nc1F]

In another example, the p-type charge generation layer may be doped with any one of the following compounds E-6 to E-26.

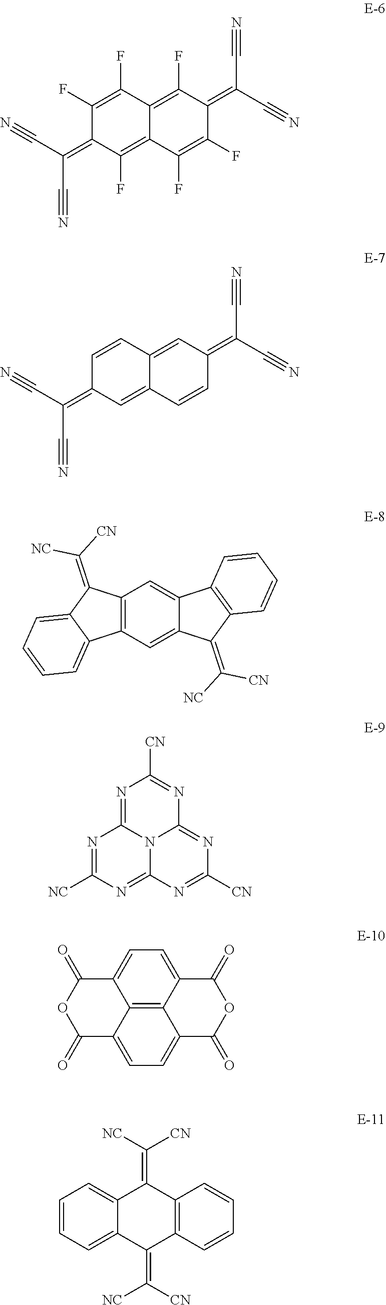 [SMILES: N#CC(C#N)=c1c(F)c(F)c2c(F)c(=C(C#N)C#N)c(F)c(F)c2c1F ; N#CC(C#N)=c1ccc2cc(=C(C#N)C#N)ccc2c1 ; N#CC(C#N)=C1c2ccccc2-c2cc3c(cc21)-c1ccccc1C3=C(C#N)C#N ; N#CC1=NC2=NC(C#N)=NC3=NC(C#N)=NC(=N1)N23 ; O=C1OC(=O)c2ccc3c4c(ccc1c24)C(=O)OC3=O ; N#CC(C#N)=c1c2ccccc2c(=C(C#N)C#N)c2ccccc12]

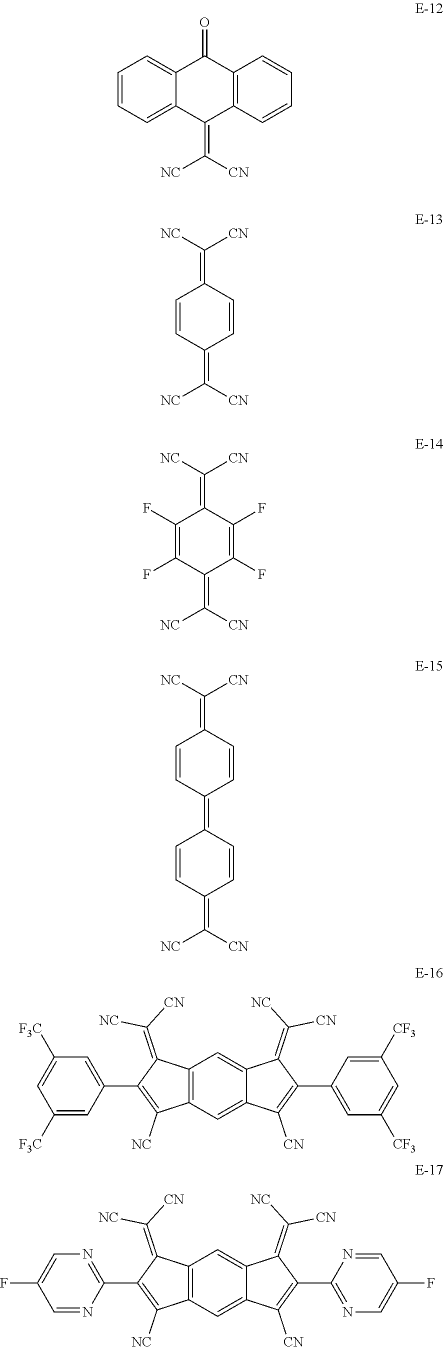 [SMILES: N#CC(C#N)=C1c2ccccc2C(=O)c2ccccc21 ; N#CC(C#N)=c1ccc(=C(C#N)C#N)cc1 ; N#CC(C#N)=c1c(F)c(F)c(=C(C#N)C#N)c(F)c1F ; N#CC(C#N)=c1ccc(=c2ccc(=C(C#N)C#N)cc2)cc1 ; N#CC(C#N)=C1C(c2cc(C(F)(F)F)cc(C(F)(F)F)c2)=C(C#N)c2cc3c(cc21)C(=C(C#N)C#N)C(c1cc(C(F)(F)F)cc(C(F)(F)F)c1)=C3C#N ; N#CC(C#N)=C1C(c2ncc(F)cn2)=C(C#N)c2cc3c(cc21)C(=C(C#N)C#N)C(c1ncc(F)cn1)=C3C#N]

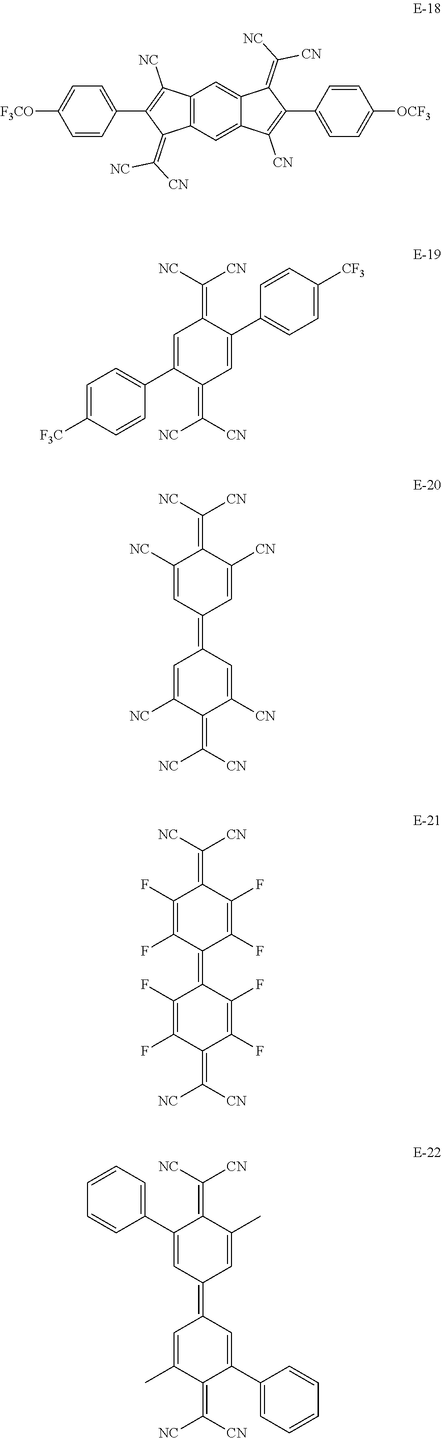 [SMILES: N#CC(C#N)=C1C(c2ccc(OC(F)(F)F)cc2)=C(C#N)c2cc3c(cc21)C(C#N)=C(c1ccc(OC(F)(F)F)cc1)C3=C(C#N)C#N ; N#CC(C#N)=c1cc(-c2ccc(C(F)(F)F)cc2)c(=C(C#N)C#N)cc1-c1ccc(C(F)(F)F)cc1 ; N#CC(C#N)=c1c(C#N)cc(=c2cc(C#N)c(=C(C#N)C#N)c(C#N)c2)cc1C#N ; N#CC(C#N)=c1c(F)c(F)c(=c2c(F)c(F)c(=C(C#N)C#N)c(F)c2F)c(F)c1F ; Cc1c/c(=c2/cc(C)c(=C(C#N)C#N)c(-c3ccccc3)c2)cc(-c2ccccc2)c1=C(C#N)C#N]

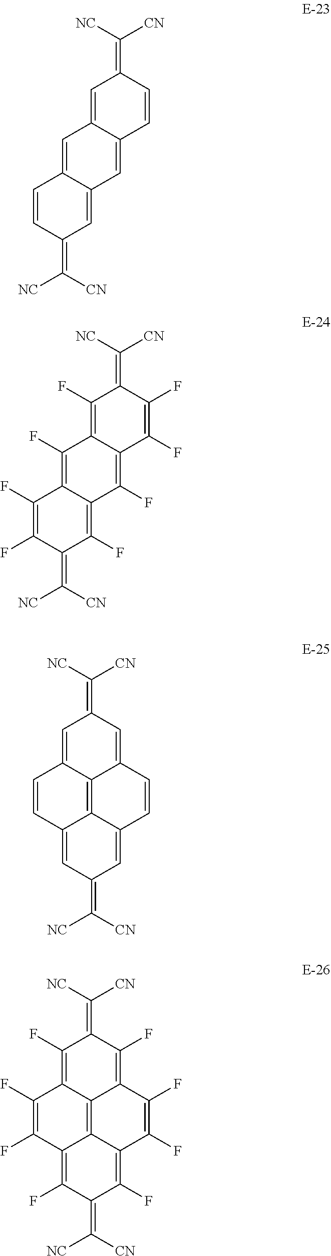

The p-type charge generation layer may be doped with any one of the compounds E-1 to E-18 at 0.1 w % to 50 w %, preferably at 3 w % to 15 w %, and more preferably at 5 w % to 10 w %.

According to a specific example of the present invention, there is provided a compound represented by Formula (32).

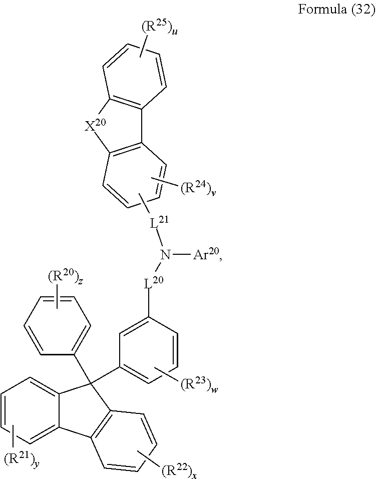

As another example, the present invention provides an organic electronic element comprising an anode; a cathode; an organic material layer formed between the anode and the cathode; wherein the organic material layer includes an emitting layer, an hole transport layer formed between the anode and the emitting layer; an emitting auxiliary layer formed between the emitting layer and the hole transport layer; wherein the hole transport layer or the emitting auxiliary layer comprises a compound represented by Formula (18).

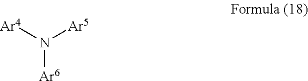

In Formula (18),

Ar⁴ and Ar⁵ are each independently selected from the group consisting of a C₆-C₆₀ aryl group; a fluorenyl group; a C₂-C₆₀ heterocyclic group including at least one heteroatom of O, N, S, Si or P; a fused ring group of a C₃-C₆₀ aliphatic ring and a C₆-C₆₀ aromatic ring; a C₁-C₅₀ alkyl group; a C₂-C₂₀ alkenyl group; a C₂-C₂₀ alkynyl group; a C₁-C₃₀ alkoxyl group; a C₆-C₃₀ aryloxy group; and -L′-N(Rₐ)(R_{b});

wherein, L′ may be selected from the group consisting of a single bond; a C₆-C₆₀ arylene group; a fluorenylene group; a fused ring group of a C₃-C₆₀ aliphatic ring and a C₆-C₆₀ aromatic ring; and a C₂-C₆₀ heterocyclic group;

and the Ra and Rb are each independently selected from the group consisting of a C₆-C₆₀ aryl group; a fluorenyl group; a fused ring group of a C₃-C₆₀ aliphatic ring and a C₆-C₆₀ aromatic ring; and a C₂-C₆₀ heterocyclic group including at least one heteroatom of O, N, S, Si, or P;

or Ar⁴ and Ar⁵ may be bonded to each other to form a ring.

Ar⁶ is selected from the group consisting of a C₆-C₆₀ aryl group; a fluorenyl group; a C₂-C₆₀ heterocyclic group including at least one heteroatom of O, N, S, Si, or P; or is at least one of the following Formulas (1-a), (1-b), (1-c)

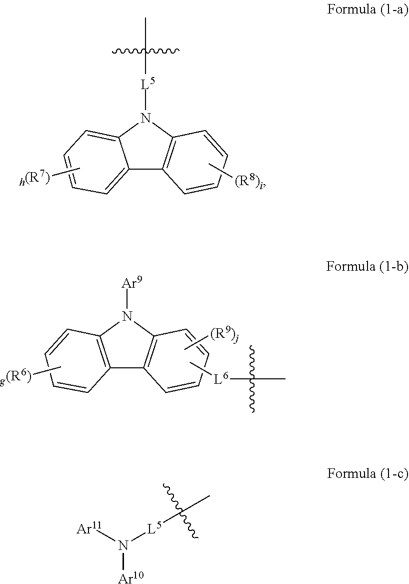

Ar⁹, Ar¹⁰ and Ar¹¹ are each independently selected from the group consisting of a C₆-C₆₀ aryl group; a fluorenyl group; a C₂-C₆₀ heterocyclic group including at least one heteroatom of O, N, S, Si, or P; a fused ring group of a C₃-C₆₀ aliphatic ring and a C₆-C₆₀ aromatic ring; a C₁-C₅₀ alkyl group; a C₂-C₂₀ alkenyl group; a C₂-C₂₀ alkynyl group; a C₁-C₃₀ alkoxyl group; a C6-Cso aryloxy group; and -L′-N(Rₐ)(R_{b});

h, i and g are an integer of 0 to 4; j is an integer of 0 to 3; R⁶, R⁷, R⁸ and R⁹ are the same or different from each other, and are each independently selected from the group consisting of hydrogen; deuterium; halogen; a C₆-C₆₀ aryl group; a fluorenyl group; a C₂-C₆₀ heterocyclic group including at least one heteroatom of O, N, S, Si or P; a fused ring group of a C₃-C₆₀ aliphatic ring and a C₆-C₆₀ aromatic ring; a C₁-C₅₀ alkyl group; a C₂-C₂₀ alkenyl group; a C₂-C₂₀ alkynyl group; a C₁-C₃₀ alkoxyl group; a C₆-C₃₀ aryloxy group; and -L′-N(Rₐ)(R_{b});

wherein in case g, h, i, j are 2 or more, R⁶, R⁷, R⁸ and R⁹ are each in plural being the same or different, and may be bonded to each other to form a ring,

L⁶ is selected from the group consisting of a single bond; a C₆-C₆₀ arylene group; a fluorenylene group; a fused ring group of a C₃-C₆₀ aliphatic ring and a C₆-C₆₀ aromatic ring; and a C₂-C₆₀ heterocyclic group including at least one heteroatom of O, N, S, Si or P;

L⁵ is selected from the group consisting of a C₆-C₆₀ arylene group; a fluorenylene group; a fused ring group of a C₃-C₆₀ aliphatic ring and a C₆-C₆₀ aromatic ring; and a C₂-C₆₀ heterocyclic group including at least one heteroatom of O, N, S, Si or P}

Also, the hole transport layer comprises a compound represented by Formula (19) or Formula (20), and the emitting auxiliary layer comprises a compound represented by Formula (21) or Formula (22)

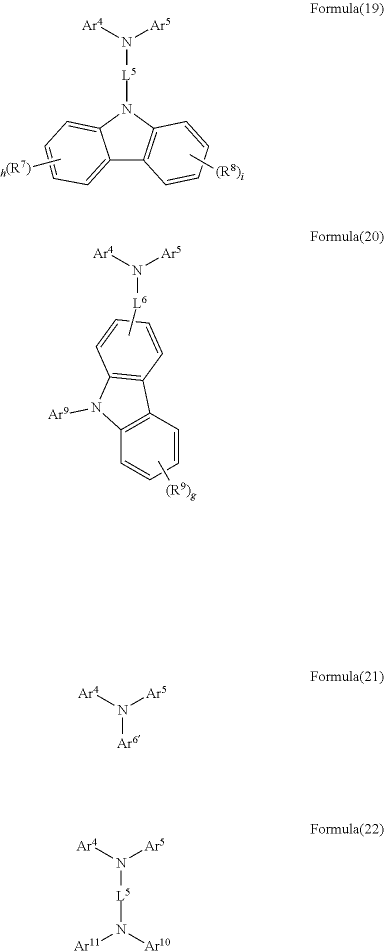

{In Formulas (19) to (22),

1) Ar⁶ is selected from the group consisting of a C₆-C₆₀ aryl group; a fluorenyl group; a C₂-C₆₀ heterocyclic group including at least one heteroatom of O, N, S, Si or P;

2) Ar⁴, Ar⁵, Ar⁹, Ar¹⁰, Ar¹¹, h, i, g, L⁵, L⁶, R⁶, R⁷, R⁸ and R⁹ are the same as defined above.}

Specifically, the compound represented by Formula (18) comprises the following compounds 13-1 to 13-79 and compounds 2-1 to 2-187.

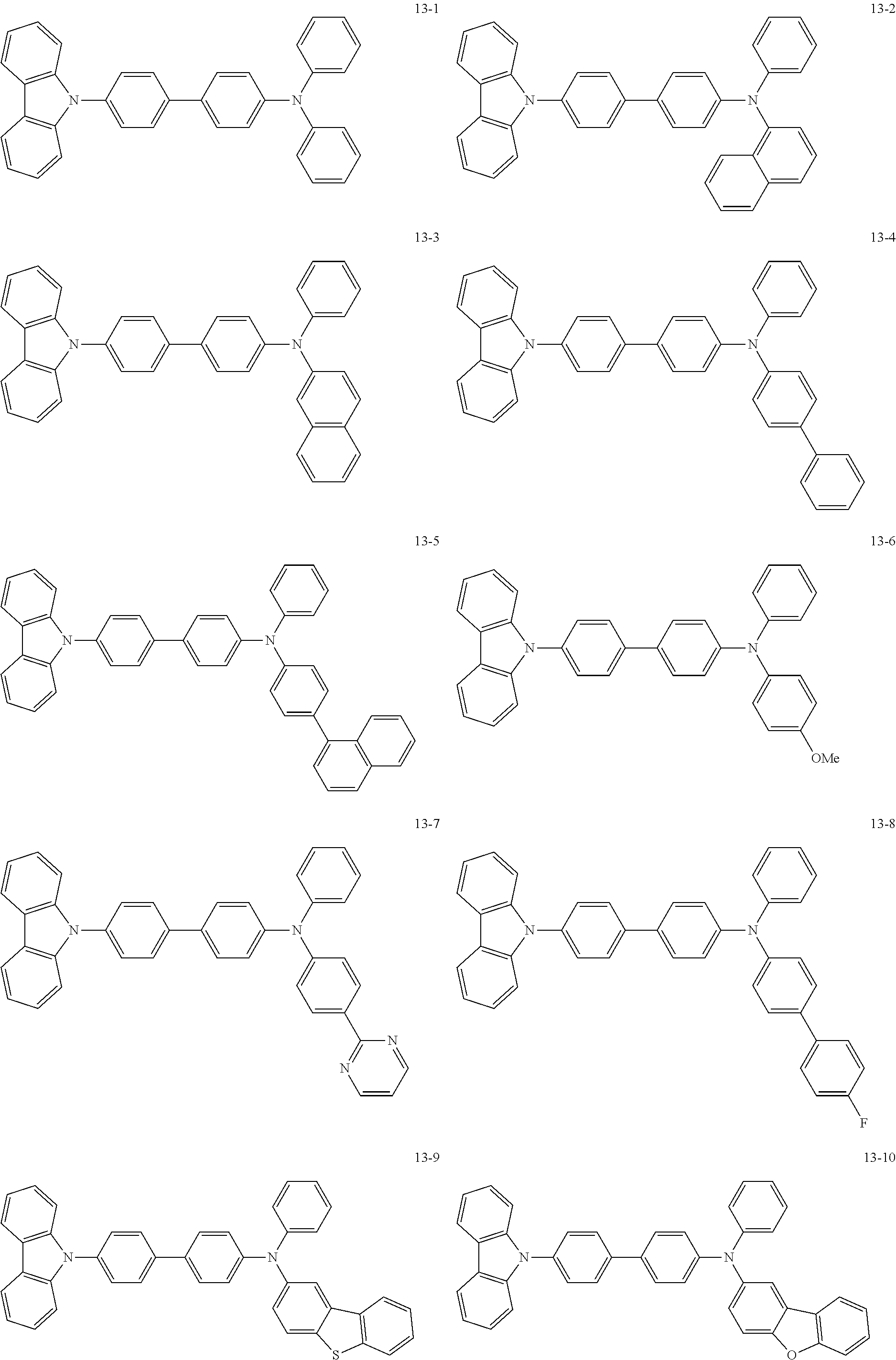

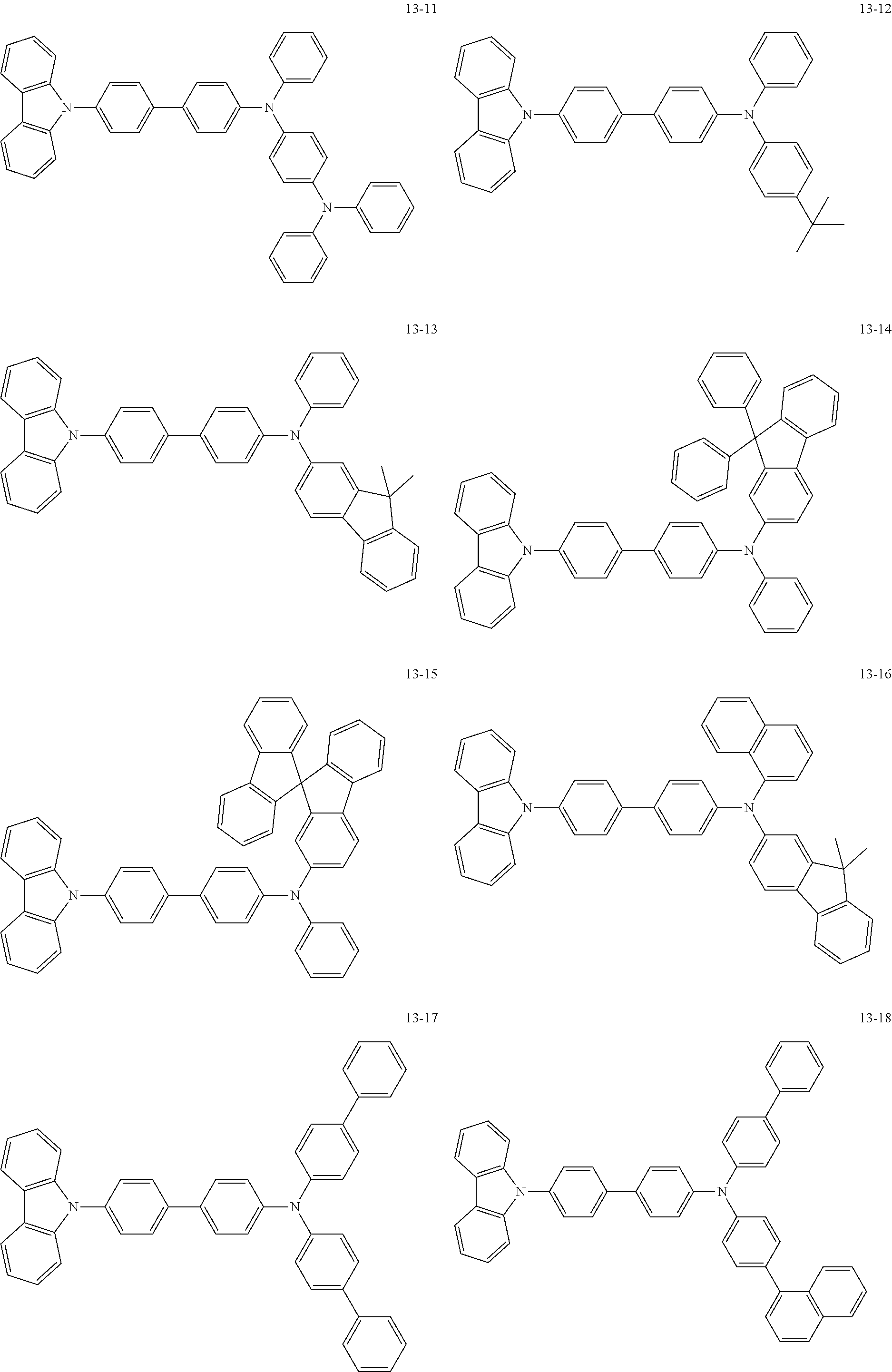

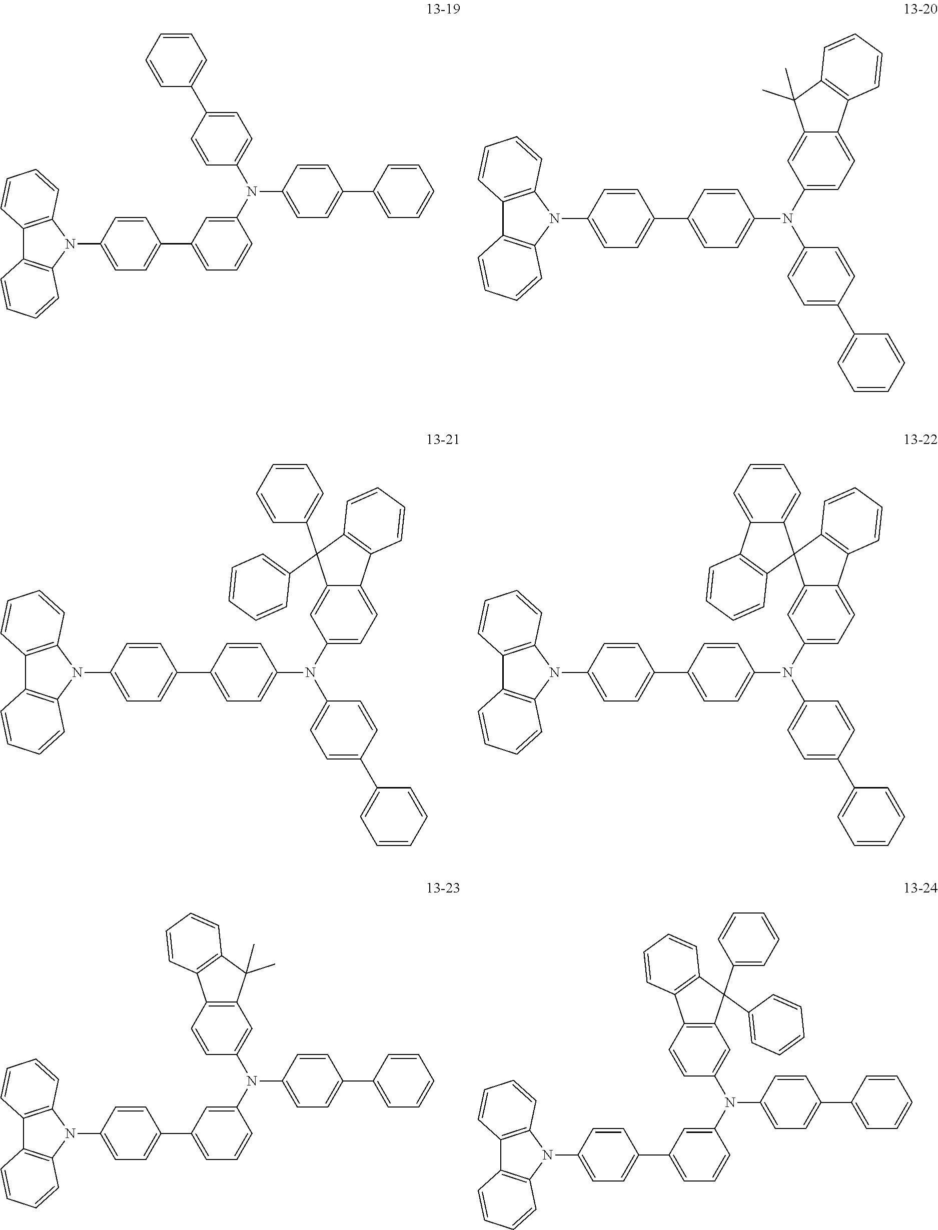

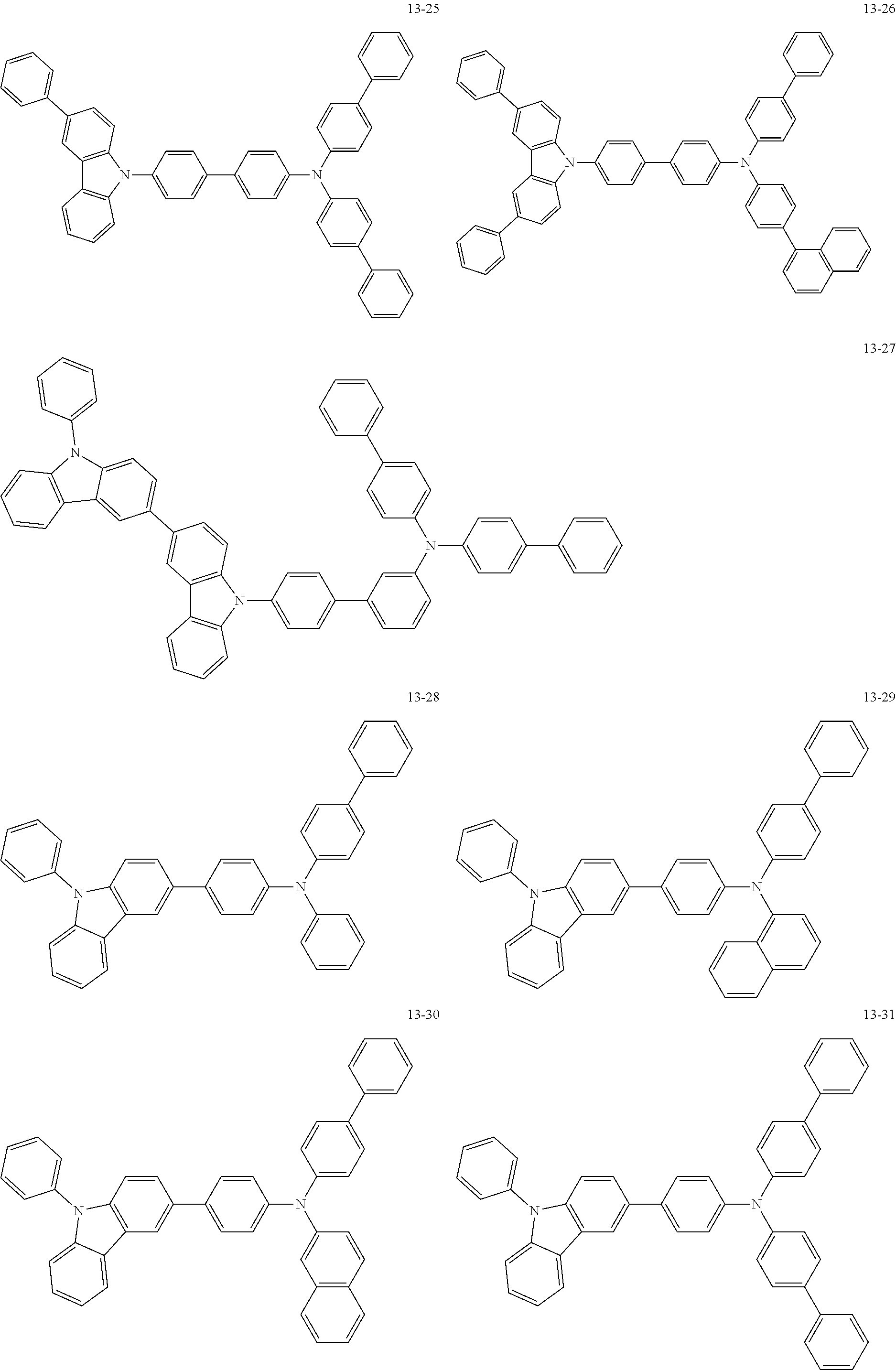

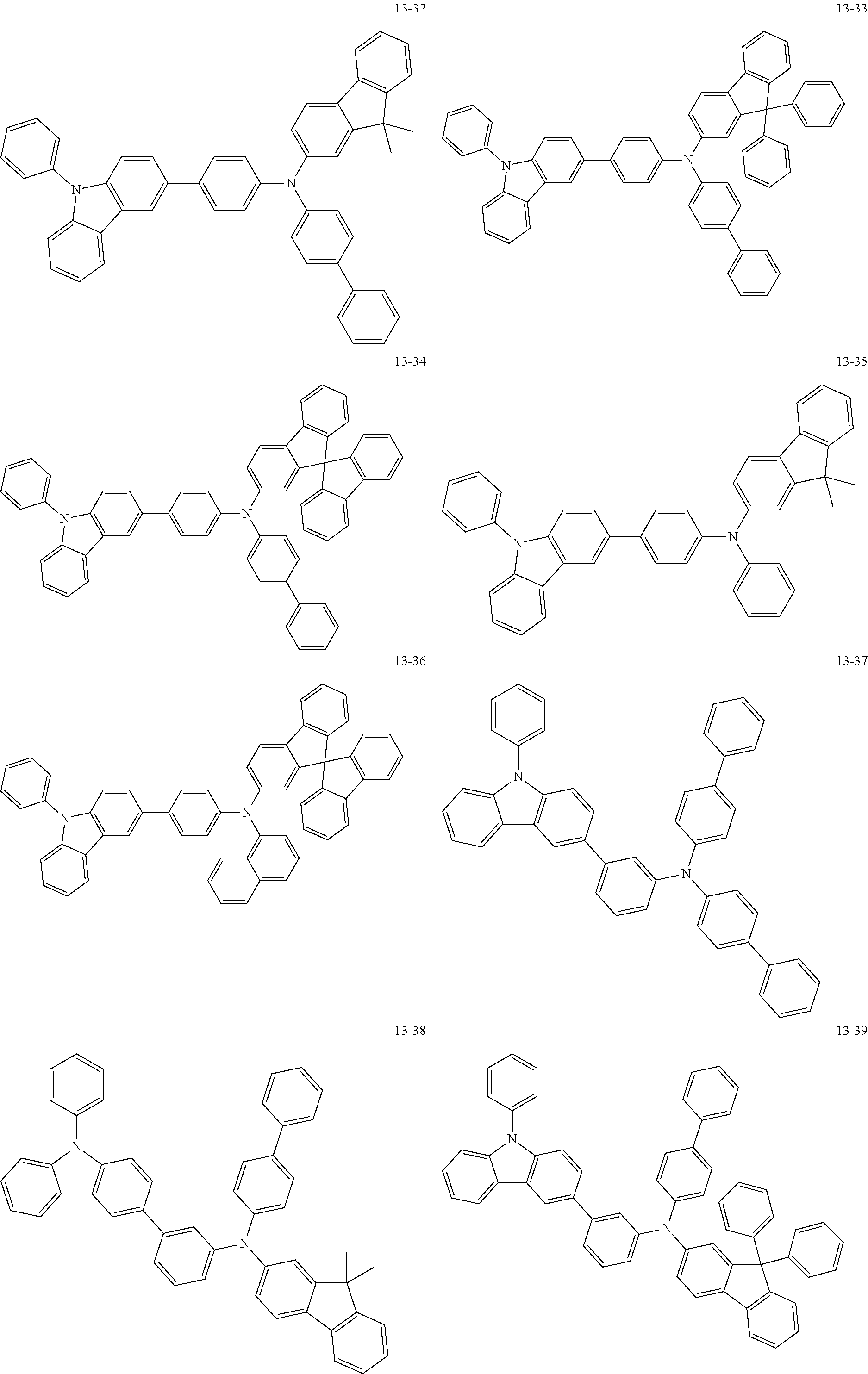

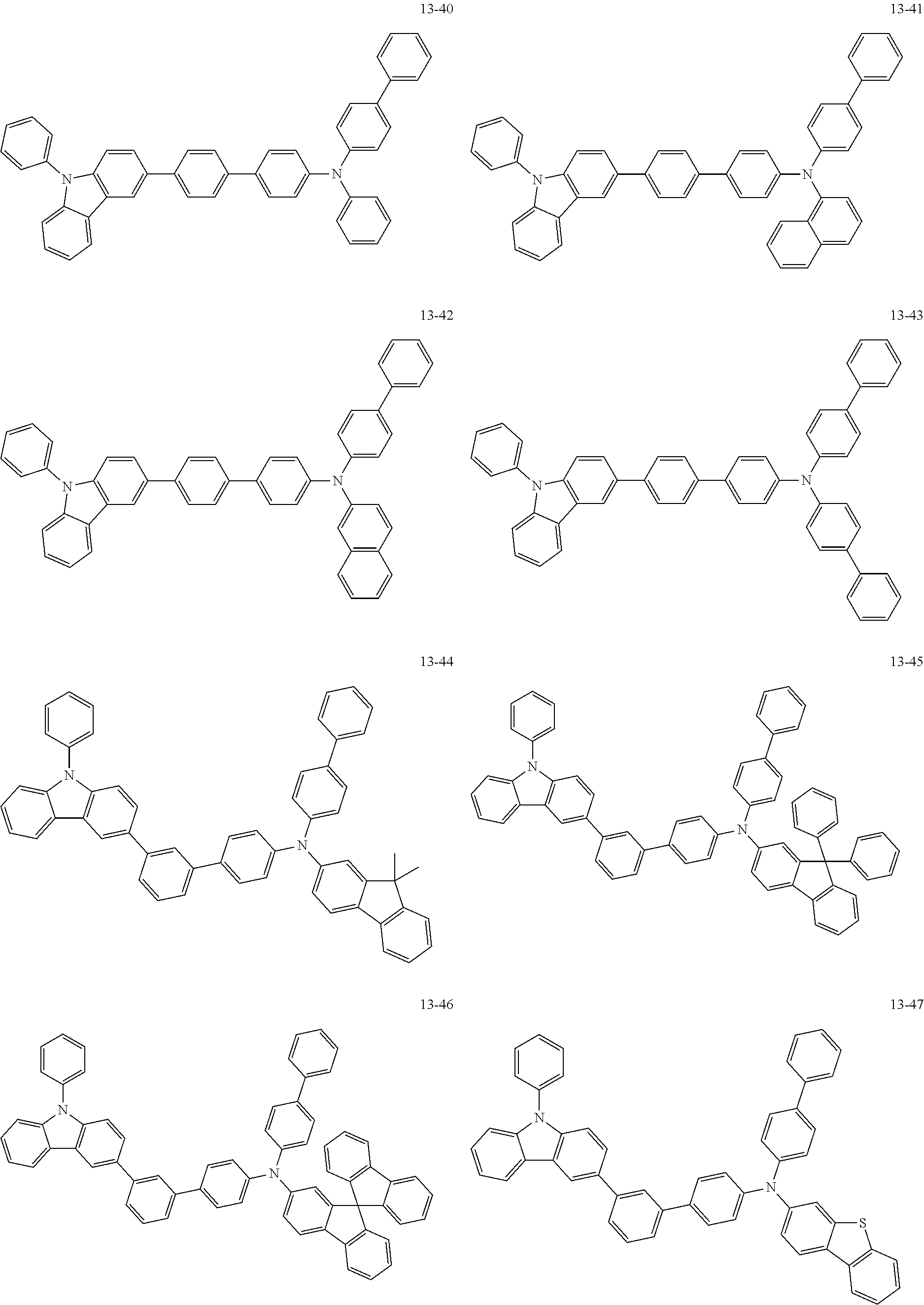

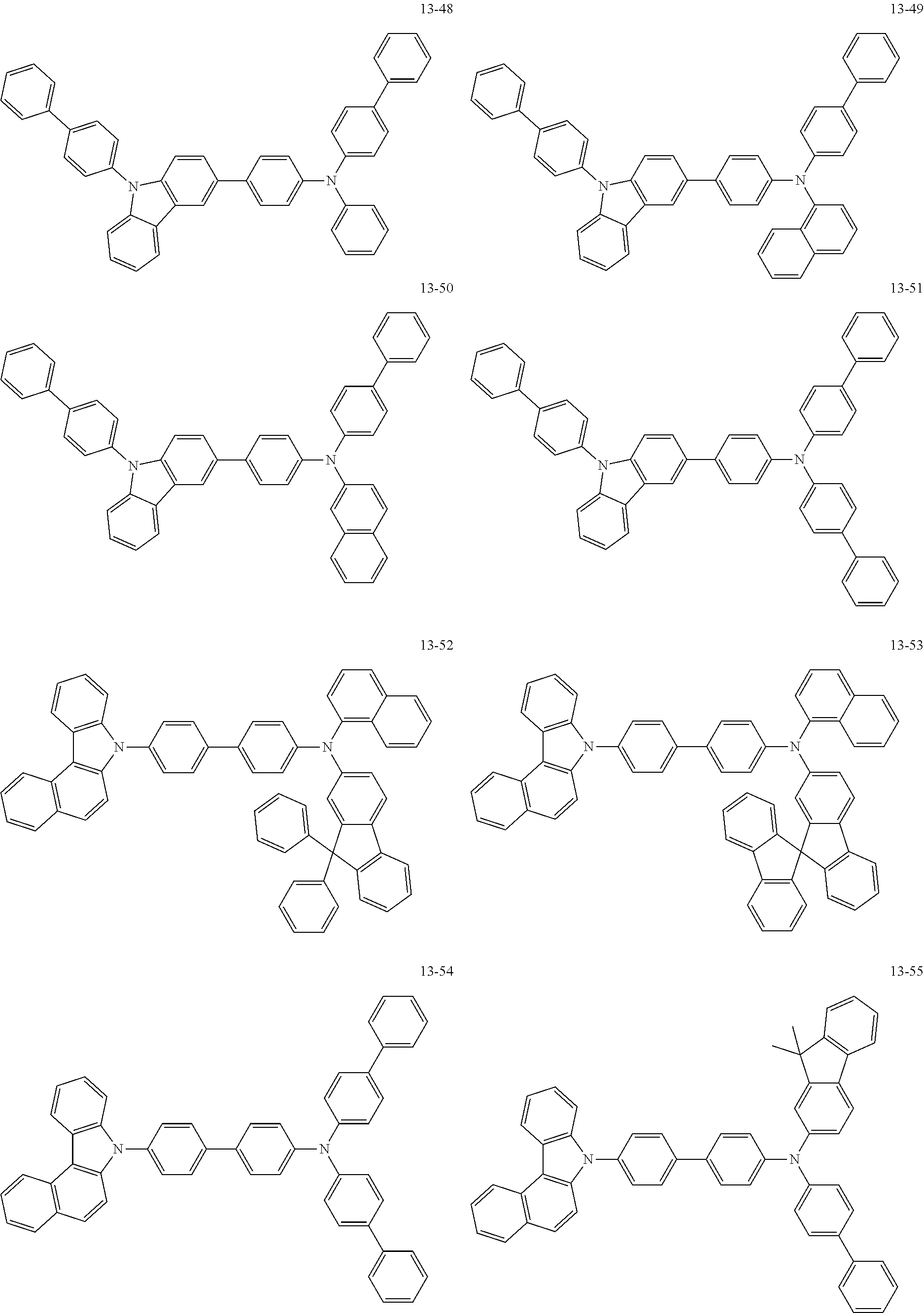

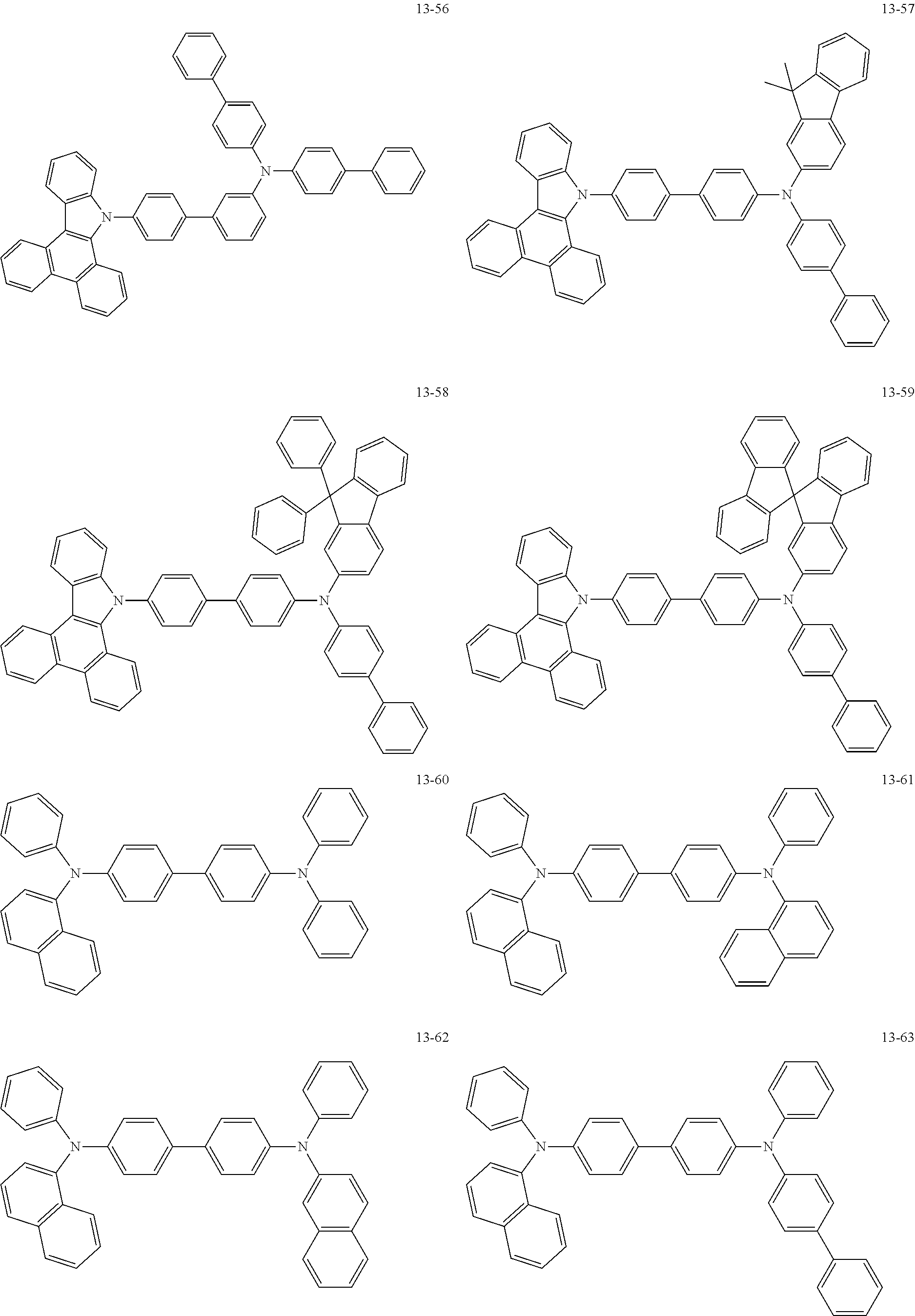

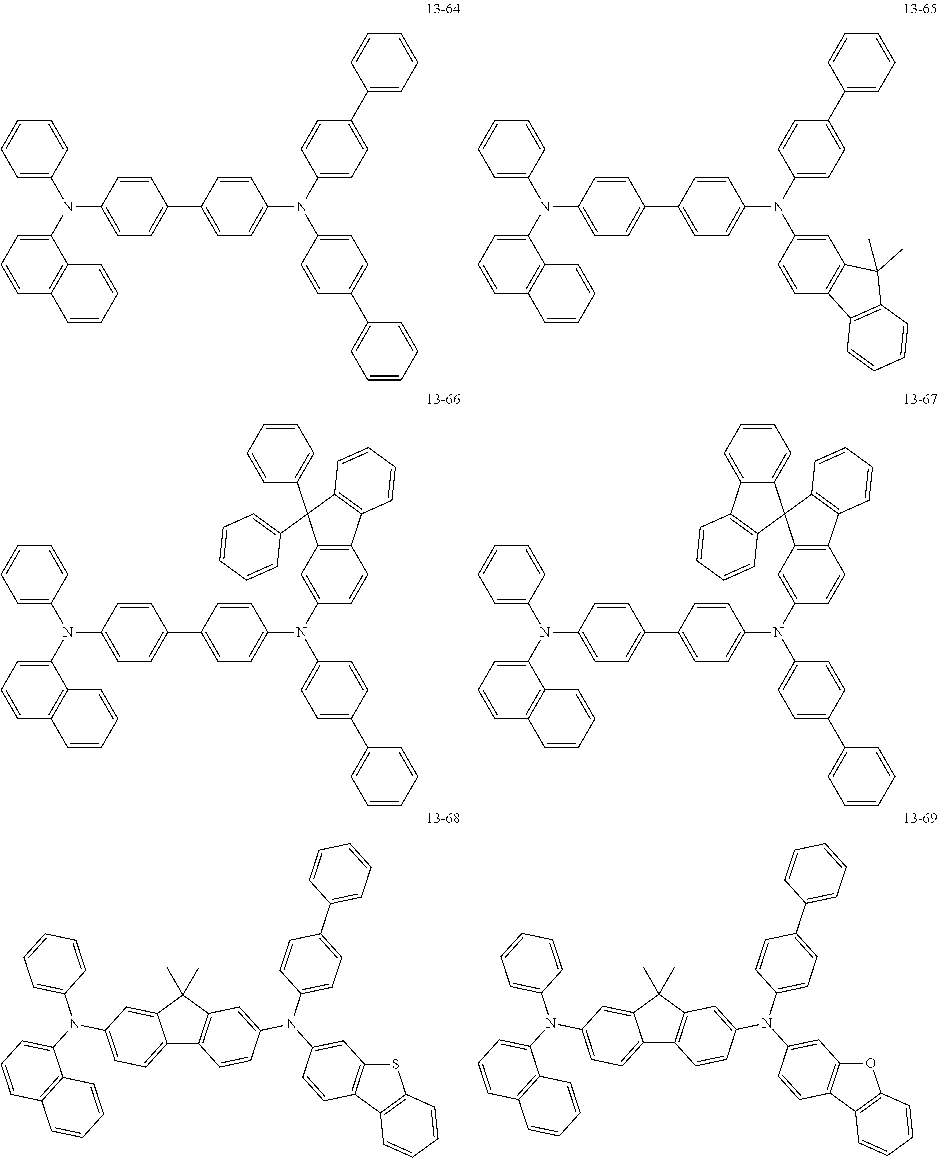

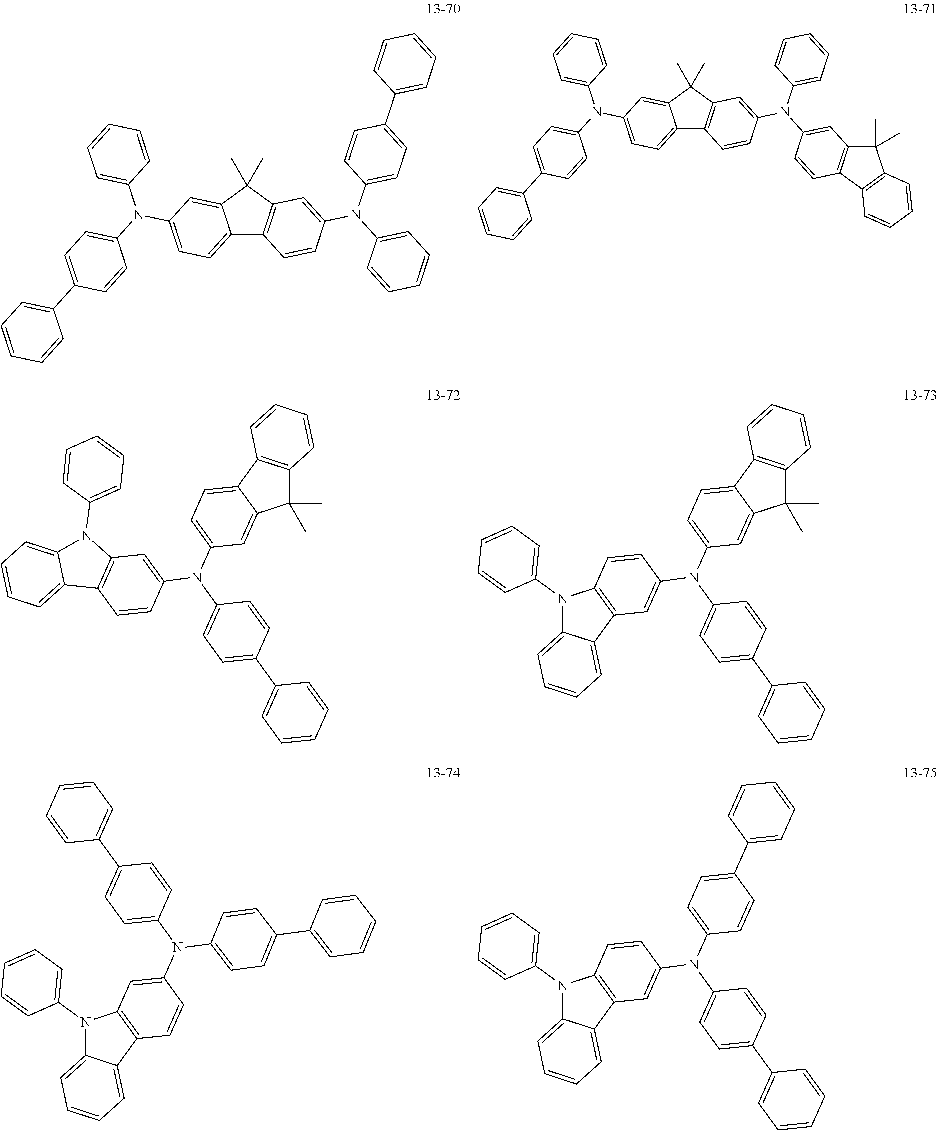

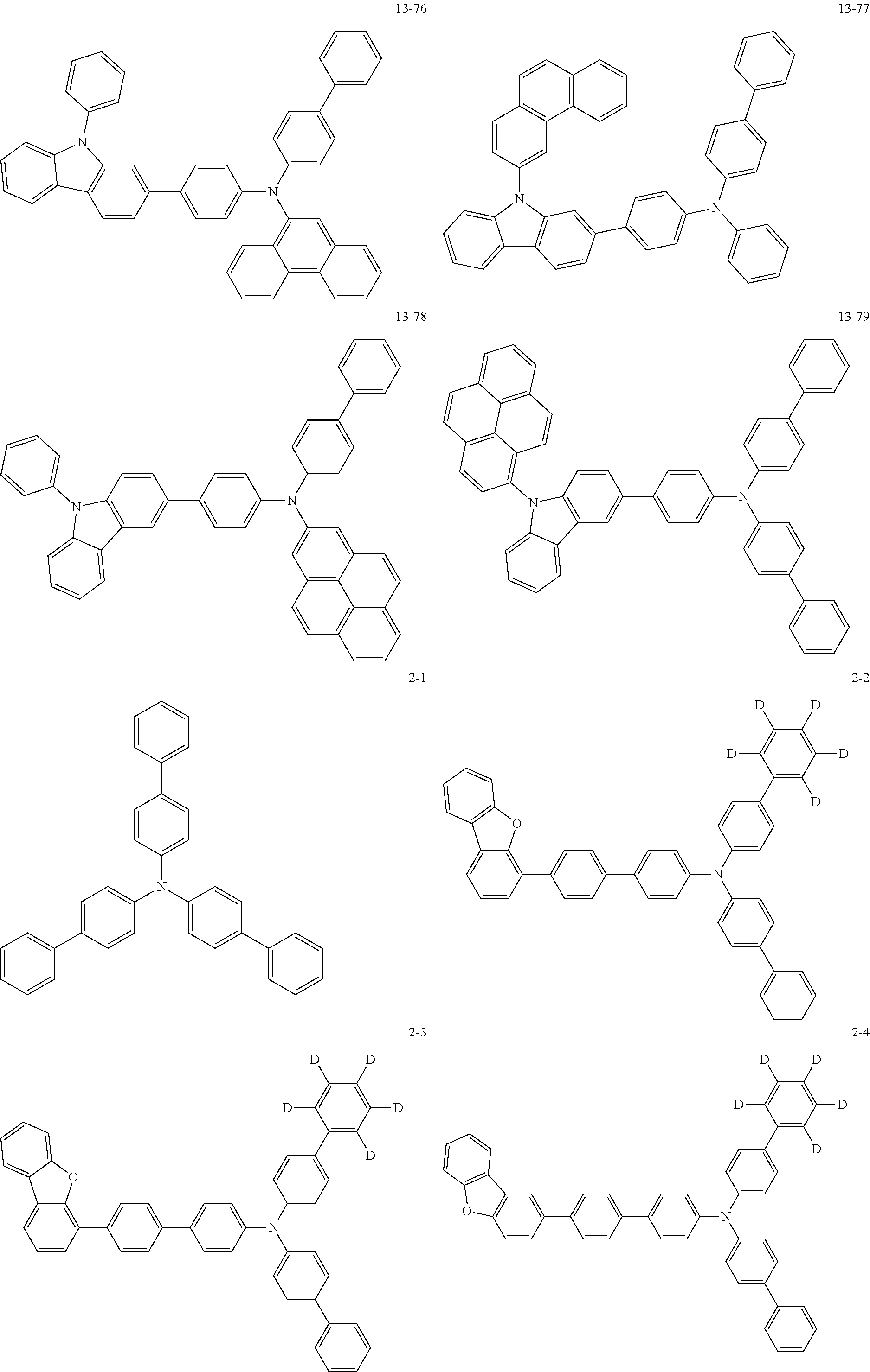

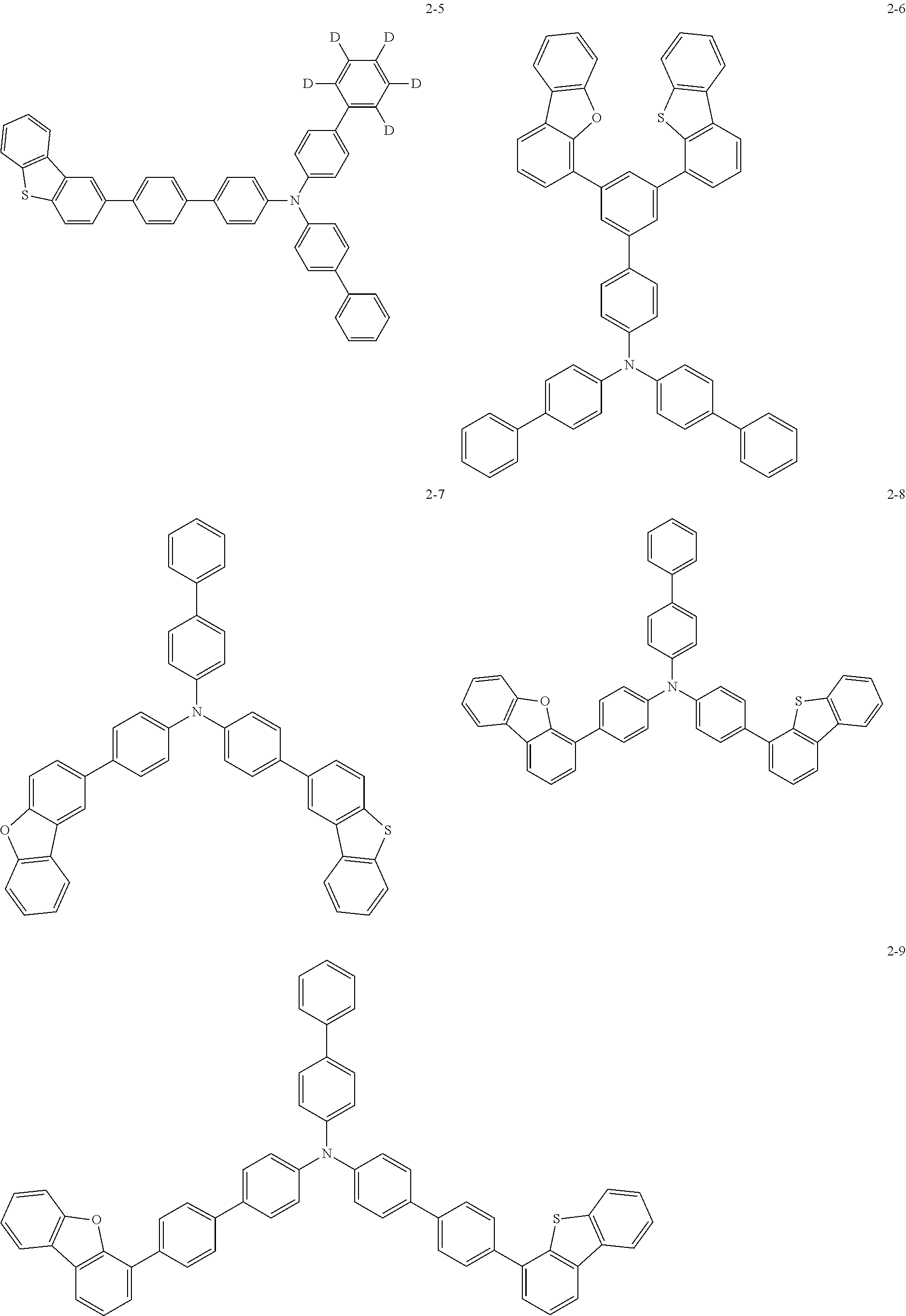

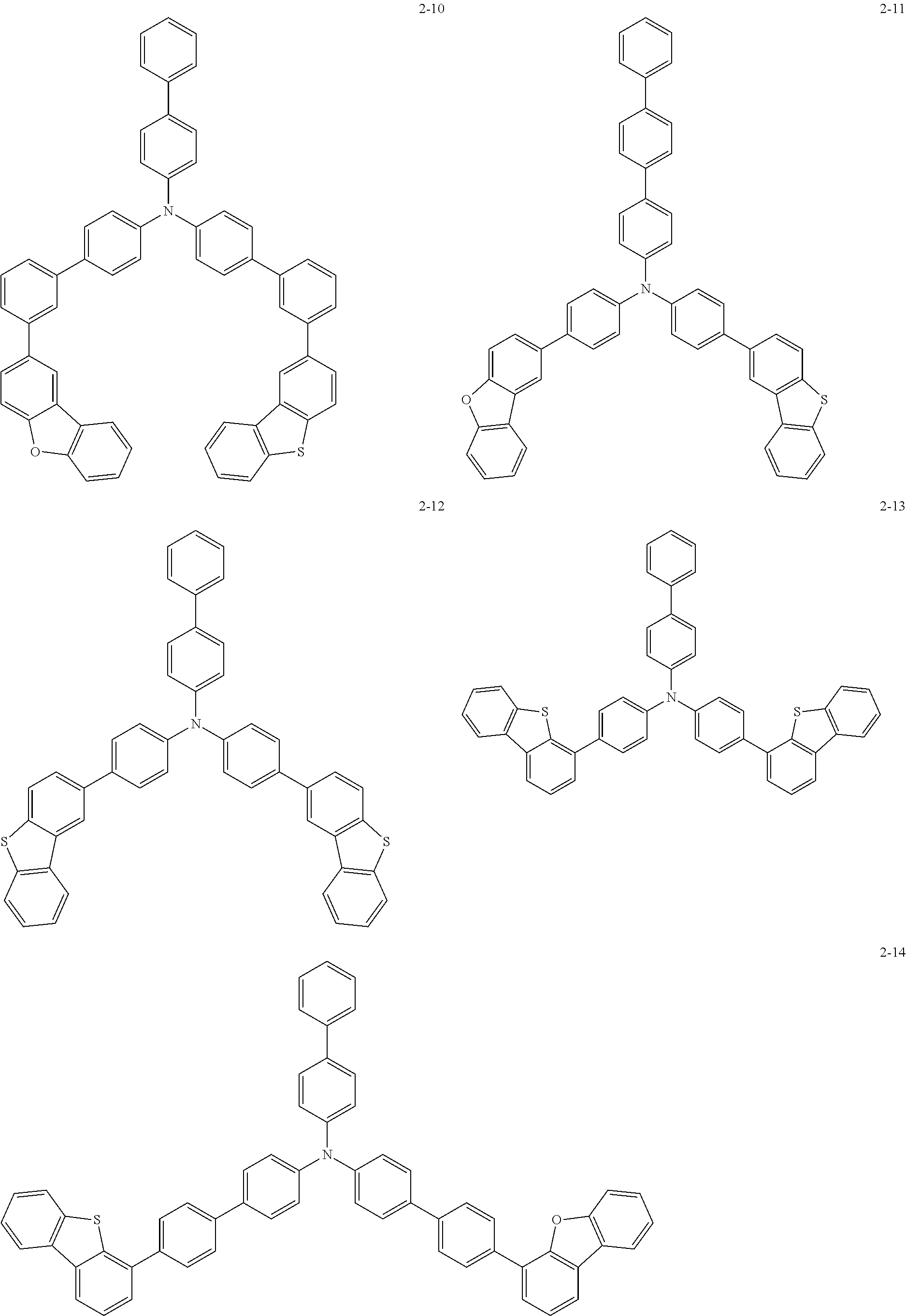

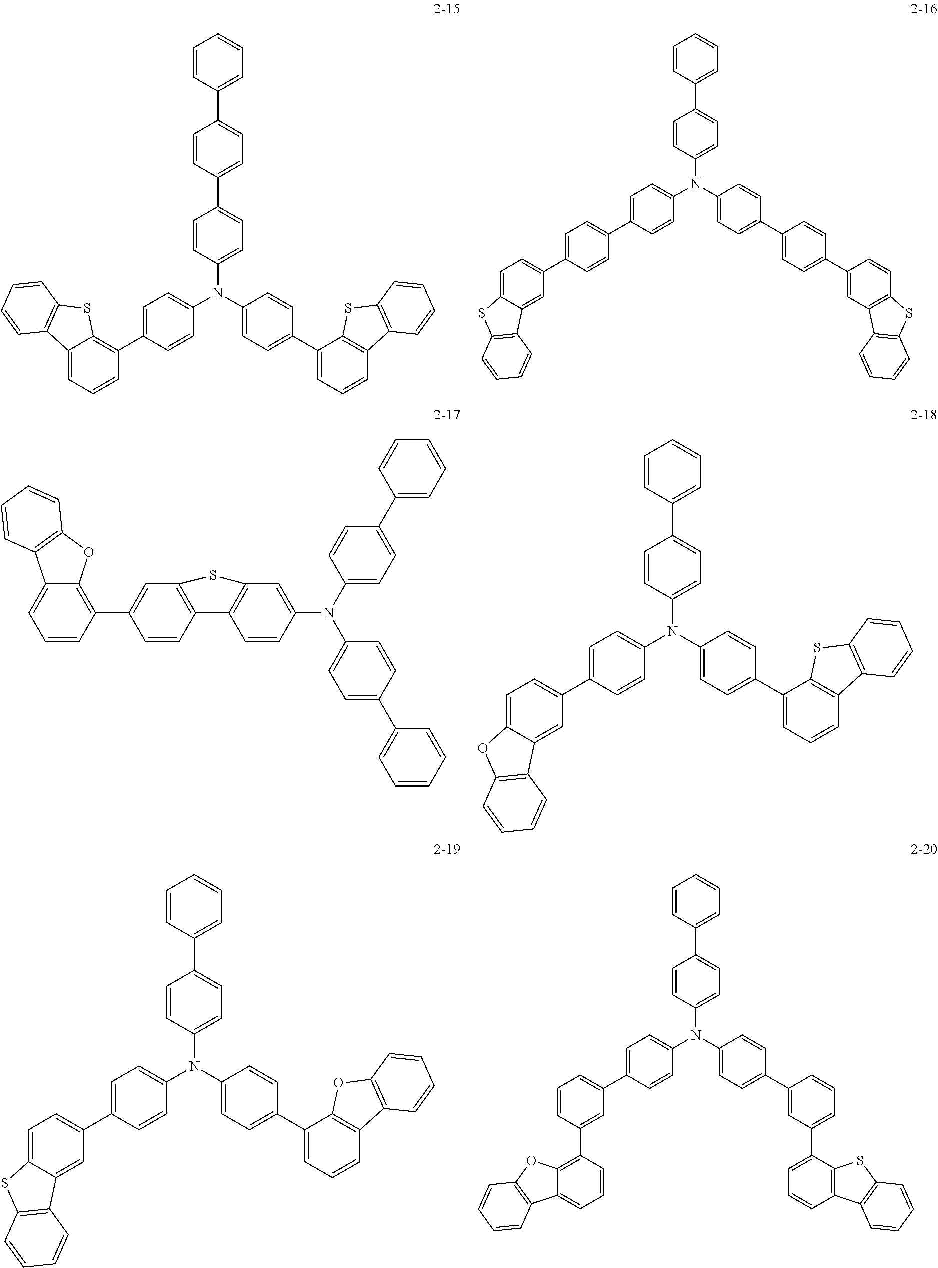

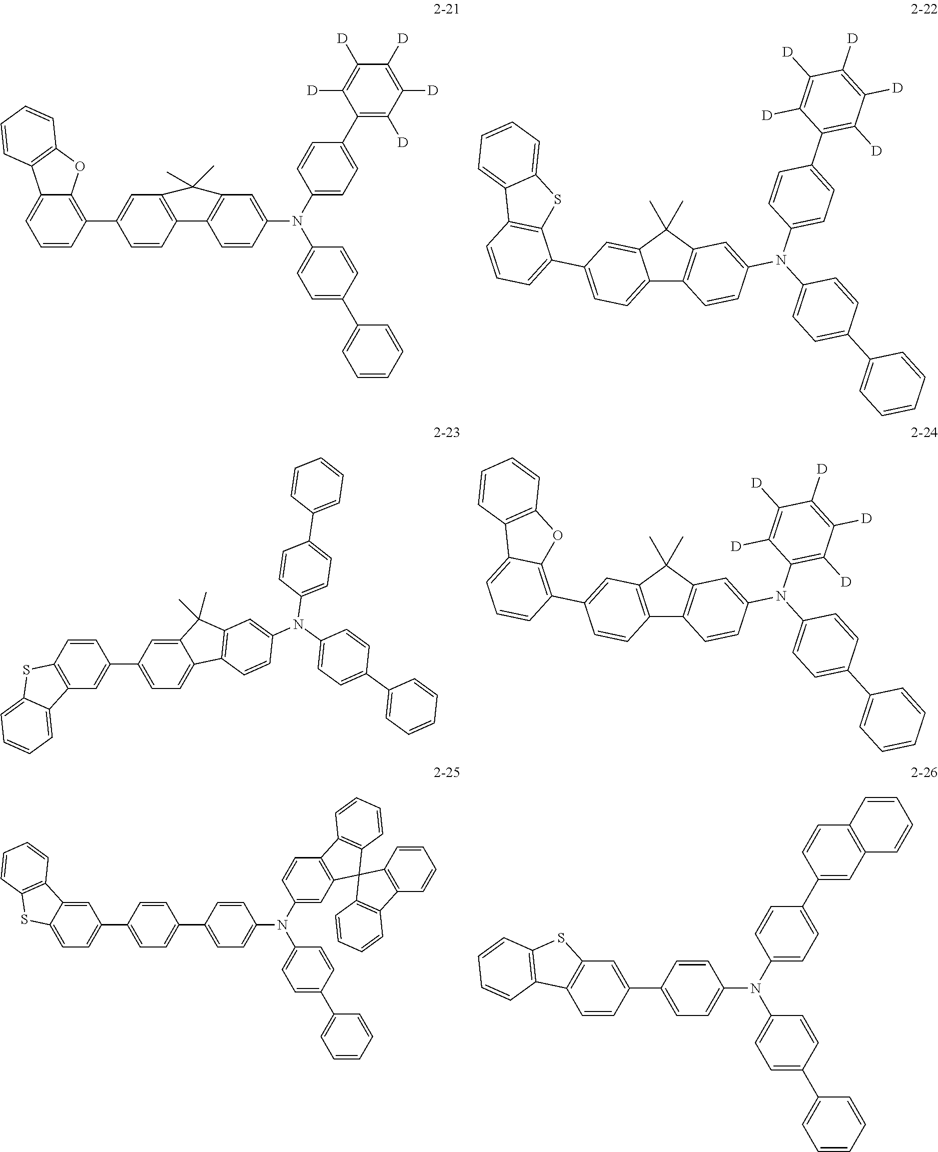

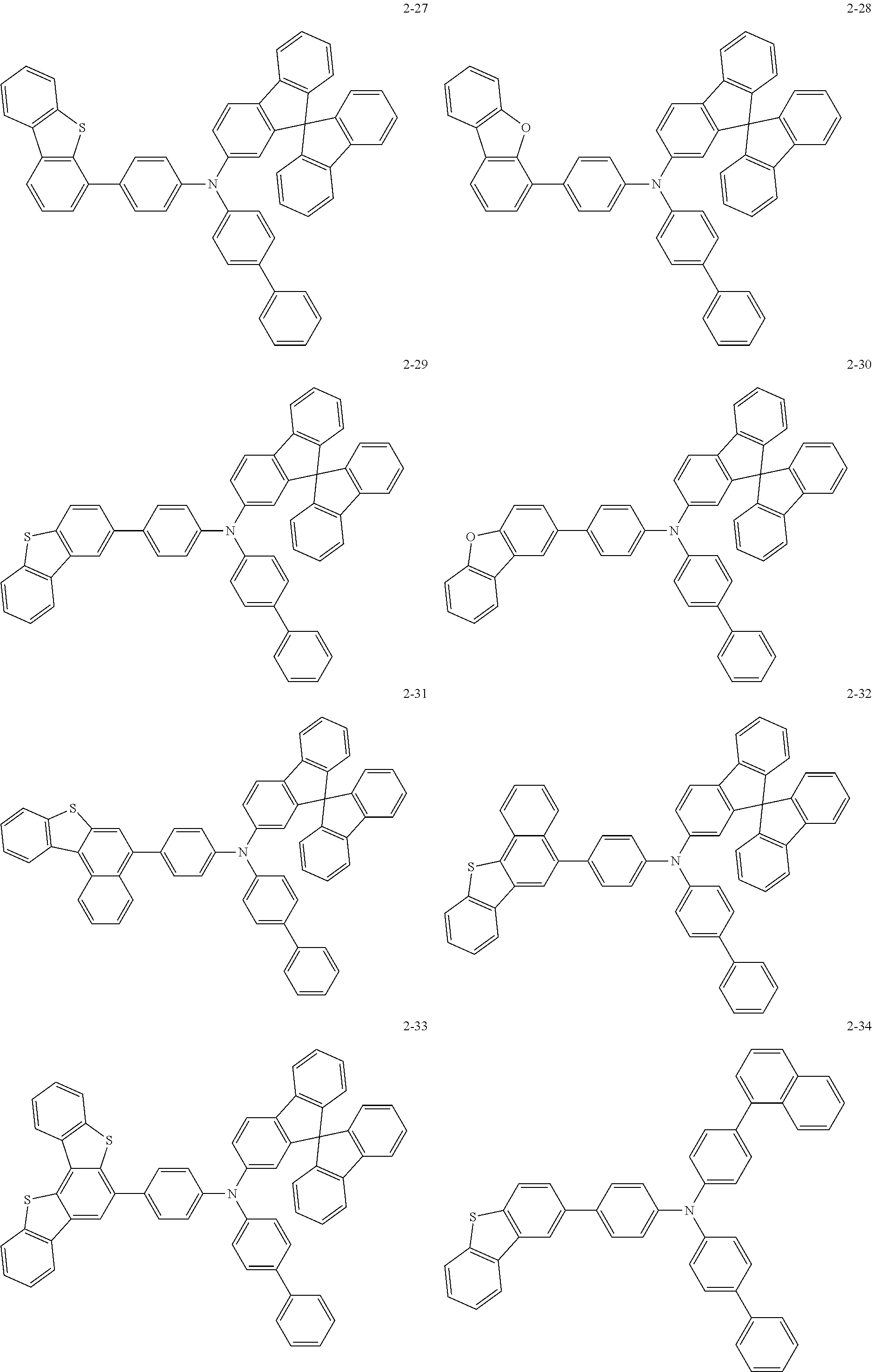

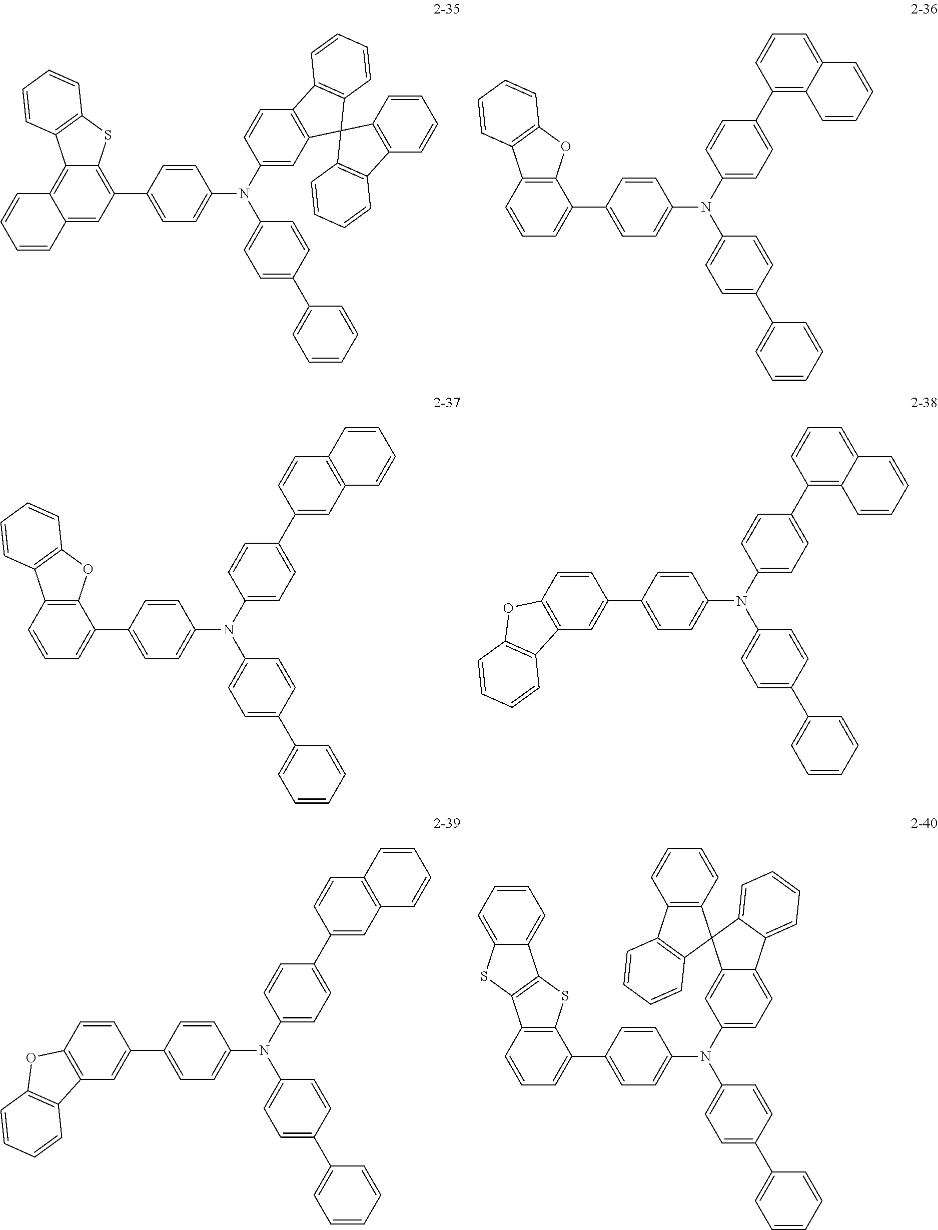

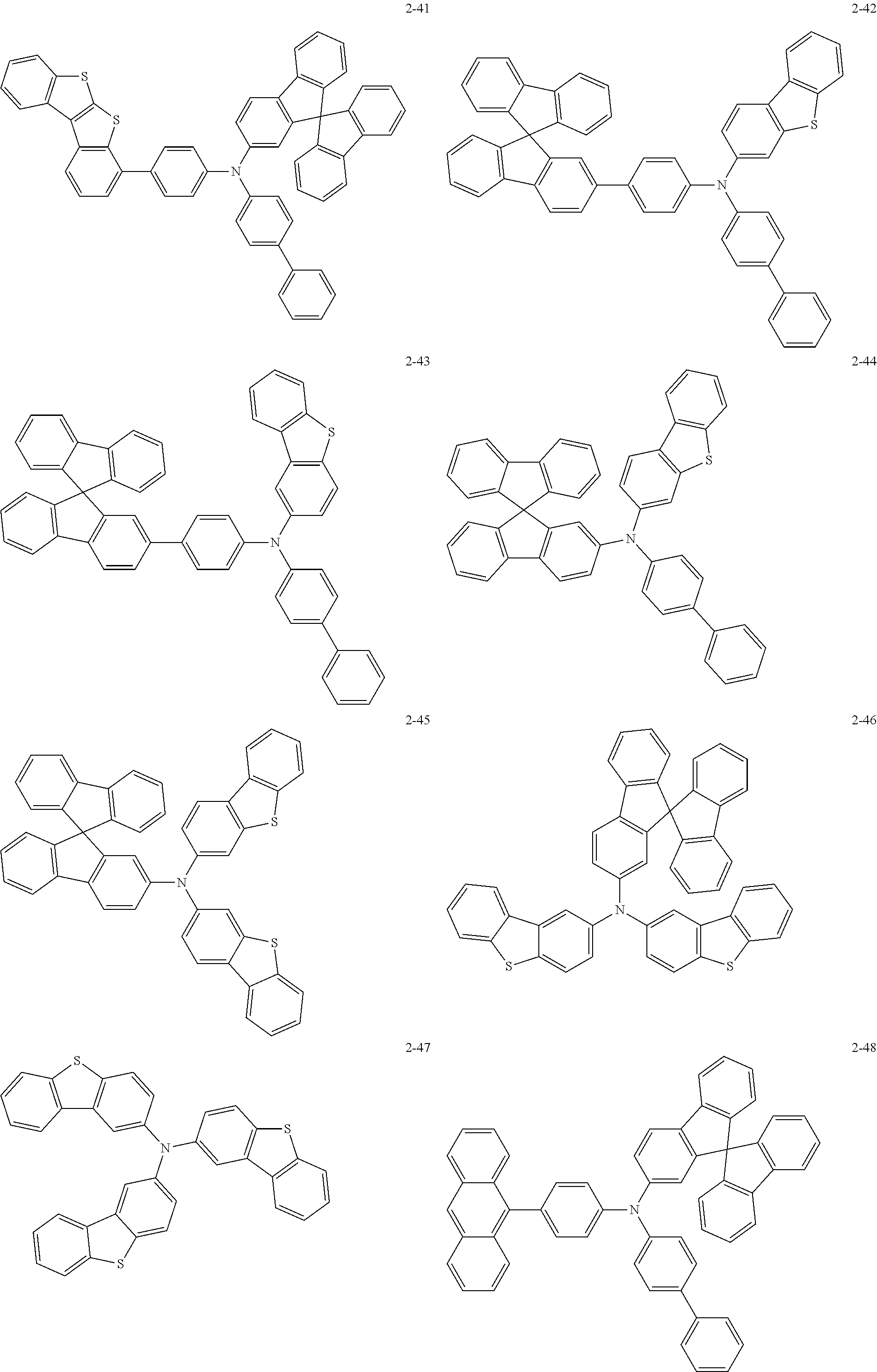

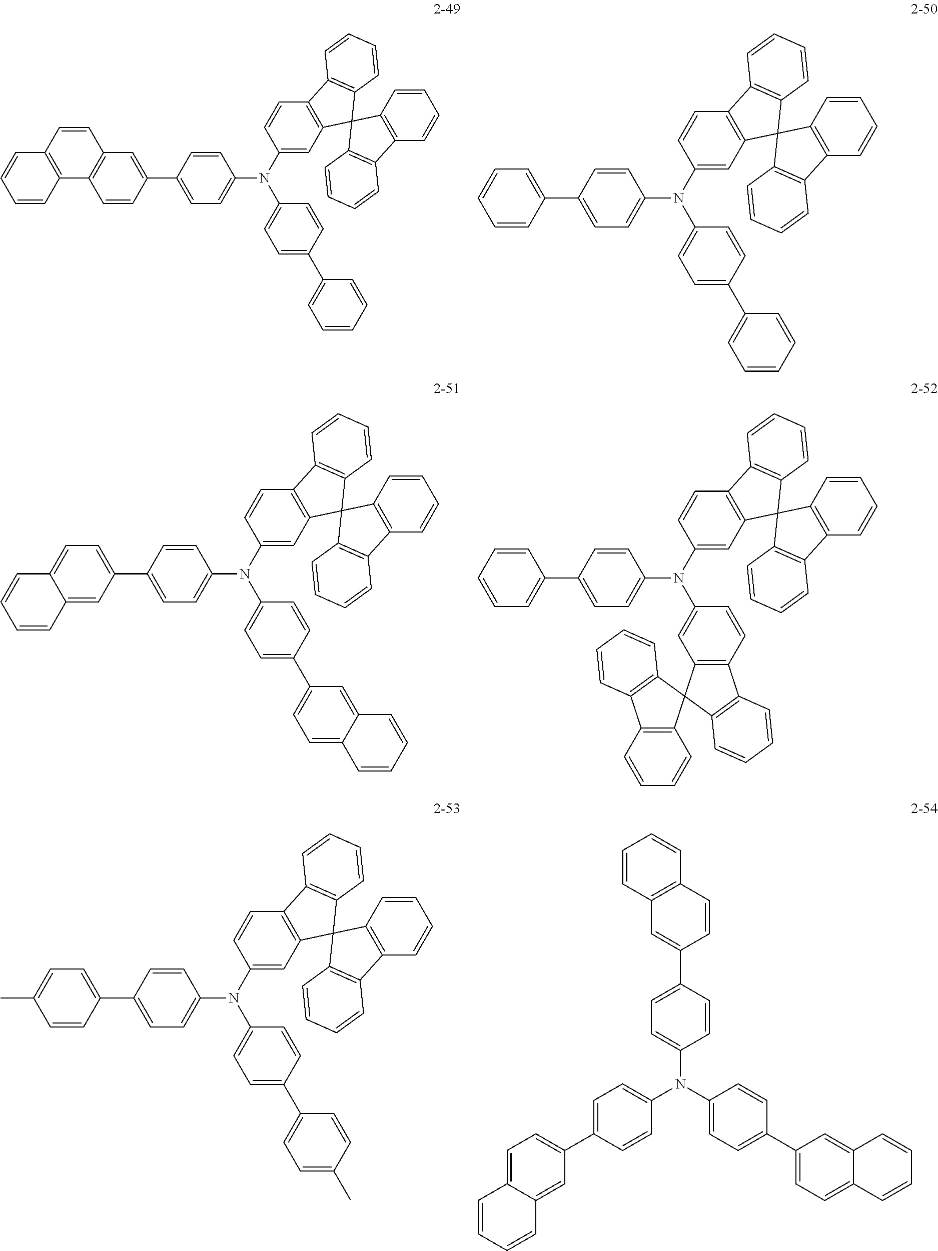

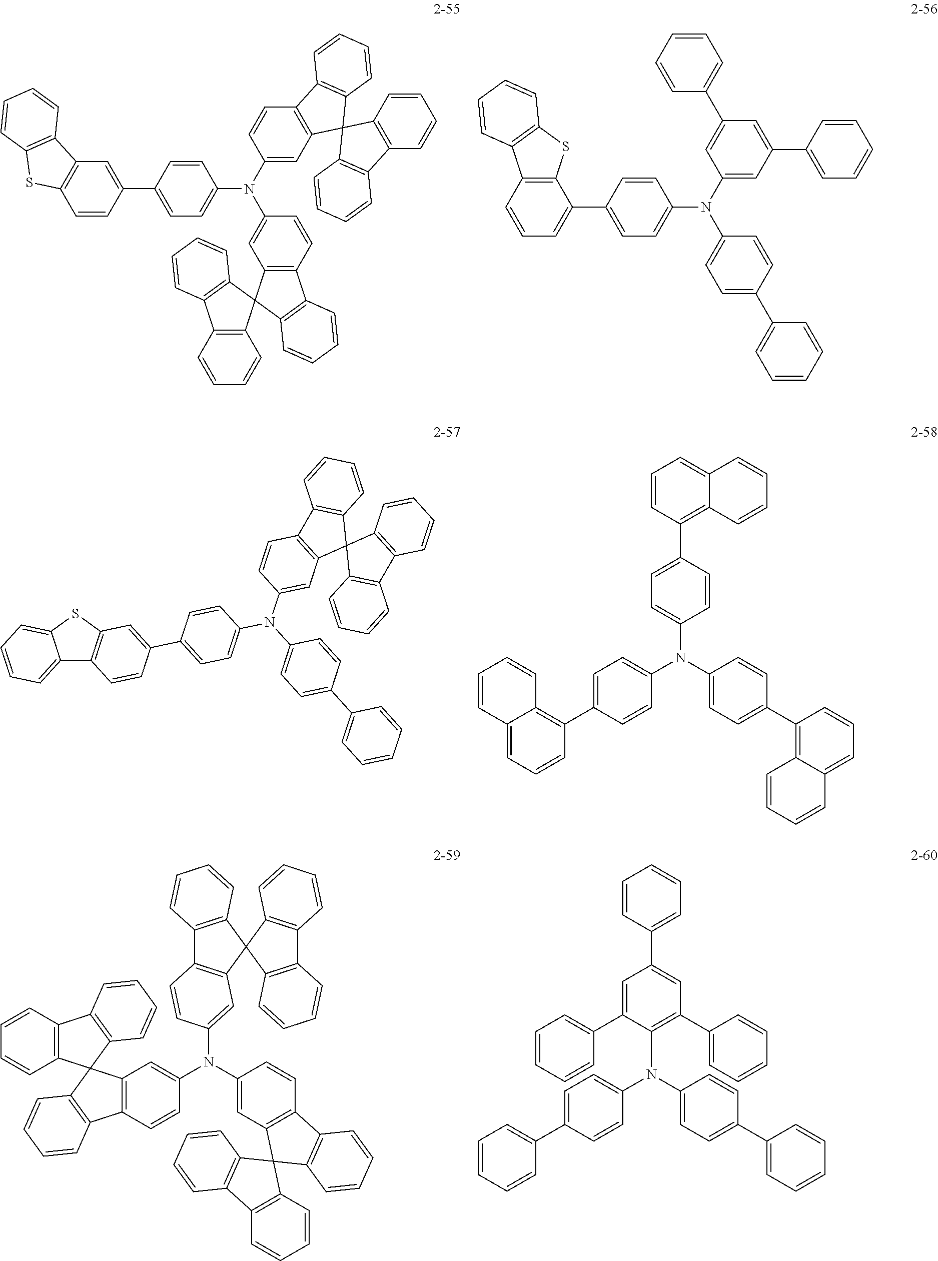

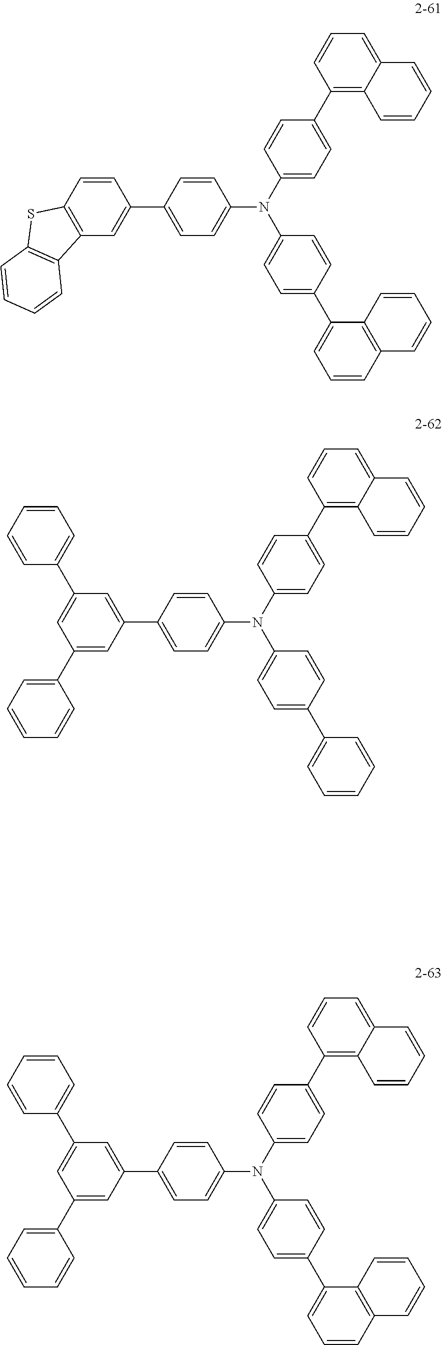 [SMILES: c1ccc2c(-c3ccc(N(c4ccc(-c5ccc6sc7ccccc7c6c5)cc4)c4ccc(-c5cccc6ccccc56)cc4)cc3)cccc2c1 ; c1ccc(-c2ccc(N(c3ccc(-c4cc(-c5ccccc5)cc(-c5ccccc5)c4)cc3)c3ccc(-c4cccc5ccccc45)cc3)cc2)cc1 ; c1ccc(-c2cc(-c3ccccc3)cc(-c3ccc(N(c4ccc(-c5cccc6ccccc56)cc4)c4ccc(-c5cccc6ccccc56)cc4)cc3)c2)cc1]

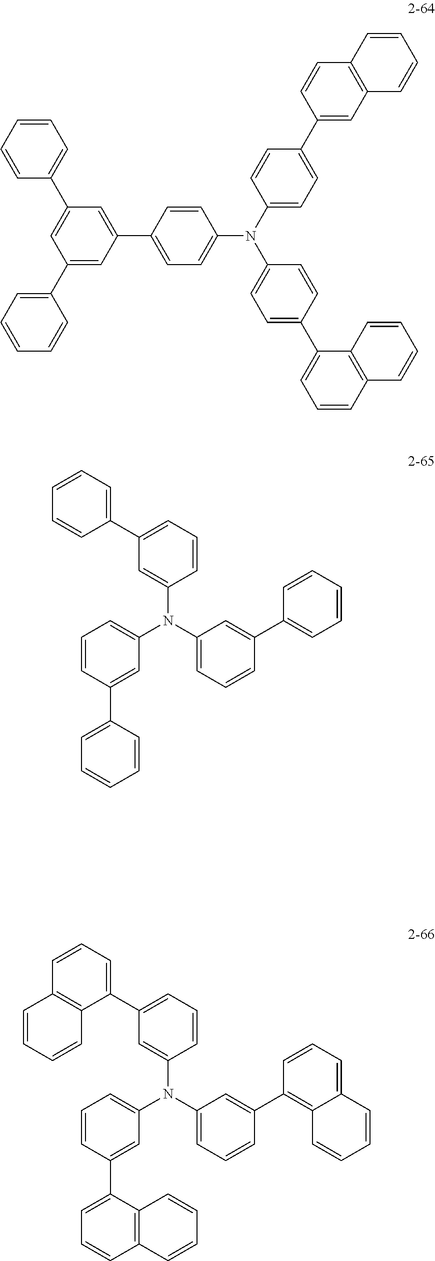 [SMILES: c1ccc(-c2cc(-c3ccccc3)cc(-c3ccc(N(c4ccc(-c5ccc6ccccc6c5)cc4)c4ccc(-c5cccc6ccccc56)cc4)cc3)c2)cc1 ; c1ccc(-c2cccc(N(c3cccc(-c4ccccc4)c3)c3cccc(-c4ccccc4)c3)c2)cc1 ; c1cc(-c2cccc3ccccc23)cc(N(c2cccc(-c3cccc4ccccc34)c2)c2cccc(-c3cccc4ccccc34)c2)c1]

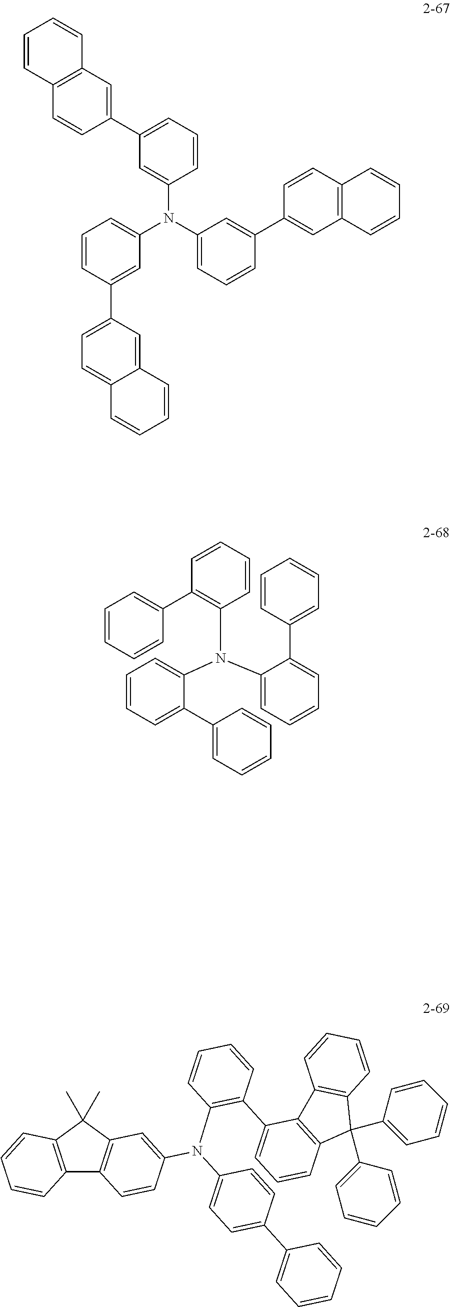 [SMILES: c1cc(-c2ccc3ccccc3c2)cc(N(c2cccc(-c3ccc4ccccc4c3)c2)c2cccc(-c3ccc4ccccc4c3)c2)c1 ; c1ccc(-c2ccccc2N(c2ccccc2-c2ccccc2)c2ccccc2-c2ccccc2)cc1 ; CC1(C)c2ccccc2-c2ccc(N(c3ccc(-c4ccccc4)cc3)c3ccccc3-c3cccc4c3-c3ccccc3C4(c3ccccc3)c3ccccc3)cc21]

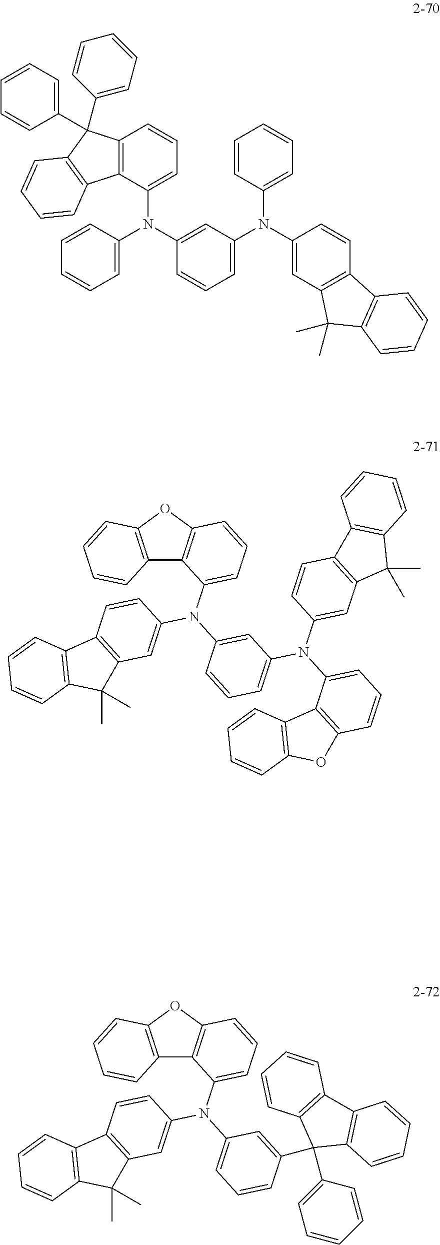 [SMILES: CC1(C)c2ccccc2-c2ccc(N(c3ccccc3)c3cccc(N(c4ccccc4)c4cccc5c4-c4ccccc4C5(c4ccccc4)c4ccccc4)c3)cc21 ; CC1(C)c2ccccc2-c2ccc(N(c3cccc(N(c4ccc5c(c4)C(C)(C)c4ccccc4-5)c4cccc5oc6ccccc6c45)c3)c3cccc4oc5ccccc5c34)cc21 ; CC1(C)c2ccccc2-c2ccc(N(c3cccc(C4(c5ccccc5)c5ccccc5-c5ccccc54)c3)c3cccc4oc5ccccc5c34)cc21]

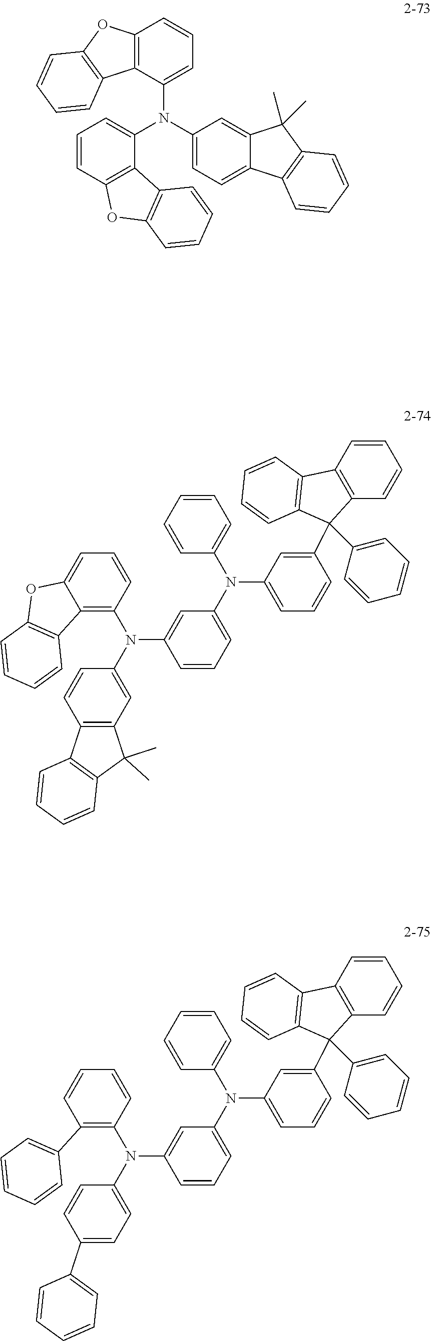 [SMILES: CC1(C)c2ccccc2-c2ccc(N(c3cccc4oc5ccccc5c34)c3cccc4oc5ccccc5c34)cc21 ; CC1(C)c2ccccc2-c2ccc(N(c3cccc(N(c4ccccc4)c4cccc(C5(c6ccccc6)c6ccccc6-c6ccccc65)c4)c3)c3cccc4oc5ccccc5c34)cc21 ; c1ccc(-c2ccc(N(c3cccc(N(c4ccccc4)c4cccc(C5(c6ccccc6)c6ccccc6-c6ccccc65)c4)c3)c3ccccc3-c3ccccc3)cc2)cc1]

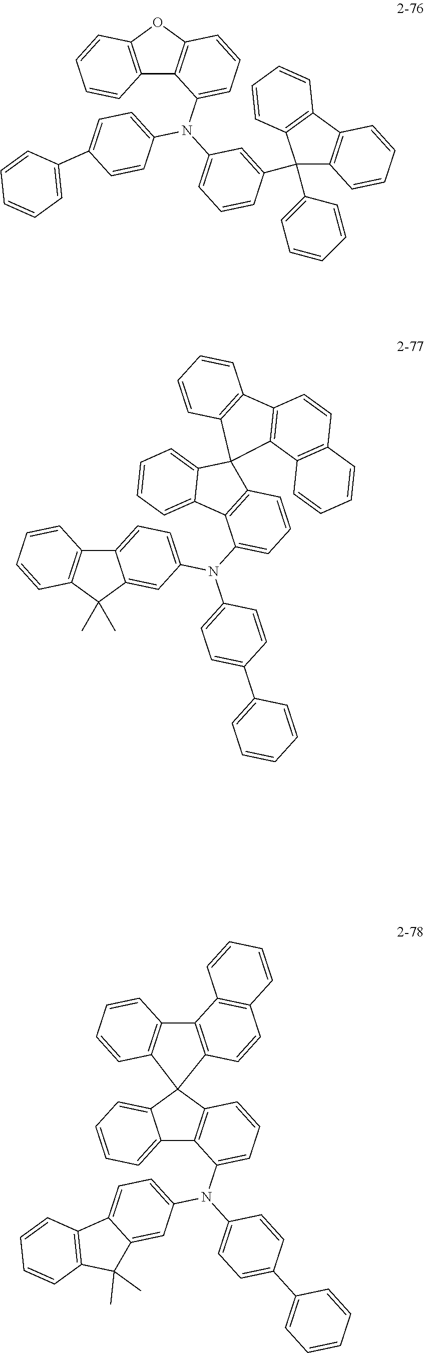 [SMILES: c1ccc(-c2ccc(N(c3cccc(C4(c5ccccc5)c5ccccc5-c5ccccc54)c3)c3cccc4oc5ccccc5c34)cc2)cc1 ; CC1(C)c2ccccc2-c2ccc(N(c3ccc(-c4ccccc4)cc3)c3cccc4c3-c3ccccc3C43c4ccccc4-c4ccc5ccccc5c43)cc21 ; CC1(C)c2ccccc2-c2ccc(N(c3ccc(-c4ccccc4)cc3)c3cccc4c3-c3ccccc3C43c4ccccc4-c4c3ccc3ccccc43)cc21]

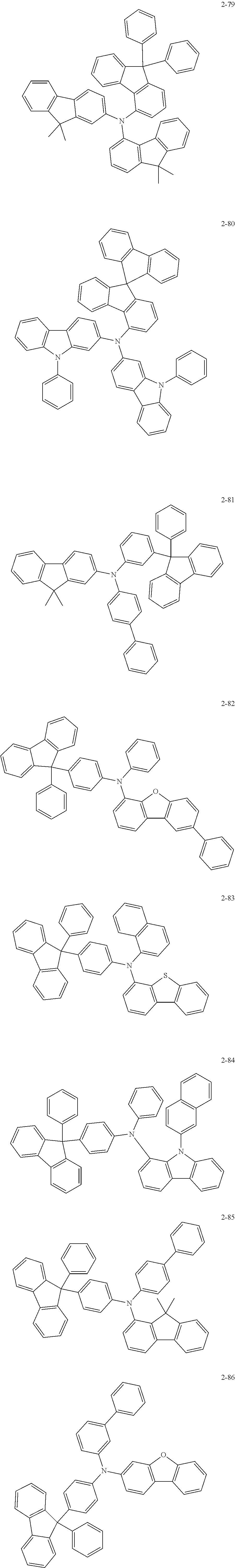

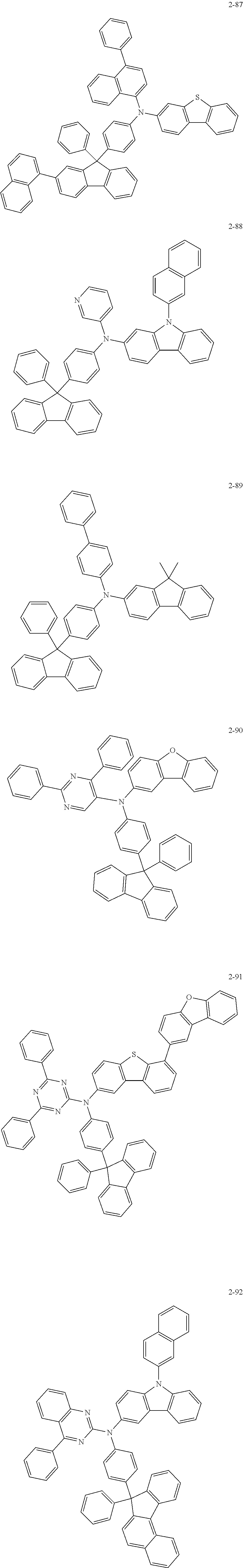

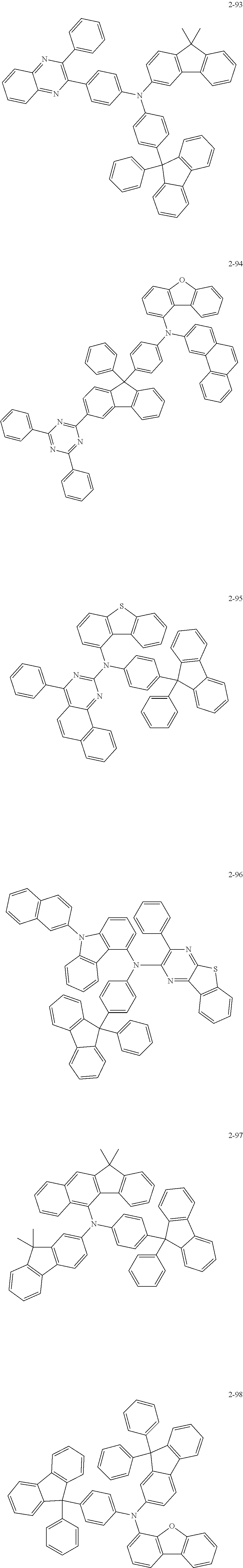

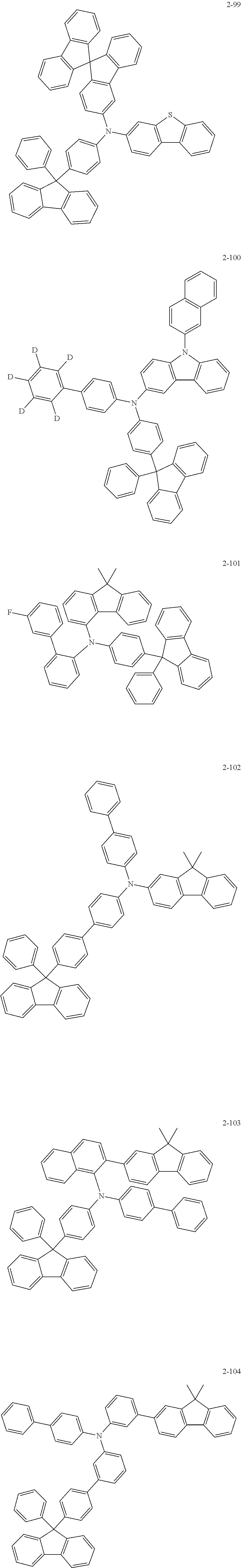

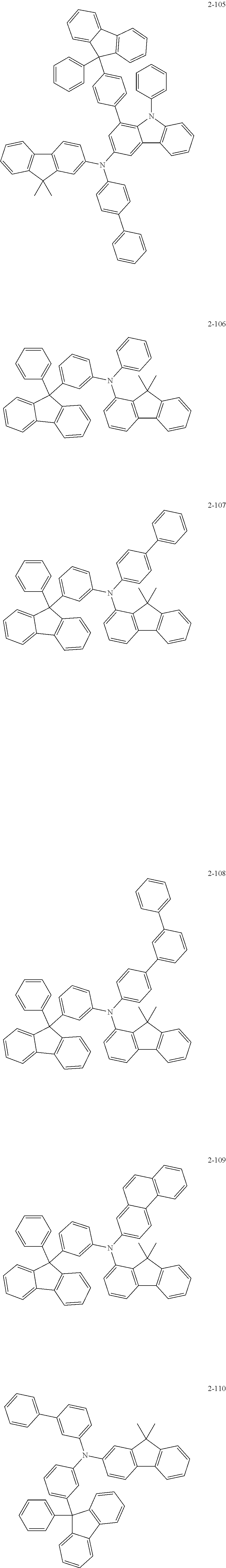

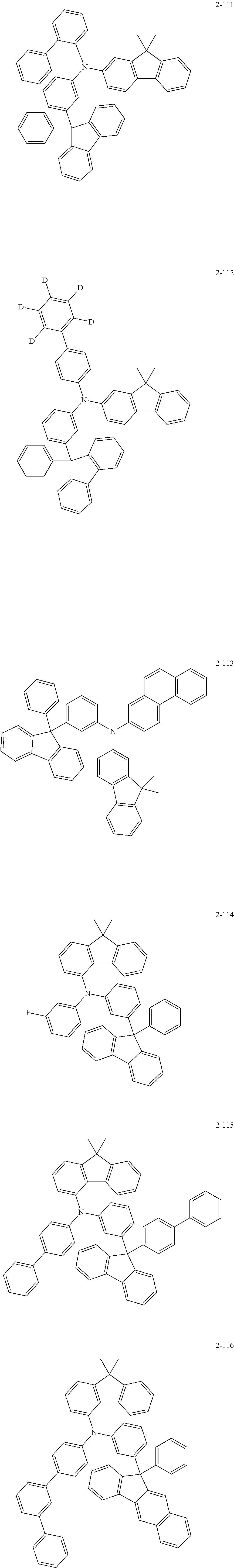

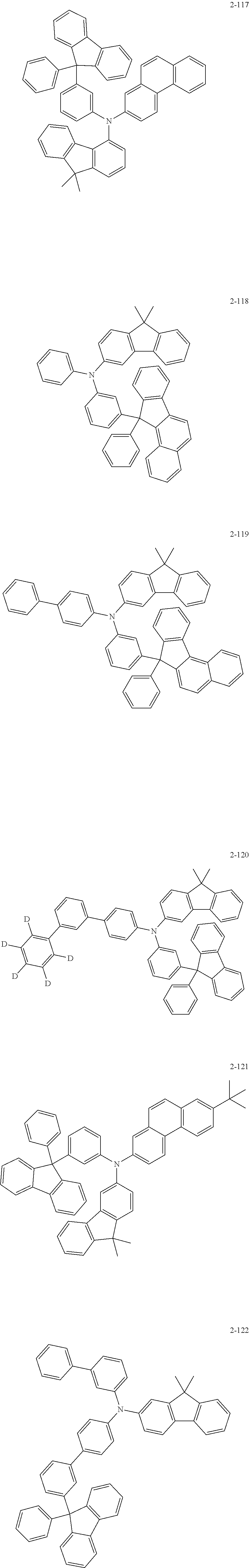

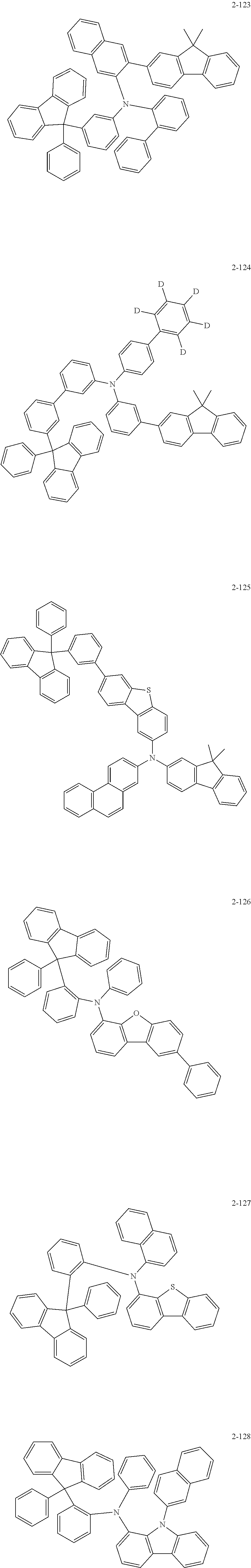

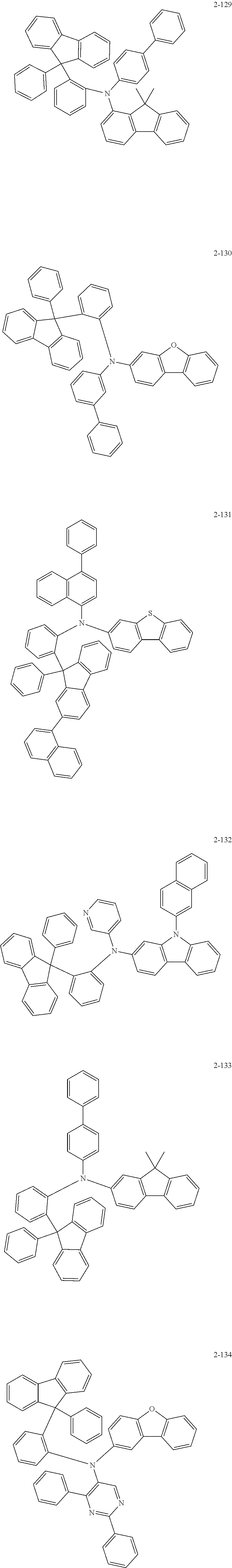

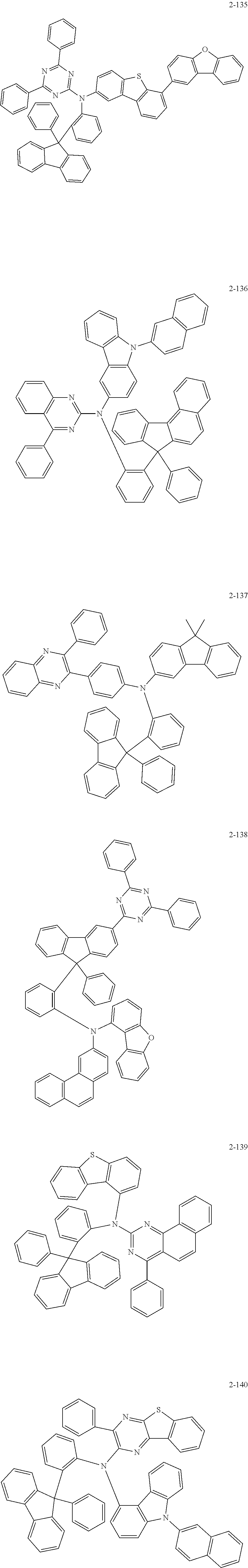

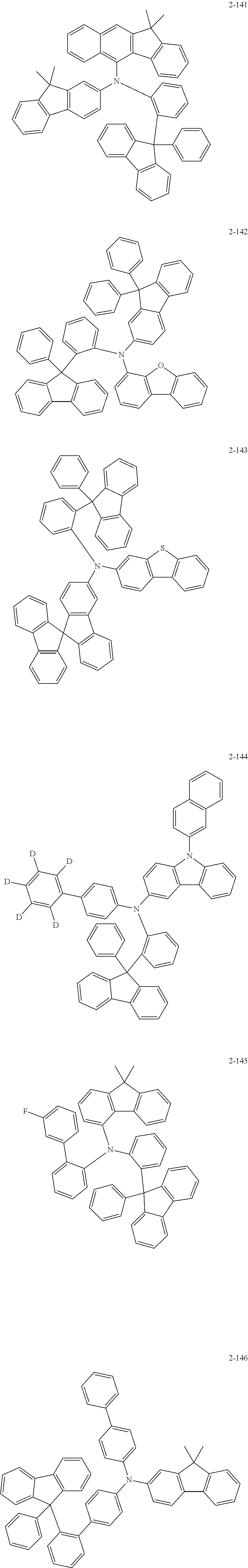

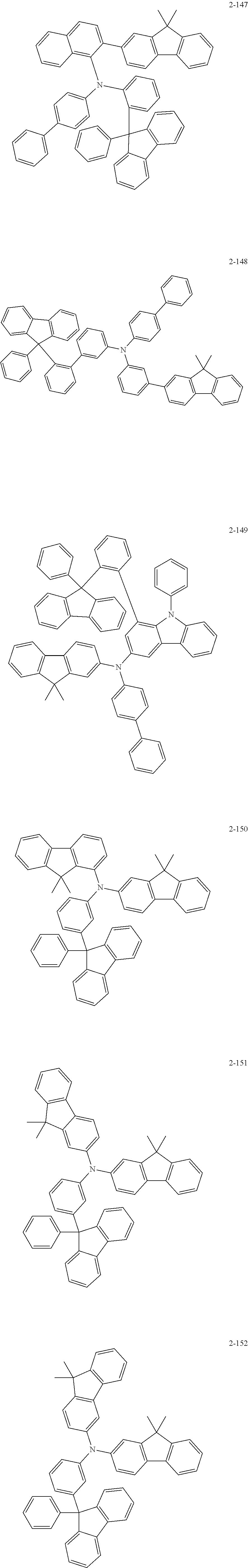

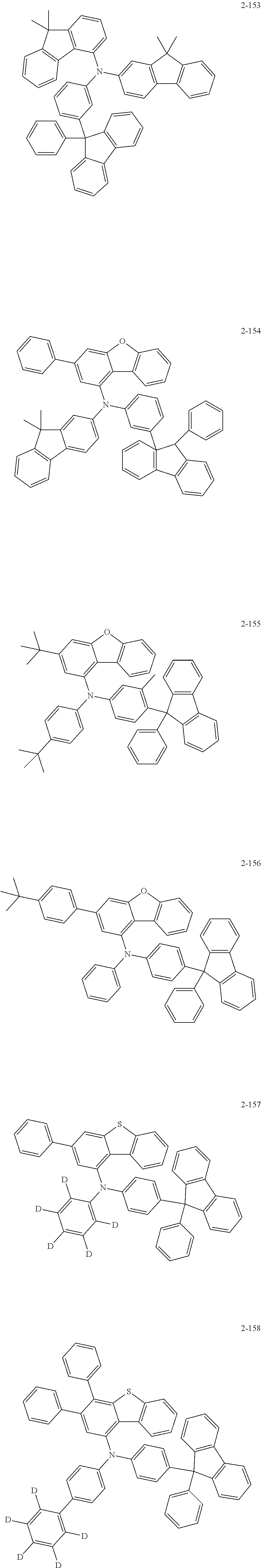

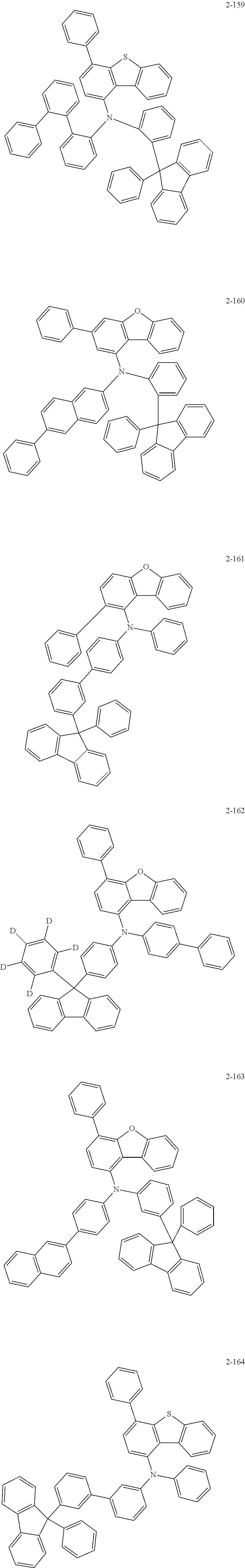

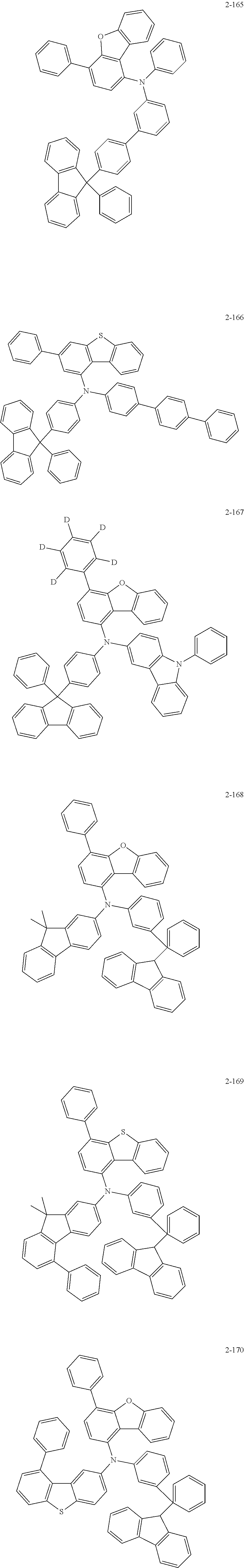

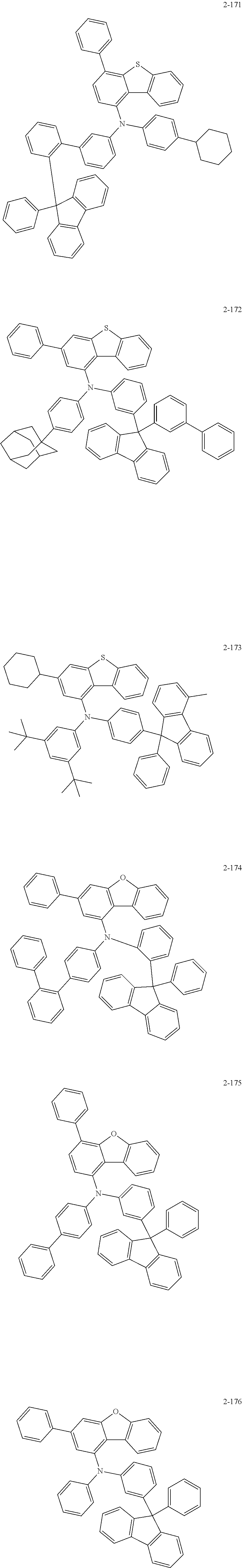

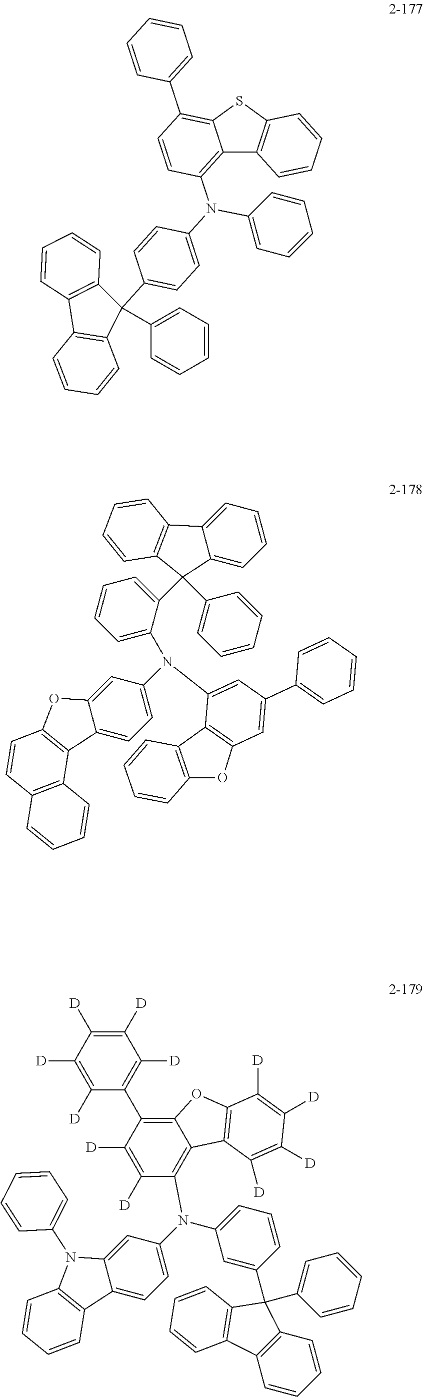 [SMILES: c1ccc(-c2ccc(N(c3ccccc3)c3ccc(C4(c5ccccc5)c5ccccc5-c5ccccc54)cc3)c3c2sc2ccccc23)cc1 ; c1ccc(-c2cc(N(c3ccc4c(c3)oc3ccc5ccccc5c34)c3ccccc3C3(c4ccccc4)c4ccccc4-c4ccccc43)c3c(c2)oc2ccccc23)cc1 ; [2H]c1c([2H])c([2H])c(-c2c([2H])c([2H])c(N(c3cccc(C4(c5ccccc5)c5ccccc5-c5ccccc54)c3)c3ccc4c5ccccc5n(-c5ccccc5)c4c3)c3c2oc2c([2H])c([2H])c([2H])c([2H])c23)c([2H])c1[2H]]

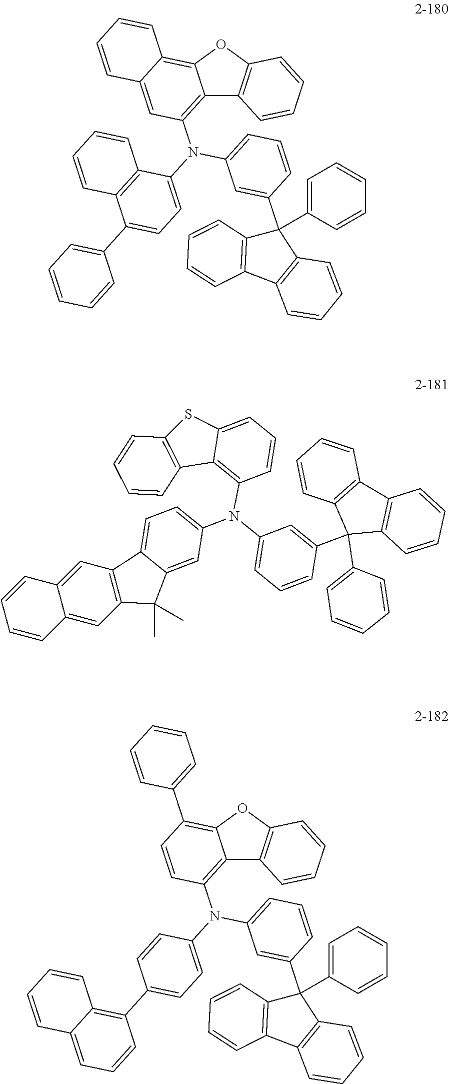 [SMILES: c1ccc(-c2ccc(N(c3cccc(C4(c5ccccc5)c5ccccc5-c5ccccc54)c3)c3cc4ccccc4c4oc5ccccc5c34)c3ccccc23)cc1 ; CC1(C)c2cc(N(c3cccc(C4(c5ccccc5)c5ccccc5-c5ccccc54)c3)c3cccc4sc5ccccc5c34)ccc2-c2cc3ccccc3cc21 ; c1ccc(-c2ccc(N(c3ccc(-c4cccc5ccccc45)cc3)c3cccc(C4(c5ccccc5)c5ccccc5-c5ccccc54)c3)c3c2oc2ccccc23)cc1]

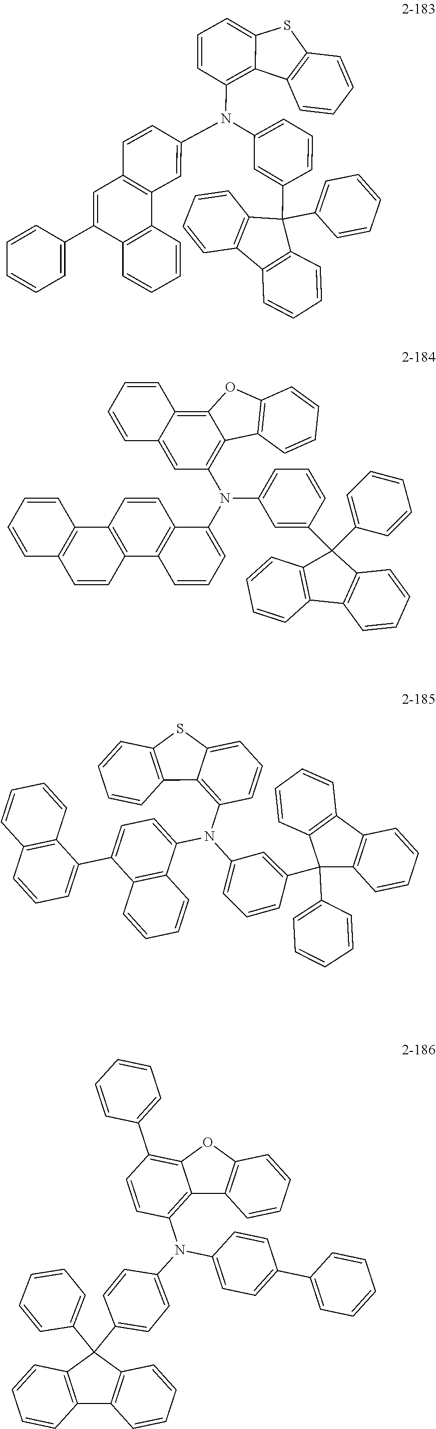 [SMILES: c1ccc(-c2cc3ccc(N(c4cccc(C5(c6ccccc6)c6ccccc6-c6ccccc65)c4)c4cccc5sc6ccccc6c45)cc3c3ccccc23)cc1 ; c1ccc(C2(c3cccc(N(c4cccc5c4ccc4c6ccccc6ccc54)c4cc5ccccc5c5oc6ccccc6c45)c3)c3ccccc3-c3ccccc32)cc1 ; c1ccc(C2(c3cccc(N(c4ccc(-c5cccc6ccccc56)c5ccccc45)c4cccc5sc6ccccc6c45)c3)c3ccccc3-c3ccccc32)cc1 ; c1ccc(-c2ccc(N(c3ccc(C4(c5ccccc5)c5ccccc5-c5ccccc54)cc3)c3ccc(-c4ccccc4)c4oc5ccccc5c34)cc2)cc1]

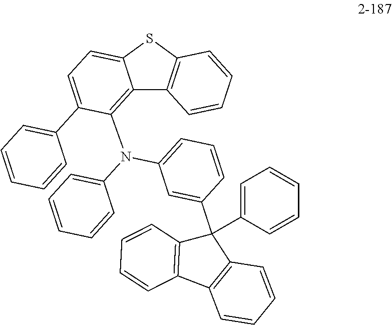 [SMILES: c1ccc(-c2ccc3sc4ccccc4c3c2N(c2ccccc2)c2cccc(C3(c4ccccc4)c4ccccc4-c4ccccc43)c2)cc1]

As another example, the present invention provides an organic electronic element comprising an anode; a cathode; an organic material layer formed between the anode and the cathode; wherein the organic material layer includes an emitting layer, an hole transport layer formed between the anode and the emitting layer; an emitting auxiliary layer or an electron blocking layer (EBL) formed between the emitting layer and the hole transport layer; wherein the emitting auxiliary layer or the electron blocking layer comprises a compound represented by Formula (30).

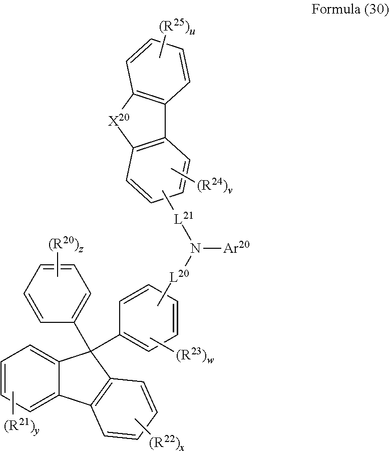

{In Formula (30),

R²⁰, R²¹, R²², R²³, R²⁴, and R²⁵ are each independently selected from the group consisting of hydrogen; deuterium; halogen; a C₆-C₃₀ aryl group; a fluorenyl group; a C₂-C₃₀ heterocyclic group including at least one heteroatom of O, N, S, Si or P; a fused ring group of a C3-Cso aliphatic ring and a C6-Cso aromatic ring; a C₁-C₃₀ alkyl group; a C₂-C₂₀ alkenyl group; a C₂-C₂₀ alkynyl group; a C₁-C₃₀ alkoxyl group; a C₆-C₃₀ aryloxy group; or plurality of R²⁰, plurality of R²², plurality of R²³, plurality of R²⁴, and plurality of R²⁵ may be bonded to each other to form an aromatic ring or and heteroaromatic ring,

v is an integer of 0 to 3,

u, w, x and y are each independently an integer of 0 to 4,

Z is an integer of 0 to 5,

L²⁰ and L²¹ are each independently a single bond; a C₆-C₃₀ arylene group; a C₃-C₃₀ heteroarylene group;

Ar²⁰ is a C₆-C₃₀ aryl group; or a C₃-C₃₀ heteroarylene group;

X²⁰ is O, S, NR′ or CR′R″

R′ and R″are each independently selected from the group of a C₁-C₃₀ alkyl group; a C₆-C₃₀ aryl group; a C₃-C₃₀ heterocyclic group including at least one heteroatom of O, N, S, Si, or P; and R′ and R″ may be bonded to each other to form a spiro.

wherein, the aryl group, fluorenyl group, arylene group, heterocyclic group, fluorenylene group, fused ring group, alkyl group, alkenyl group, alkoxy group and aryloxy group may be substituted with one or more substituents selected from the group consisting of deuterium; halogen; silane group; siloxane group; boron group; germanium group; cyano group; nitro group; C₁-C₂₀ alkylthio group; C₁-C₂₀ alkoxyl group; C₁-C₂₀ alkyl group; C₂-C₂₀ alkenyl group; C₂-C₂₀ alkynyl group; C₆-C₂₀ aryl group; C₆-C₂₀ aryl group substituted with deuterium; a fluorenyl group; C₂-C₂₀ heterocyclic group; C₃-C₂₀ cycloalkyl group; C₇-C₂₀ arylalkyl group and C₈-C₂₀ arylalkenyl group, wherein the substituents may be bonded to each other to form a ring, wherein the term ‘ring’ means a C₃-C₆₀ aliphatic ring or a C₆-C₆₀ aromatic ring or a C₂-C₆₀ heterocyclic group or a fused ring formed by the combination thereof and comprises a saturated or unsaturated ring.}

In present invention, the compound represented by Formula (30) is represented by any of the Formulas (31) to (40)

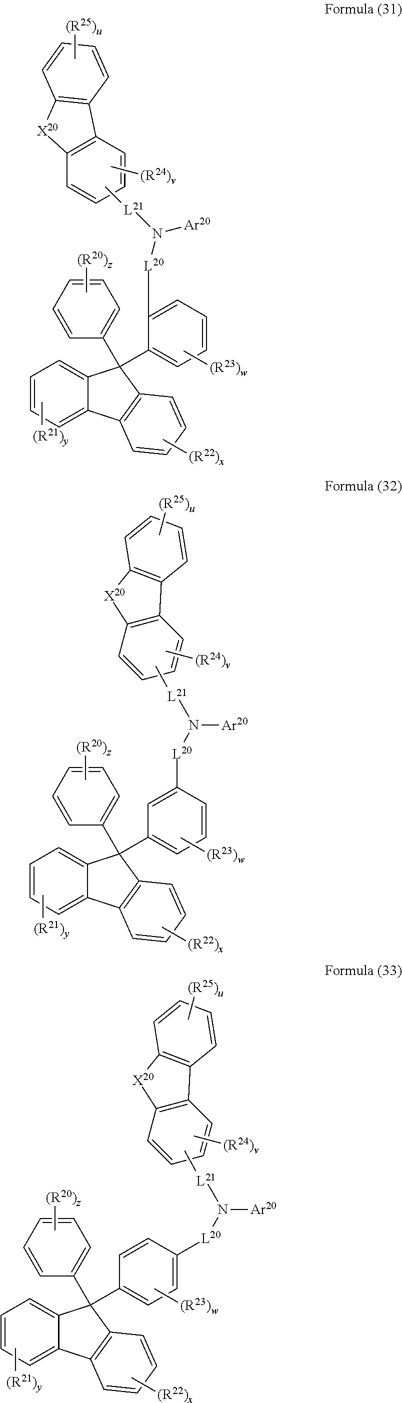

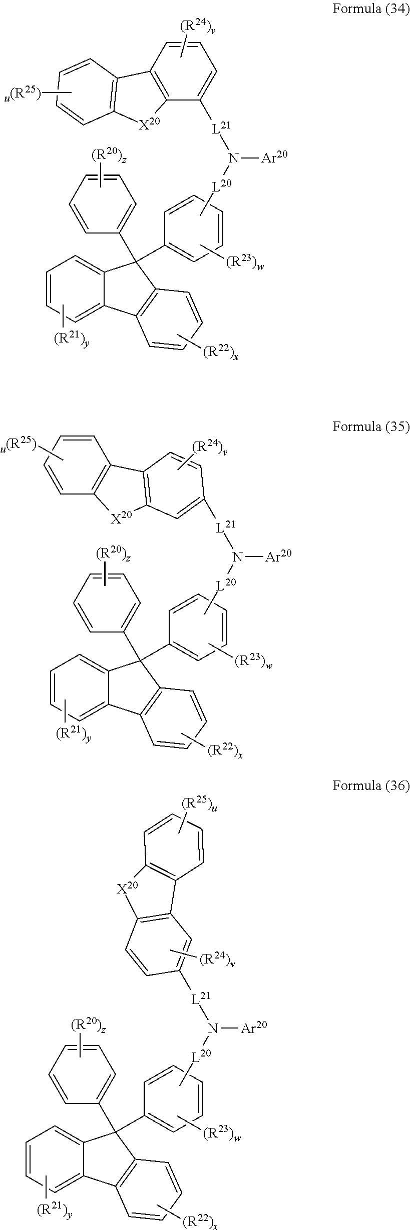

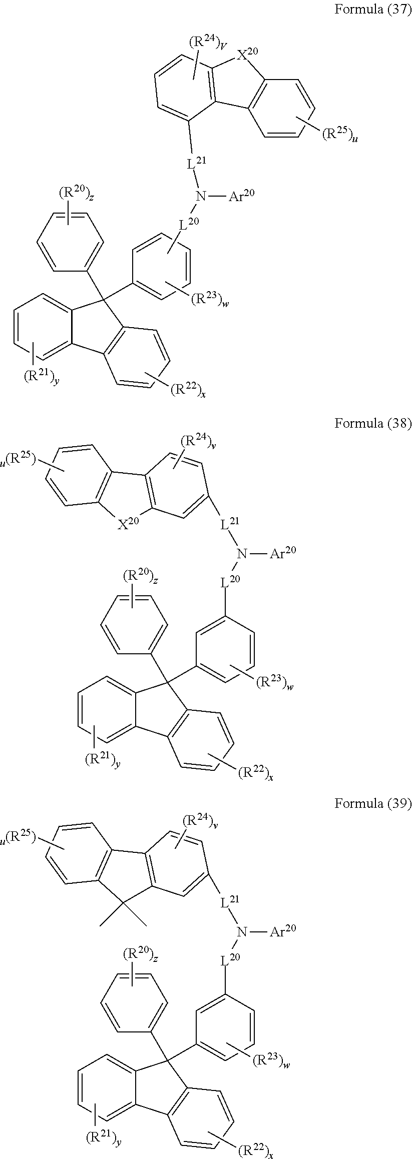

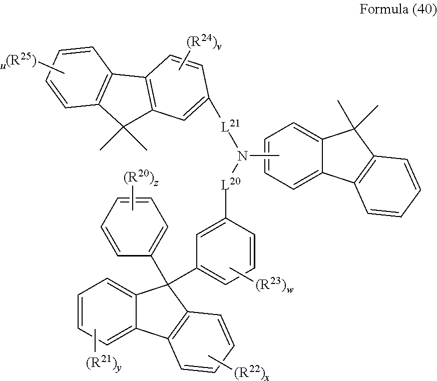

{In Formulas (31) to (40),

R²⁰, R²¹, R²², R²³, R²⁴, R²⁵, L²⁰, L²¹, Ar²⁰ and X²⁰, u, v, w, x, y and z are the same as defined above.}

In the present invention, the compound represented by Formula (30) comprises the following compounds.

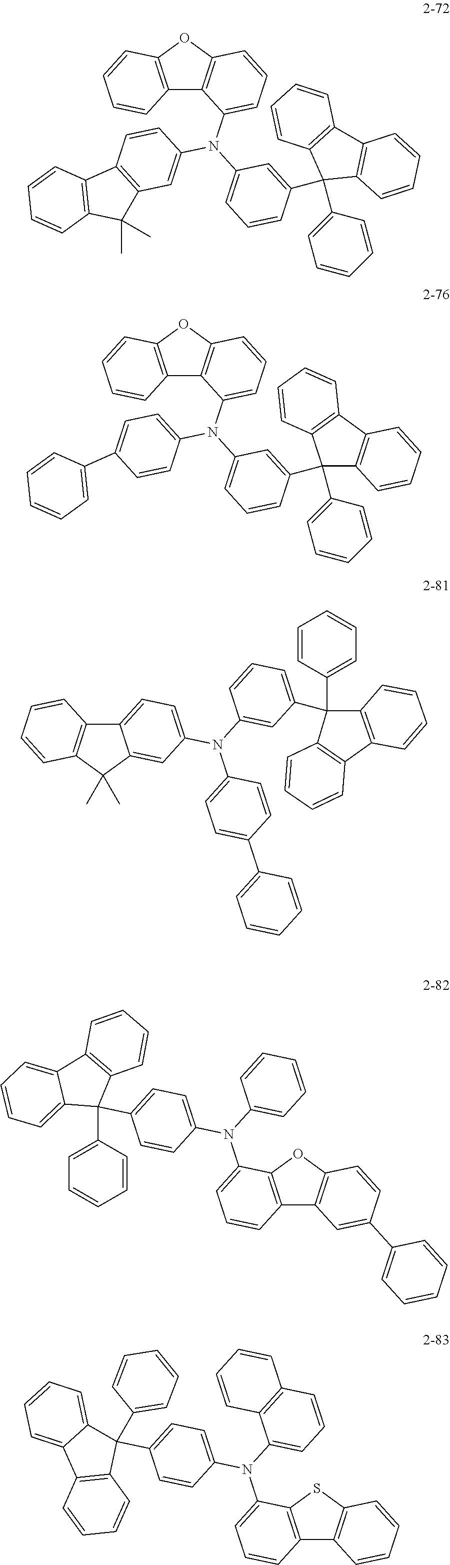 [SMILES: CC1(C)c2ccccc2-c2ccc(N(c3cccc(C4(c5ccccc5)c5ccccc5-c5ccccc54)c3)c3cccc4oc5ccccc5c34)cc21 ; c1ccc(-c2ccc(N(c3cccc(C4(c5ccccc5)c5ccccc5-c5ccccc54)c3)c3cccc4oc5ccccc5c34)cc2)cc1 ; CC1(C)c2ccccc2-c2ccc(N(c3ccc(-c4ccccc4)cc3)c3cccc(C4(c5ccccc5)c5ccccc5-c5ccccc54)c3)cc21 ; c1ccc(-c2ccc3oc4c(N(c5ccccc5)c5ccc(C6(c7ccccc7)c7ccccc7-c7ccccc76)cc5)cccc4c3c2)cc1 ; c1ccc(C2(c3ccc(N(c4cccc5ccccc45)c4cccc5c4sc4ccccc45)cc3)c3ccccc3-c3ccccc32)cc1]

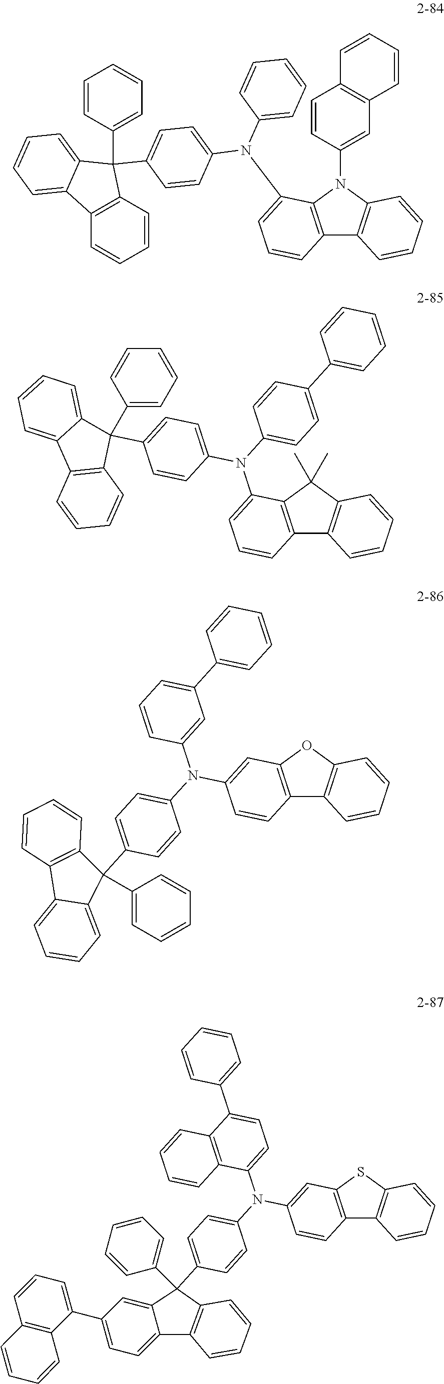 [SMILES: c1ccc(N(c2ccc(C3(c4ccccc4)c4ccccc4-c4ccccc43)cc2)c2cccc3c4ccccc4n(-c4ccc5ccccc5c4)c23)cc1 ; CC1(C)c2ccccc2-c2cccc(N(c3ccc(-c4ccccc4)cc3)c3ccc(C4(c5ccccc5)c5ccccc5-c5ccccc54)cc3)c21 ; c1ccc(-c2cccc(N(c3ccc(C4(c5ccccc5)c5ccccc5-c5ccccc54)cc3)c3ccc4c(c3)oc3ccccc34)c2)cc1 ; c1ccc(-c2ccc(N(c3ccc(C4(c5ccccc5)c5ccccc5-c5ccc(-c6cccc7ccccc67)cc54)cc3)c3ccc4c(c3)sc3ccccc34)c3ccccc23)cc1]

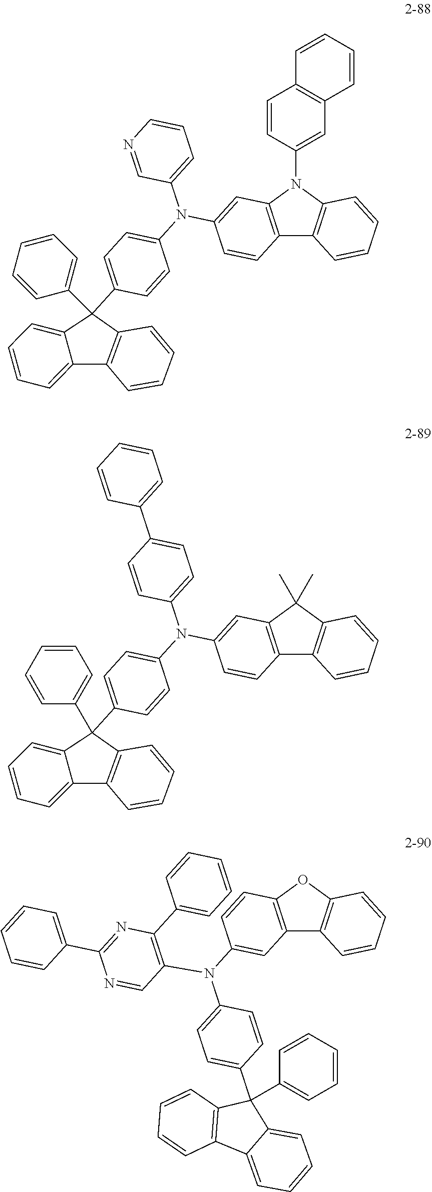 [SMILES: c1ccc(C2(c3ccc(N(c4cccnc4)c4ccc5c6ccccc6n(-c6ccc7ccccc7c6)c5c4)cc3)c3ccccc3-c3ccccc32)cc1 ; CC1(C)c2ccccc2-c2ccc(N(c3ccc(-c4ccccc4)cc3)c3ccc(C4(c5ccccc5)c5ccccc5-c5ccccc54)cc3)cc21 ; c1ccc(-c2ncc(N(c3ccc(C4(c5ccccc5)c5ccccc5-c5ccccc54)cc3)c3ccc4oc5ccccc5c4c3)c(-c3ccccc3)n2)cc1]

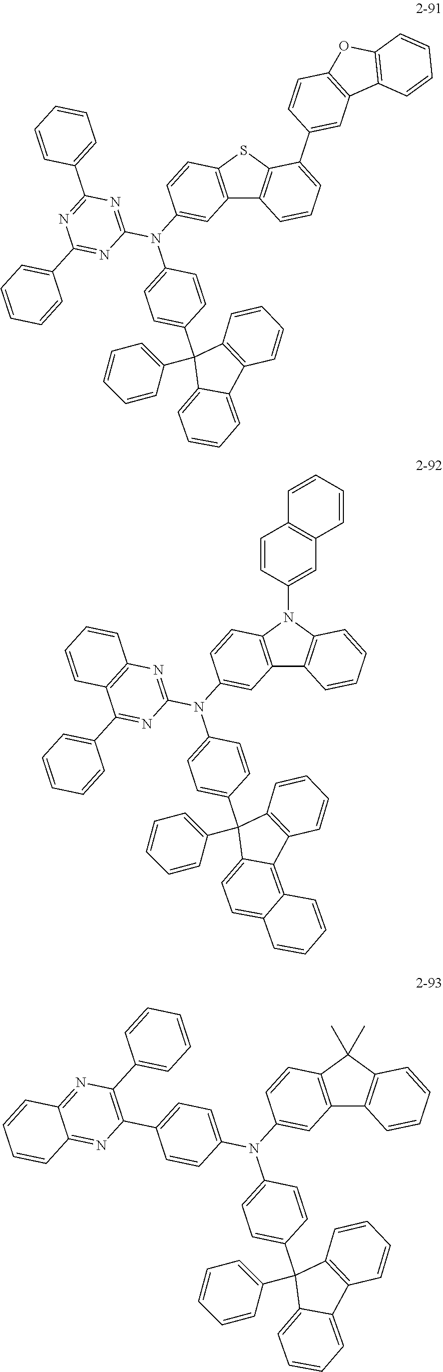 [SMILES: c1ccc(-c2nc(-c3ccccc3)nc(N(c3ccc(C4(c5ccccc5)c5ccccc5-c5ccccc54)cc3)c3ccc4sc5c(-c6ccc7oc8ccccc8c7c6)cccc5c4c3)n2)cc1 ; c1ccc(-c2nc(N(c3ccc(C4(c5ccccc5)c5ccccc5-c5c4ccc4ccccc54)cc3)c3ccc4c(c3)c3ccccc3n4-c3ccc4ccccc4c3)nc3ccccc23)cc1 ; CC1(C)c2ccccc2-c2cc(N(c3ccc(-c4nc5ccccc5nc4-c4ccccc4)cc3)c3ccc(C4(c5ccccc5)c5ccccc5-c5ccccc54)cc3)ccc21]

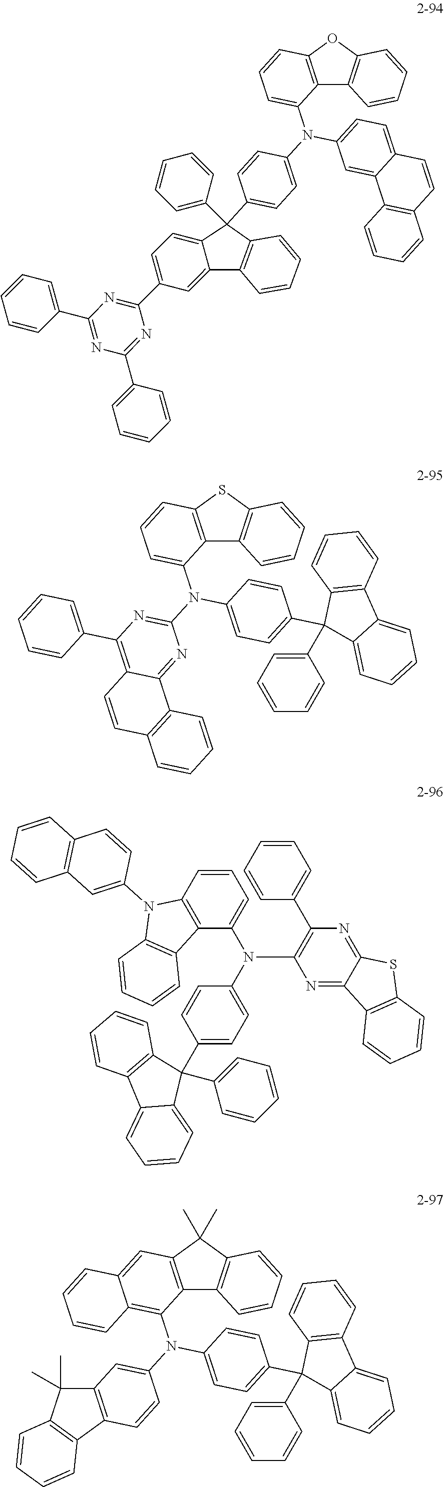 [SMILES: c1ccc(-c2nc(-c3ccccc3)nc(-c3ccc4c(c3)-c3ccccc3C4(c3ccccc3)c3ccc(N(c4ccc5ccc6ccccc6c5c4)c4cccc5oc6ccccc6c45)cc3)n2)cc1 ; c1ccc(-c2nc(N(c3ccc(C4(c5ccccc5)c5ccccc5-c5ccccc54)cc3)c3cccc4sc5ccccc5c34)nc3c2ccc2ccccc23)cc1 ; c1ccc(-c2nc3sc4ccccc4c3nc2N(c2ccc(C3(c4ccccc4)c4ccccc4-c4ccccc43)cc2)c2cccc3c2c2ccccc2n3-c2ccc3ccccc3c2)cc1 ; CC1(C)c2ccccc2-c2ccc(N(c3ccc(C4(c5ccccc5)c5ccccc5-c5ccccc54)cc3)c3c4c(cc5ccccc35)C(C)(C)c3ccccc3-4)cc21]

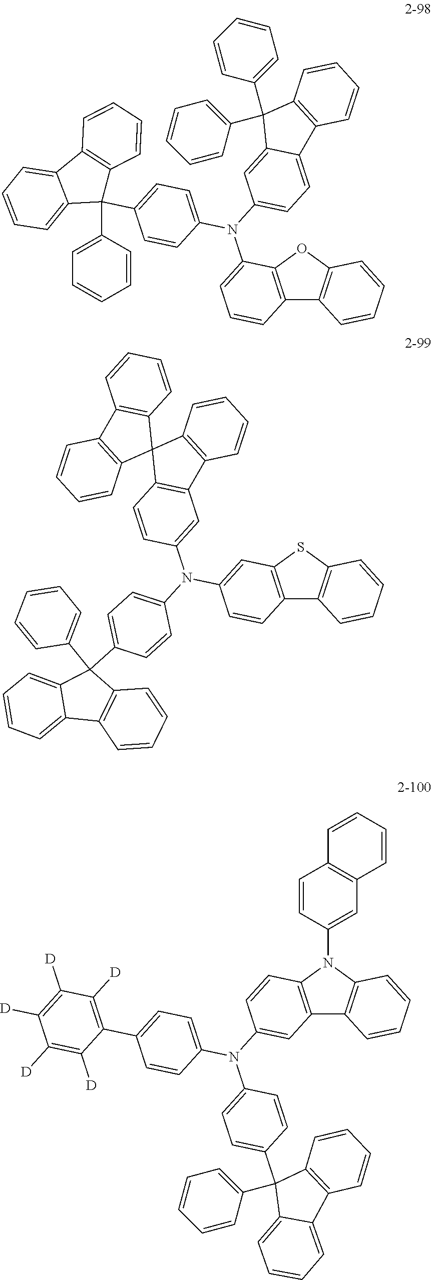 [SMILES: c1ccc(C2(c3ccc(N(c4ccc5c(c4)C(c4ccccc4)(c4ccccc4)c4ccccc4-5)c4cccc5c4oc4ccccc45)cc3)c3ccccc3-c3ccccc32)cc1 ; c1ccc(C2(c3ccc(N(c4ccc5c(c4)-c4ccccc4C54c5ccccc5-c5ccccc54)c4ccc5c(c4)sc4ccccc45)cc3)c3ccccc3-c3ccccc32)cc1 ; [2H]c1c([2H])c([2H])c(-c2ccc(N(c3ccc(C4(c5ccccc5)c5ccccc5-c5ccccc54)cc3)c3ccc4c(c3)c3ccccc3n4-c3ccc4ccccc4c3)cc2)c([2H])c1[2H]]

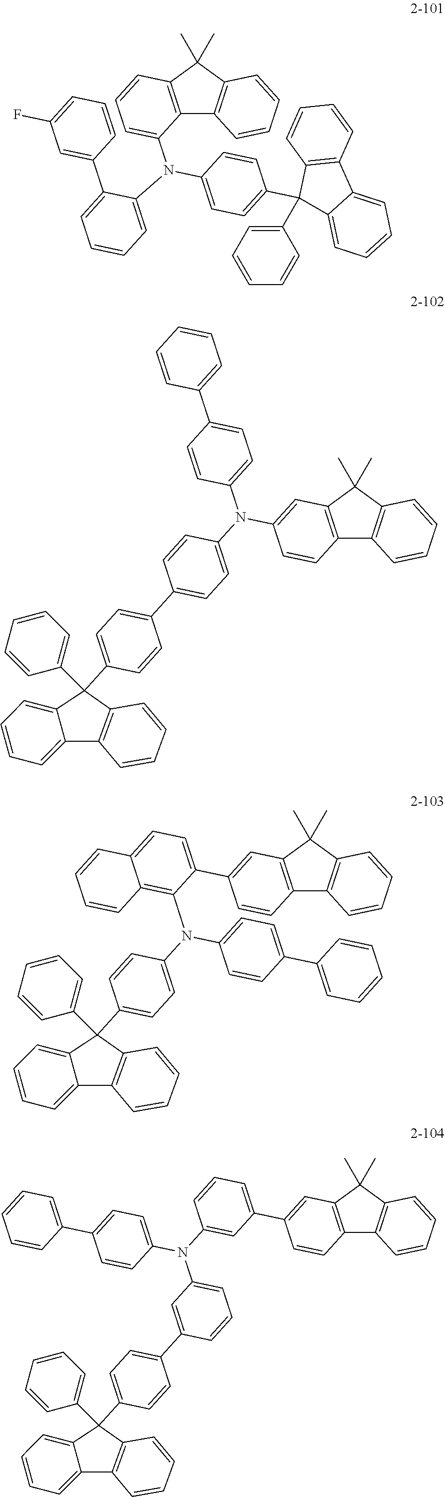 [SMILES: CC1(C)c2ccccc2-c2c(N(c3ccc(C4(c5ccccc5)c5ccccc5-c5ccccc54)cc3)c3ccccc3-c3cccc(F)c3)cccc21 ; CC1(C)c2ccccc2-c2ccc(N(c3ccc(-c4ccccc4)cc3)c3ccc(-c4ccc(C5(c6ccccc6)c6ccccc6-c6ccccc65)cc4)cc3)cc21 ; CC1(C)c2ccccc2-c2ccc(-c3ccc4ccccc4c3N(c3ccc(-c4ccccc4)cc3)c3ccc(C4(c5ccccc5)c5ccccc5-c5ccccc54)cc3)cc21 ; CC1(C)c2ccccc2-c2ccc(-c3cccc(N(c4ccc(-c5ccccc5)cc4)c4cccc(-c5ccc(C6(c7ccccc7)c7ccccc7-c7ccccc76)cc5)c4)c3)cc21]

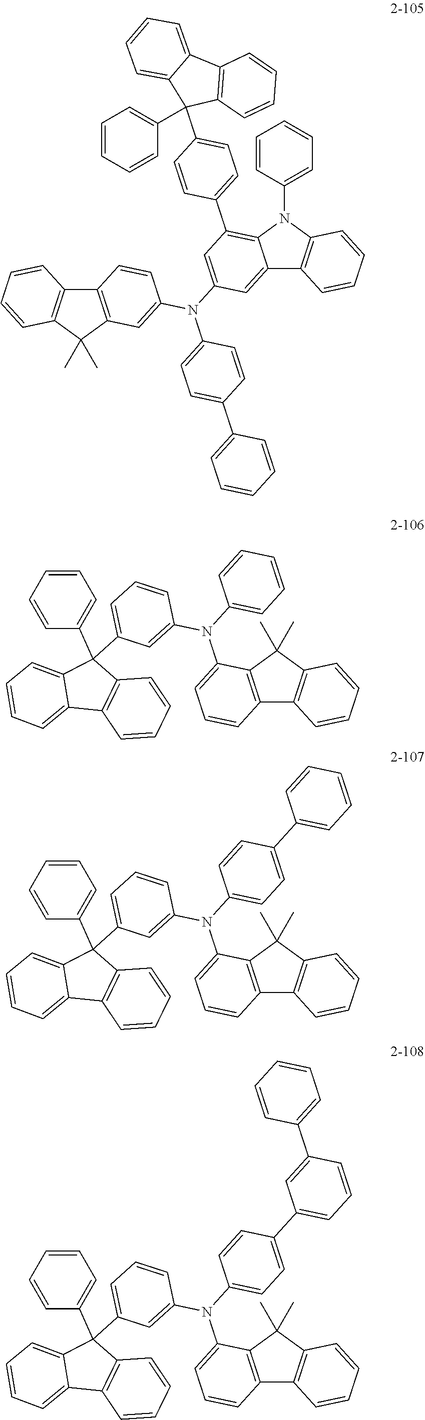

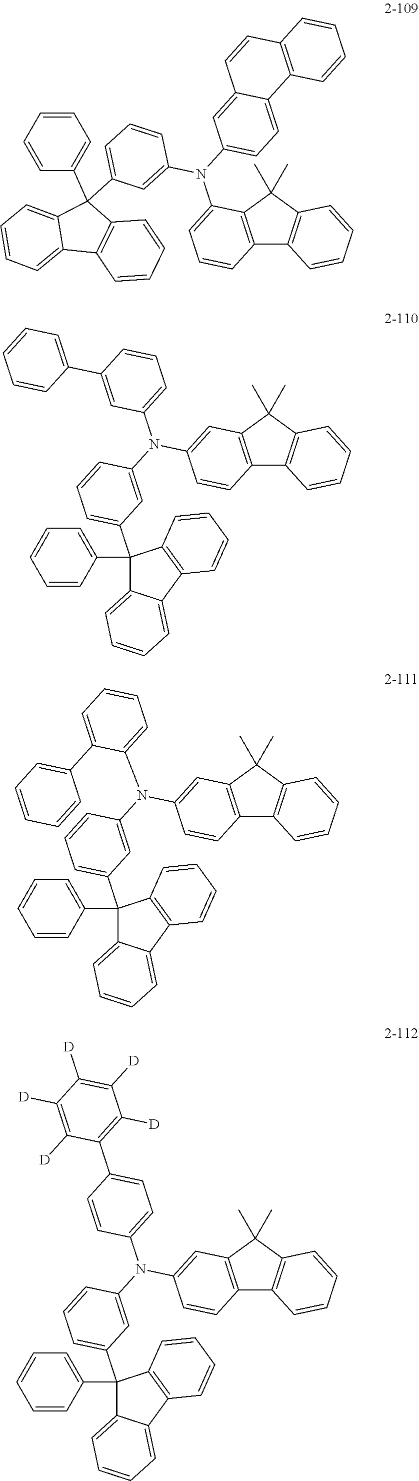

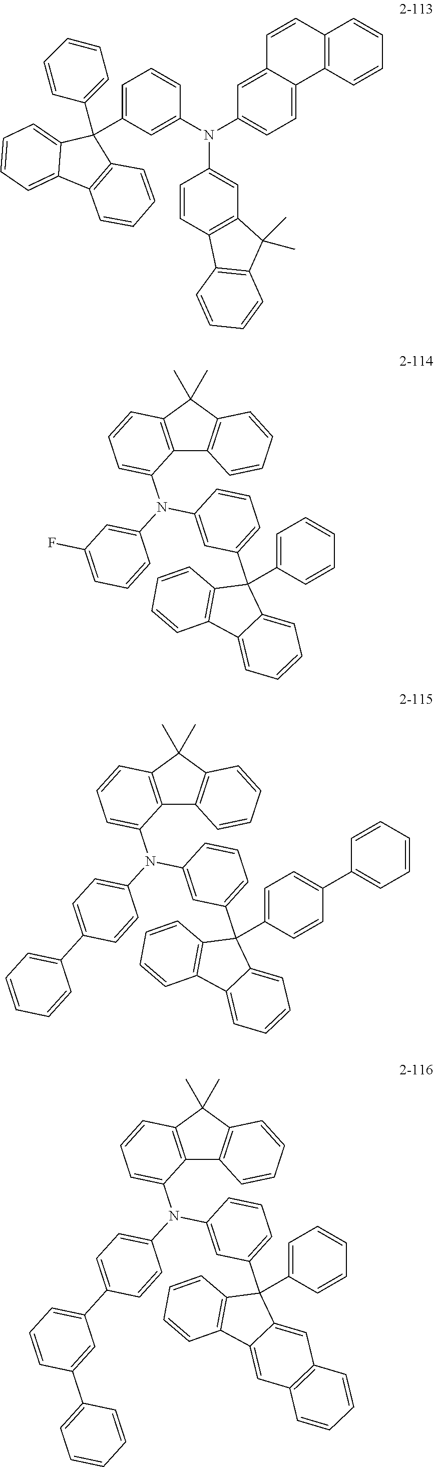

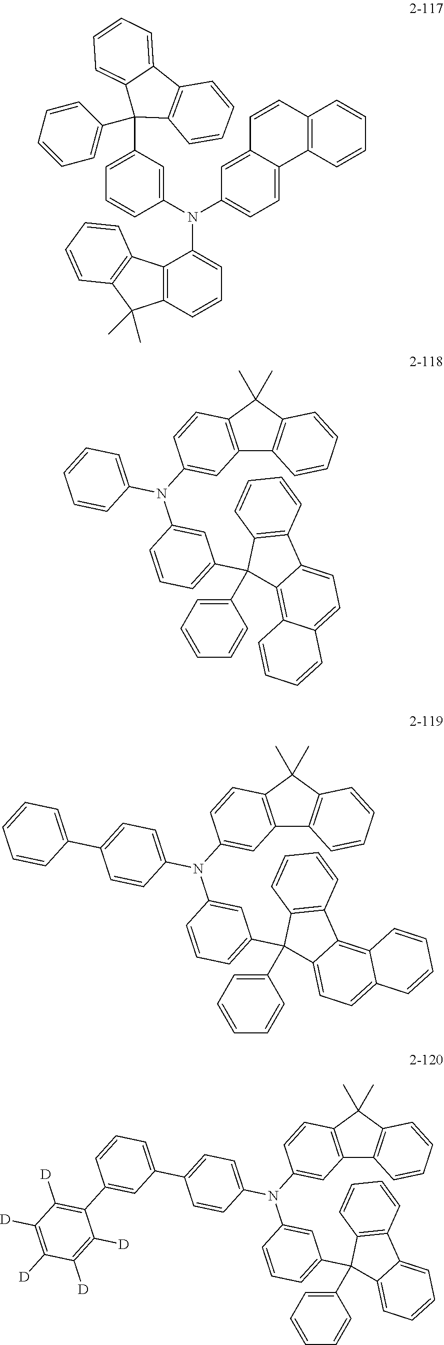 [SMILES: CC1(C)c2ccccc2-c2c(N(c3cccc(C4(c5ccccc5)c5ccccc5-c5ccccc54)c3)c3ccc4c(ccc5ccccc54)c3)cccc21 ; CC1(C)c2ccccc2-c2cc(N(c3ccccc3)c3cccc(C4(c5ccccc5)c5ccccc5-c5ccc6ccccc6c54)c3)ccc21 ; CC1(C)c2ccccc2-c2cc(N(c3ccc(-c4ccccc4)cc3)c3cccc(C4(c5ccccc5)c5ccccc5-c5c4ccc4ccccc54)c3)ccc21 ; [2H]c1c([2H])c([2H])c(-c2cccc(-c3ccc(N(c4cccc(C5(c6ccccc6)c6ccccc6-c6ccccc65)c4)c4ccc5c(c4)-c4ccccc4C5(C)C)cc3)c2)c([2H])c1[2H]]

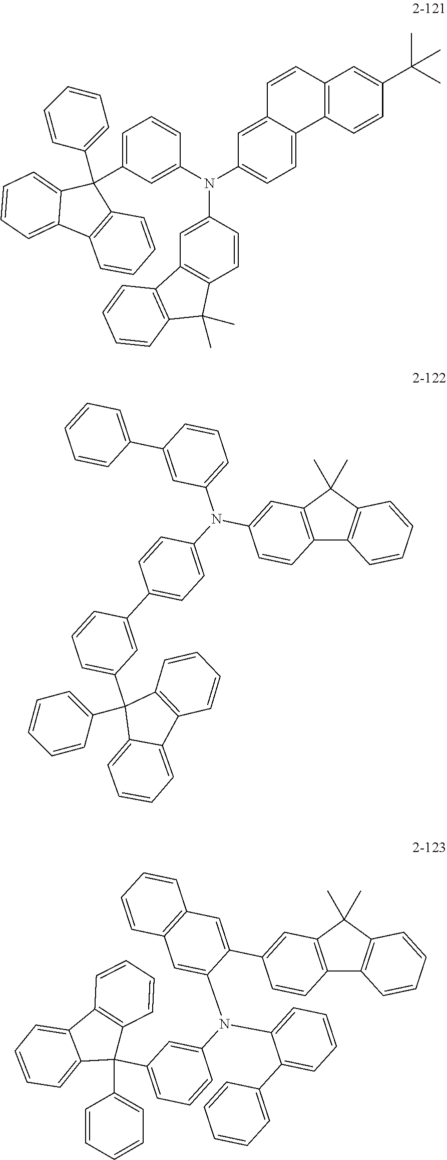 [SMILES: CC(C)(C)c1ccc2c(ccc3cc(N(c4cccc(C5(c6ccccc6)c6ccccc6-c6ccccc65)c4)c4ccc5c(c4)-c4ccccc4C5(C)C)ccc32)c1 ; CC1(C)c2ccccc2-c2ccc(N(c3ccc(-c4cccc(C5(c6ccccc6)c6ccccc6-c6ccccc65)c4)cc3)c3cccc(-c4ccccc4)c3)cc21 ; CC1(C)c2ccccc2-c2ccc(-c3cc4ccccc4cc3N(c3cccc(C4(c5ccccc5)c5ccccc5-c5ccccc54)c3)c3ccccc3-c3ccccc3)cc21]

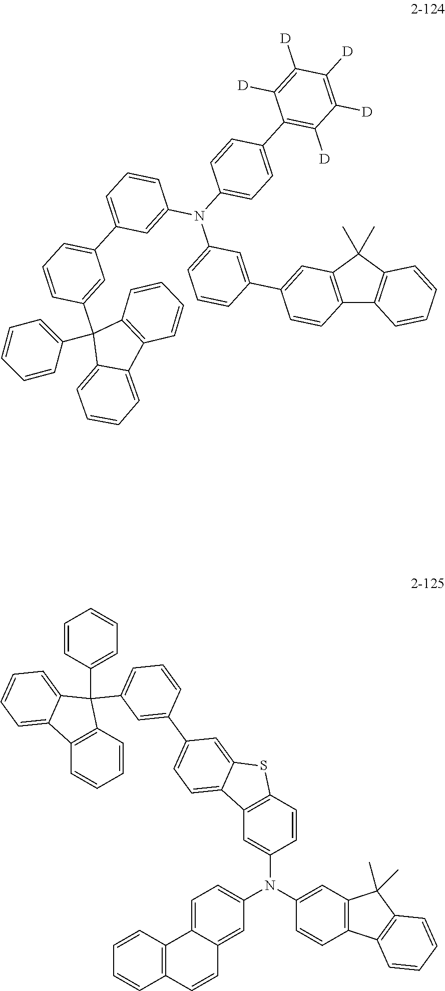 [SMILES: [2H]c1c([2H])c([2H])c(-c2ccc(N(c3cccc(-c4cccc(C5(c6ccccc6)c6ccccc6-c6ccccc65)c4)c3)c3cccc(-c4ccc5c(c4)C(C)(C)c4ccccc4-5)c3)cc2)c([2H])c1[2H] ; CC1(C)c2ccccc2-c2ccc(N(c3ccc4c(ccc5ccccc54)c3)c3ccc4sc5cc(-c6cccc(C7(c8ccccc8)c8ccccc8-c8ccccc87)c6)ccc5c4c3)cc21]

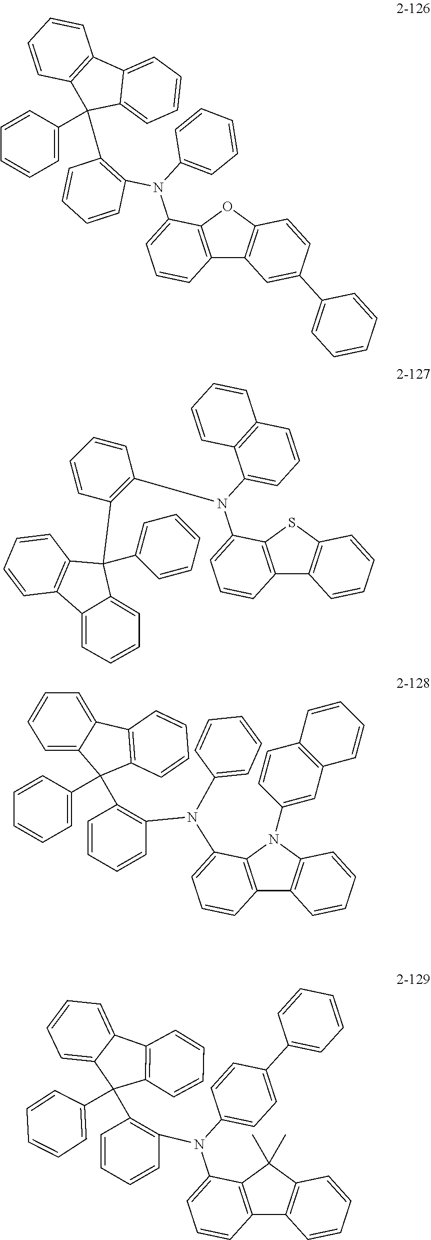

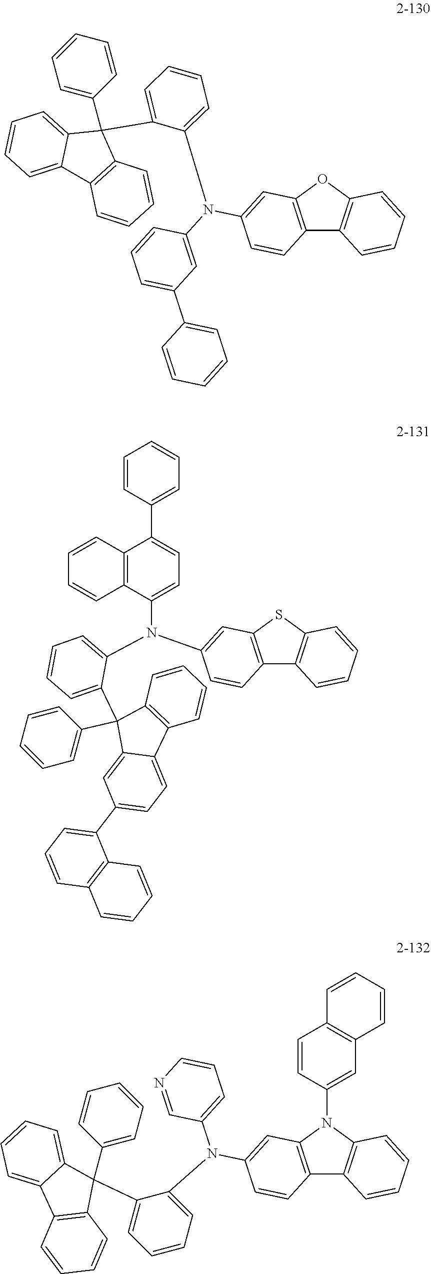 [SMILES: c1ccc(-c2cccc(N(c3ccc4c(c3)oc3ccccc34)c3ccccc3C3(c4ccccc4)c4ccccc4-c4ccccc43)c2)cc1 ; c1ccc(-c2ccc(N(c3ccc4c(c3)sc3ccccc34)c3ccccc3C3(c4ccccc4)c4ccccc4-c4ccc(-c5cccc6ccccc56)cc43)c3ccccc23)cc1 ; c1ccc(C2(c3ccccc3N(c3cccnc3)c3ccc4c5ccccc5n(-c5ccc6ccccc6c5)c4c3)c3ccccc3-c3ccccc32)cc1]

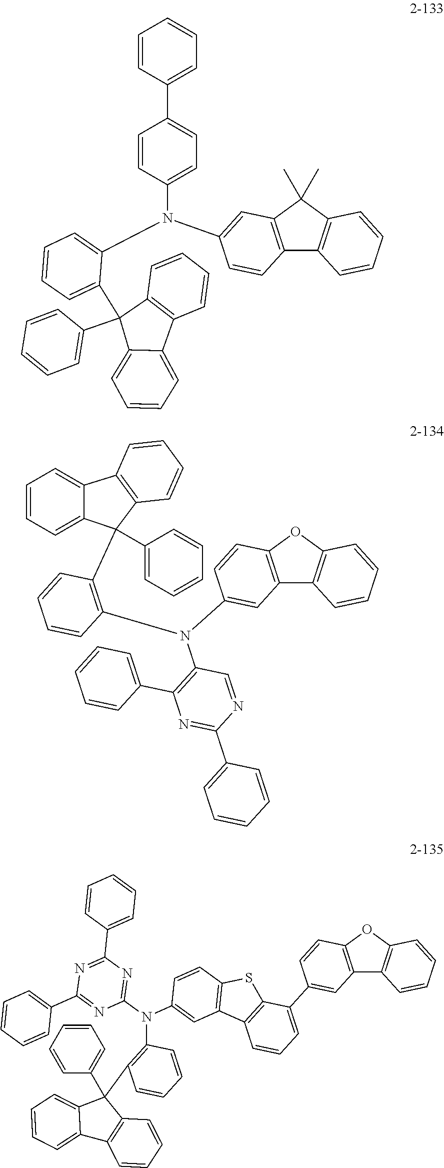

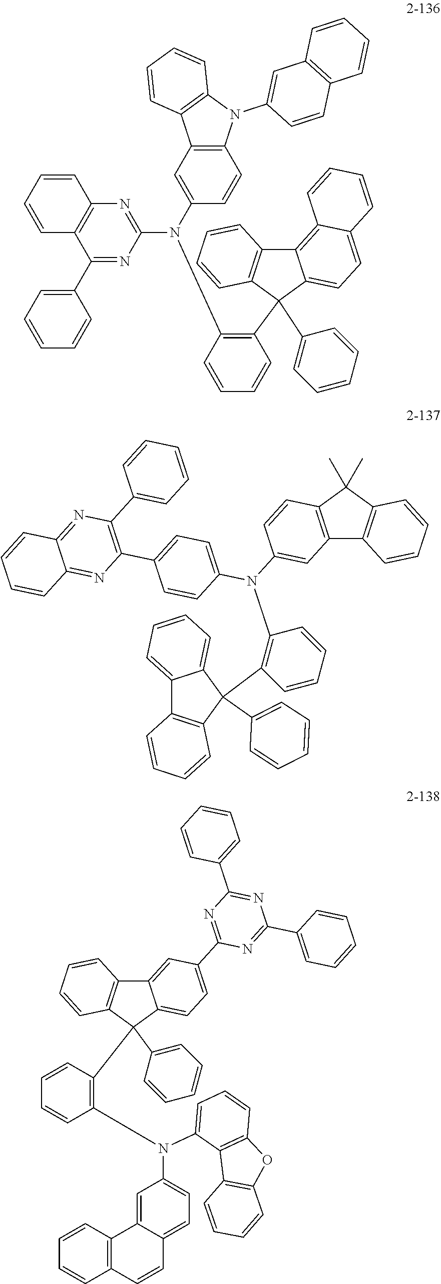

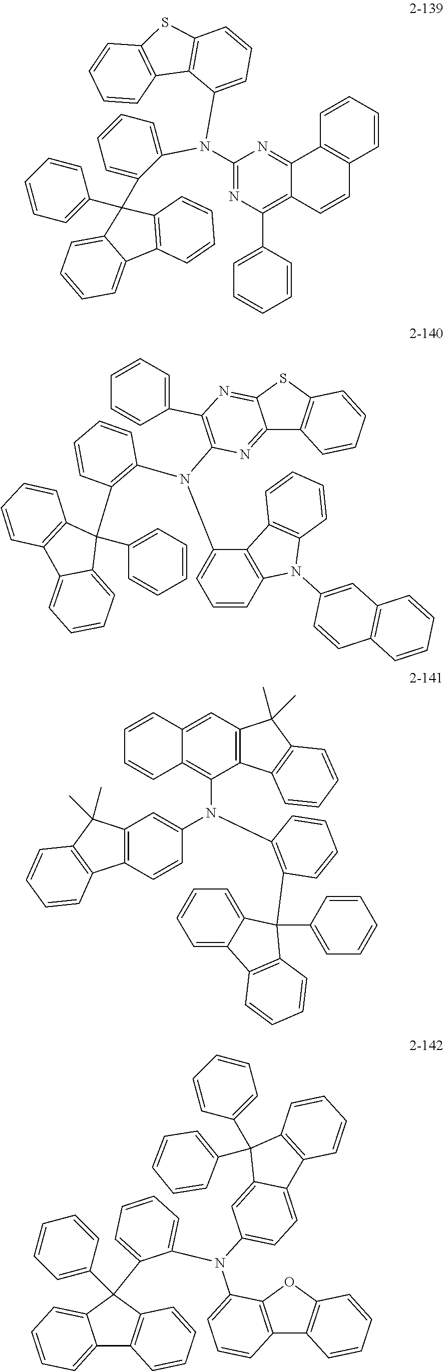

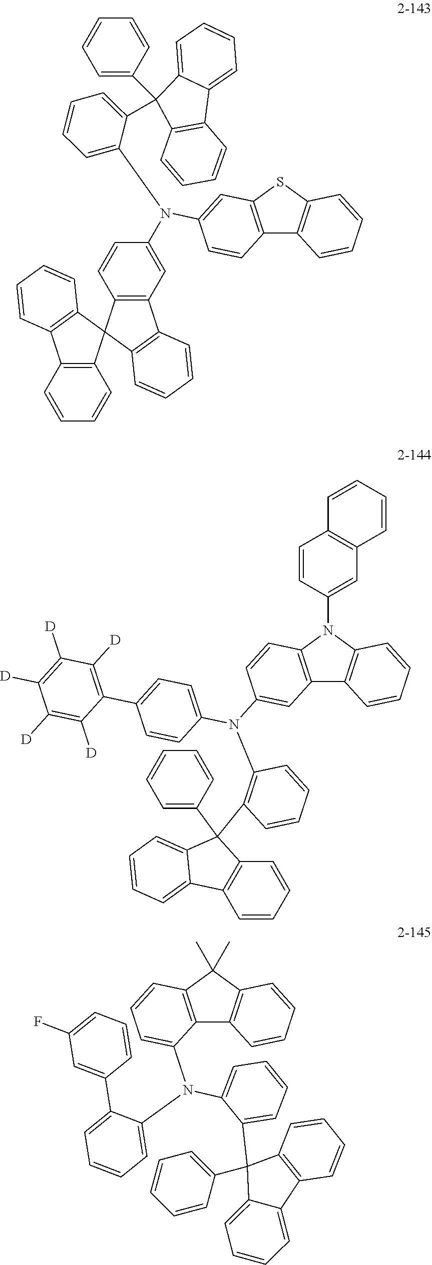

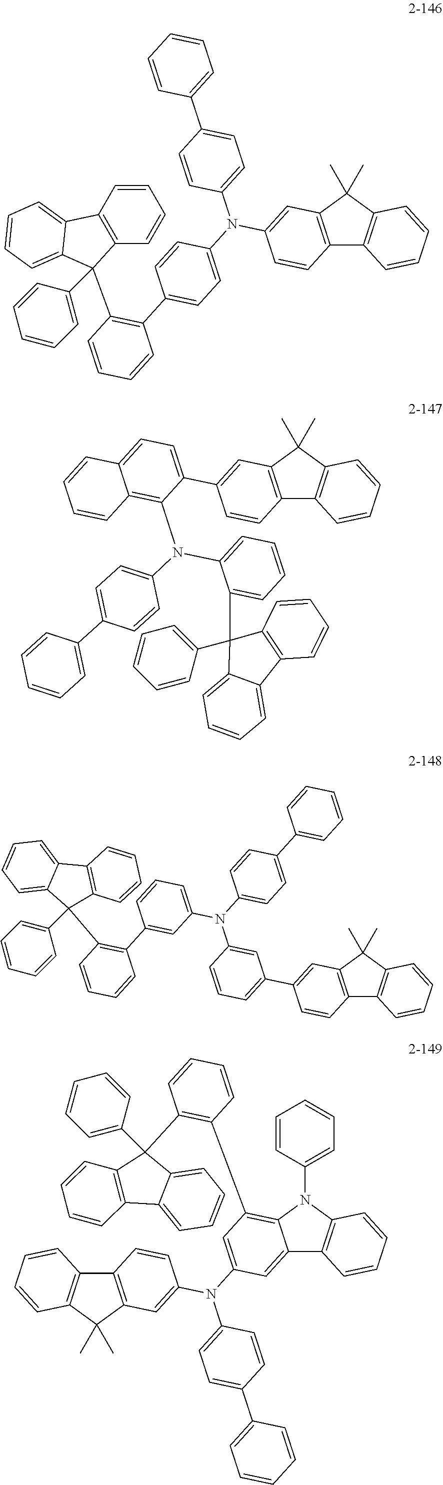 [SMILES: CC1(C)c2ccccc2-c2ccc(N(c3ccc(-c4ccccc4)cc3)c3ccc(-c4ccccc4C4(c5ccccc5)c5ccccc5-c5ccccc54)cc3)cc21 ; CC1(C)c2ccccc2-c2ccc(-c3ccc4ccccc4c3N(c3ccc(-c4ccccc4)cc3)c3ccccc3C3(c4ccccc4)c4ccccc4-c4ccccc43)cc21 ; CC1(C)c2ccccc2-c2ccc(-c3cccc(N(c4ccc(-c5ccccc5)cc4)c4cccc(-c5ccccc5C5(c6ccccc6)c6ccccc6-c6ccccc65)c4)c3)cc21 ; CC1(C)c2ccccc2-c2ccc(N(c3ccc(-c4ccccc4)cc3)c3cc(-c4ccccc4C4(c5ccccc5)c5ccccc5-c5ccccc54)c4c(c3)c3ccccc3n4-c3ccccc3)cc21]

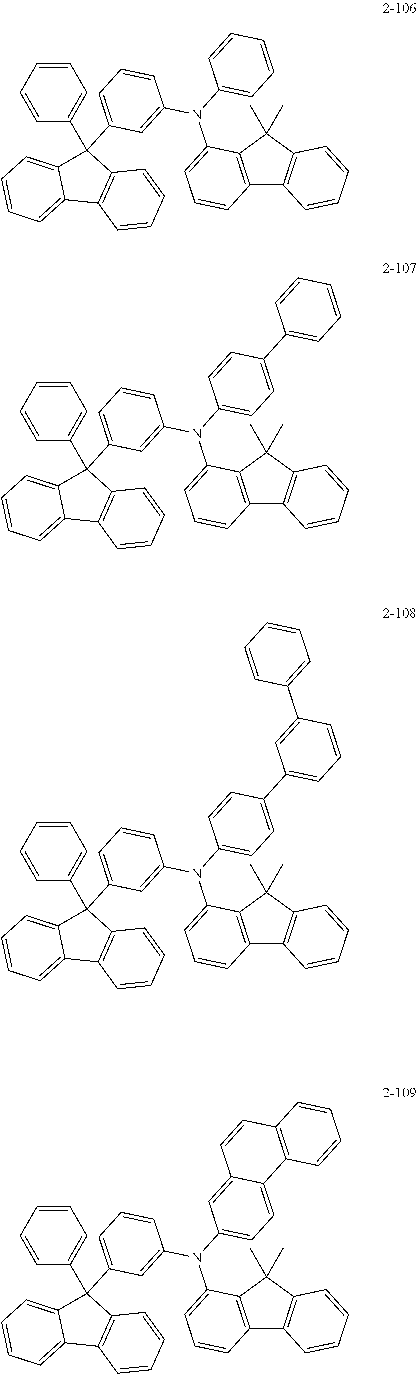 [SMILES: CC1(C)c2ccccc2-c2cccc(N(c3ccccc3)c3cccc(C4(c5ccccc5)c5ccccc5-c5ccccc54)c3)c21 ; CC1(C)c2ccccc2-c2cccc(N(c3ccc(-c4ccccc4)cc3)c3cccc(C4(c5ccccc5)c5ccccc5-c5ccccc54)c3)c21 ; CC1(C)c2ccccc2-c2cccc(N(c3ccc(-c4cccc(-c5ccccc5)c4)cc3)c3cccc(C4(c5ccccc5)c5ccccc5-c5ccccc54)c3)c21 ; CC1(C)c2ccccc2-c2cccc(N(c3cccc(C4(c5ccccc5)c5ccccc5-c5ccccc54)c3)c3ccc4c(ccc5ccccc54)c3)c21]

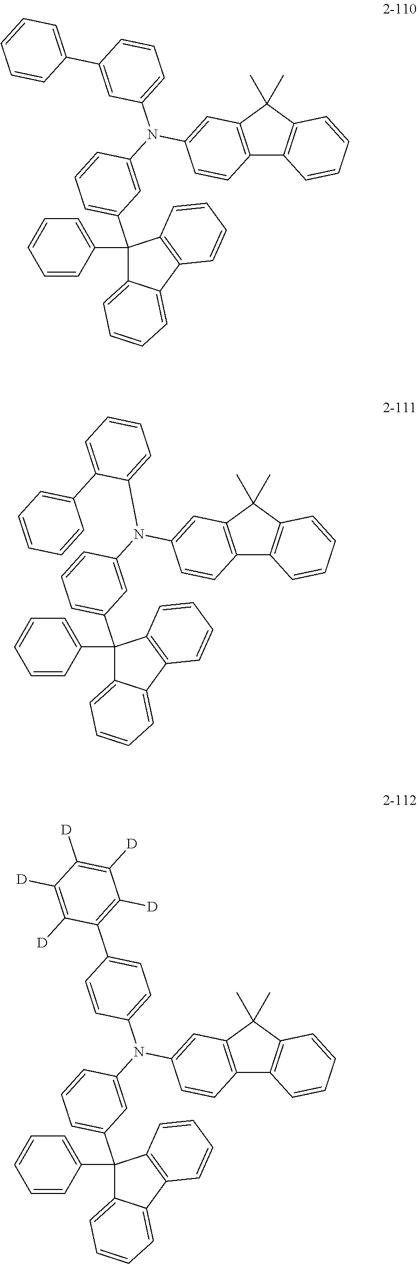 [SMILES: CC1(C)c2ccccc2-c2ccc(N(c3cccc(-c4ccccc4)c3)c3cccc(C4(c5ccccc5)c5ccccc5-c5ccccc54)c3)cc21 ; CC1(C)c2ccccc2-c2ccc(N(c3cccc(C4(c5ccccc5)c5ccccc5-c5ccccc54)c3)c3ccccc3-c3ccccc3)cc21 ; [2H]c1c([2H])c([2H])c(-c2ccc(N(c3cccc(C4(c5ccccc5)c5ccccc5-c5ccccc54)c3)c3ccc4c(c3)C(C)(C)c3ccccc3-4)cc2)c([2H])c1[2H]]

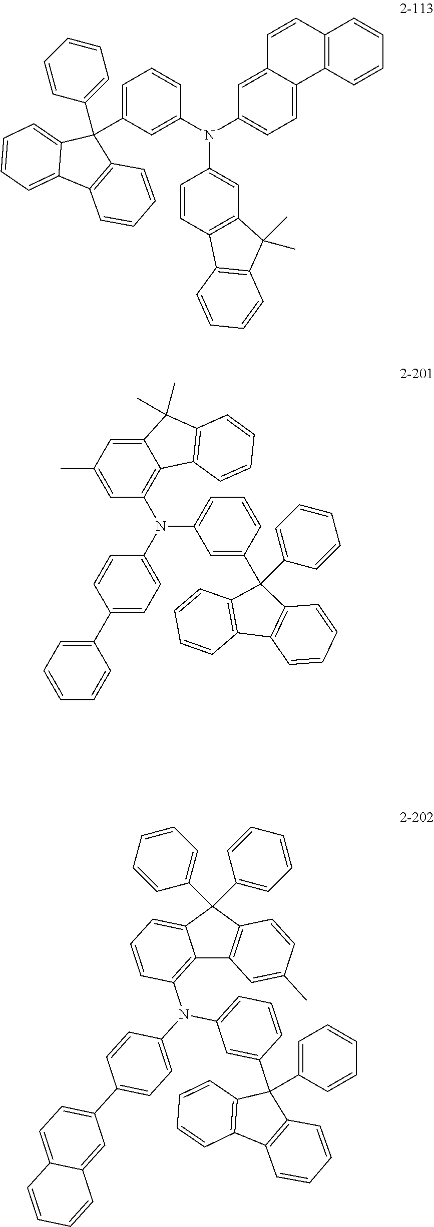 [SMILES: CC1(C)c2ccccc2-c2ccc(N(c3cccc(C4(c5ccccc5)c5ccccc5-c5ccccc54)c3)c3ccc4c(ccc5ccccc54)c3)cc21 ; Cc1cc(N(c2ccc(-c3ccccc3)cc2)c2cccc(C3(c4ccccc4)c4ccccc4-c4ccccc43)c2)c2c(c1)C(C)(C)c1ccccc1-2 ; Cc1ccc2c(c1)-c1c(N(c3ccc(-c4ccc5ccccc5c4)cc3)c3cccc(C4(c5ccccc5)c5ccccc5-c5ccccc54)c3)cccc1C2(c1ccccc1)c1ccccc1]

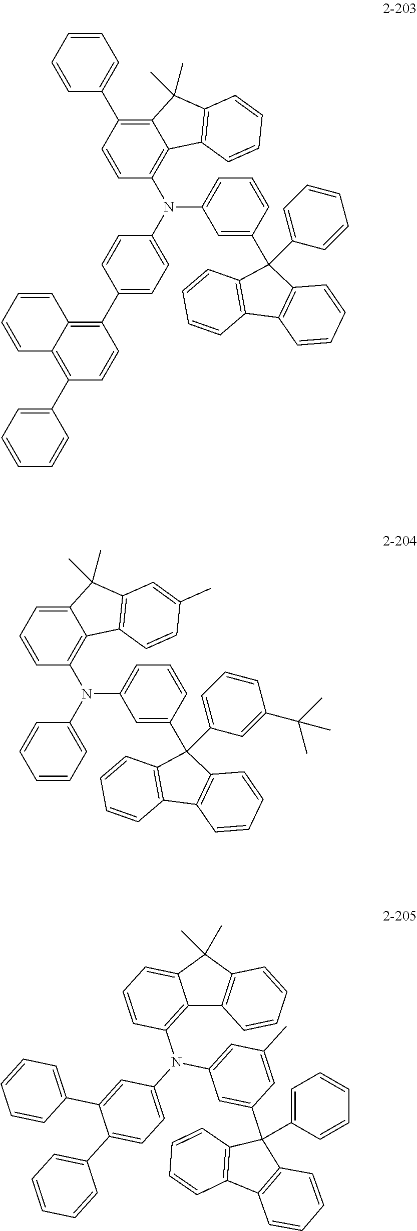 [SMILES: CC1(C)c2ccccc2-c2c(N(c3ccc(-c4ccc(-c5ccccc5)c5ccccc45)cc3)c3cccc(C4(c5ccccc5)c5ccccc5-c5ccccc54)c3)ccc(-c3ccccc3)c21 ; Cc1ccc2c(c1)C(C)(C)c1cccc(N(c3ccccc3)c3cccc(C4(c5cccc(C(C)(C)C)c5)c5ccccc5-c5ccccc54)c3)c1-2 ; Cc1cc(N(c2ccc(-c3ccccc3)c(-c3ccccc3)c2)c2cccc3c2-c2ccccc2C3(C)C)cc(C2(c3ccccc3)c3ccccc3-c3ccccc32)c1]

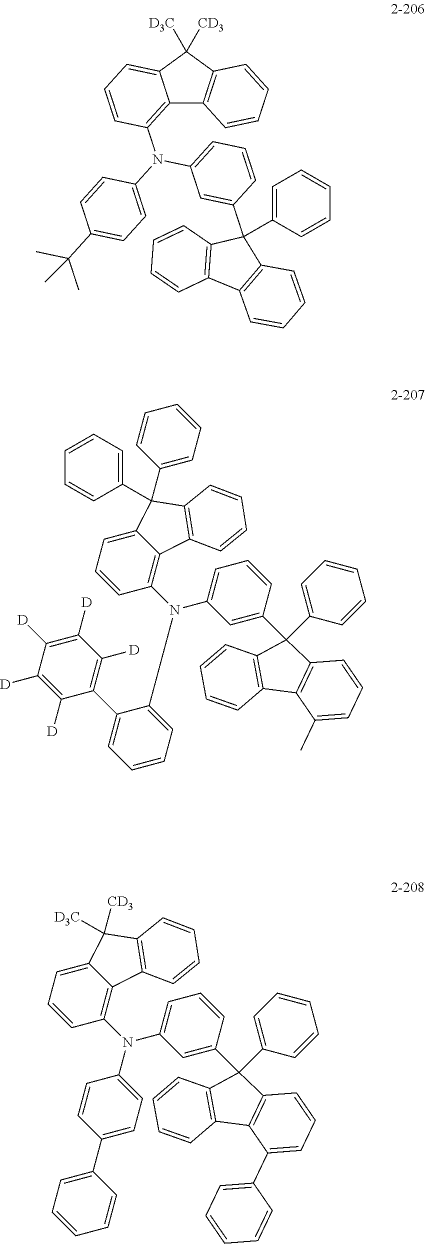 [SMILES: [2H]C([2H])([2H])C1(C([2H])([2H])[2H])c2ccccc2-c2c(N(c3ccc(C(C)(C)C)cc3)c3cccc(C4(c5ccccc5)c5ccccc5-c5ccccc54)c3)cccc21 ; [2H]c1c([2H])c([2H])c(-c2ccccc2N(c2cccc(C3(c4ccccc4)c4ccccc4-c4c(C)cccc43)c2)c2cccc3c2-c2ccccc2C3(c2ccccc2)c2ccccc2)c([2H])c1[2H] ; [2H]C([2H])([2H])C1(C([2H])([2H])[2H])c2ccccc2-c2c(N(c3ccc(-c4ccccc4)cc3)c3cccc(C4(c5ccccc5)c5ccccc5-c5c(-c6ccccc6)cccc54)c3)cccc21]

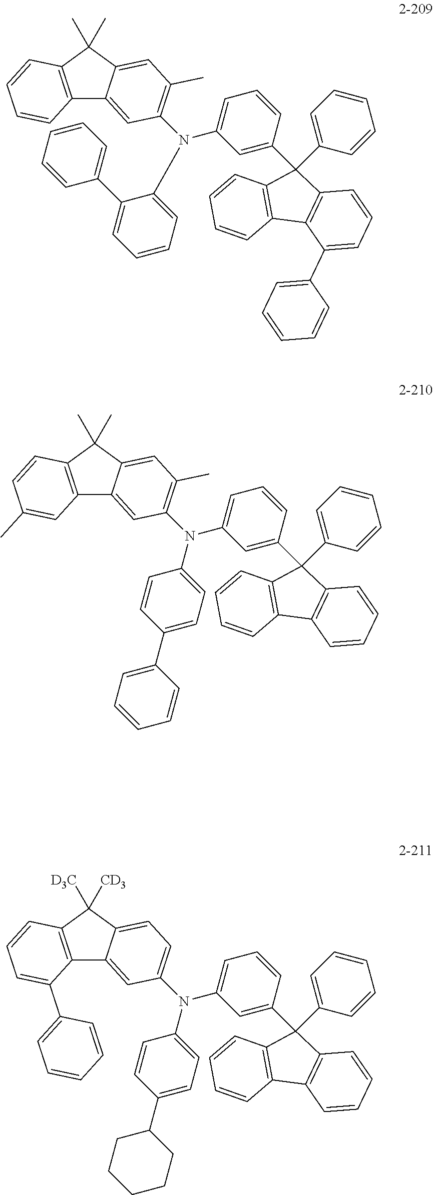 [SMILES: Cc1cc2c(cc1N(c1cccc(C3(c4ccccc4)c4ccccc4-c4c(-c5ccccc5)cccc43)c1)c1ccccc1-c1ccccc1)-c1ccccc1C2(C)C ; Cc1ccc2c(c1)-c1cc(N(c3ccc(-c4ccccc4)cc3)c3cccc(C4(c5ccccc5)c5ccccc5-c5ccccc54)c3)c(C)cc1C2(C)C ; [2H]C([2H])([2H])C1(C([2H])([2H])[2H])c2ccc(N(c3ccc(C4CCCCC4)cc3)c3cccc(C4(c5ccccc5)c5ccccc5-c5ccccc54)c3)cc2-c2c(-c3ccccc3)cccc21]

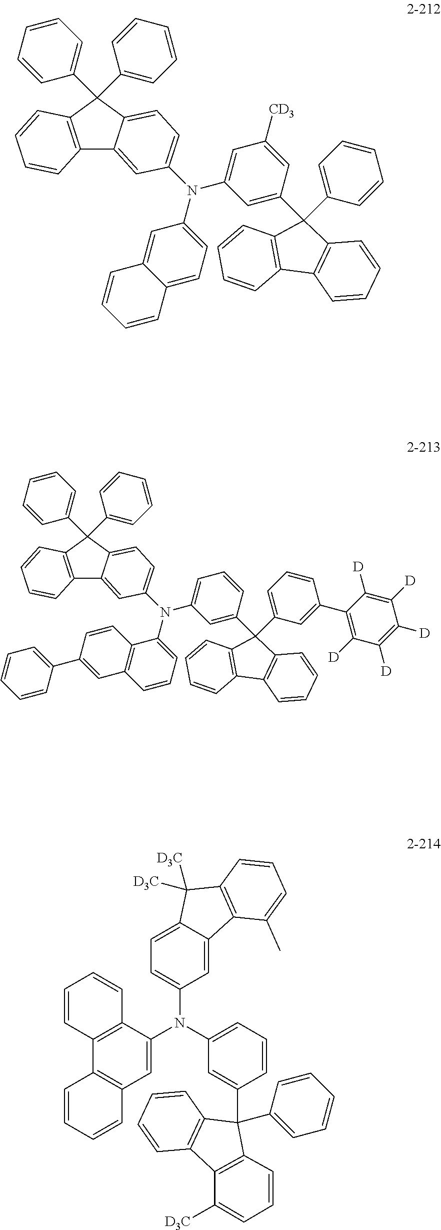 [SMILES: [2H]C([2H])([2H])c1cc(N(c2ccc3c(c2)-c2ccccc2C3(c2ccccc2)c2ccccc2)c2ccc3ccccc3c2)cc(C2(c3ccccc3)c3ccccc3-c3ccccc32)c1 ; [2H]c1c([2H])c([2H])c(-c2cccc(C3(c4cccc(N(c5ccc6c(c5)-c5ccccc5C6(c5ccccc5)c5ccccc5)c5cccc6cc(-c7ccccc7)ccc56)c4)c4ccccc4-c4ccccc43)c2)c([2H])c1[2H] ; [2H]C([2H])([2H])c1cccc2c1-c1ccccc1C2(c1ccccc1)c1cccc(N(c2ccc3c(c2)-c2c(C)cccc2C3(C([2H])([2H])[2H])C([2H])([2H])[2H])c2cc3ccccc3c3ccccc23)c1]

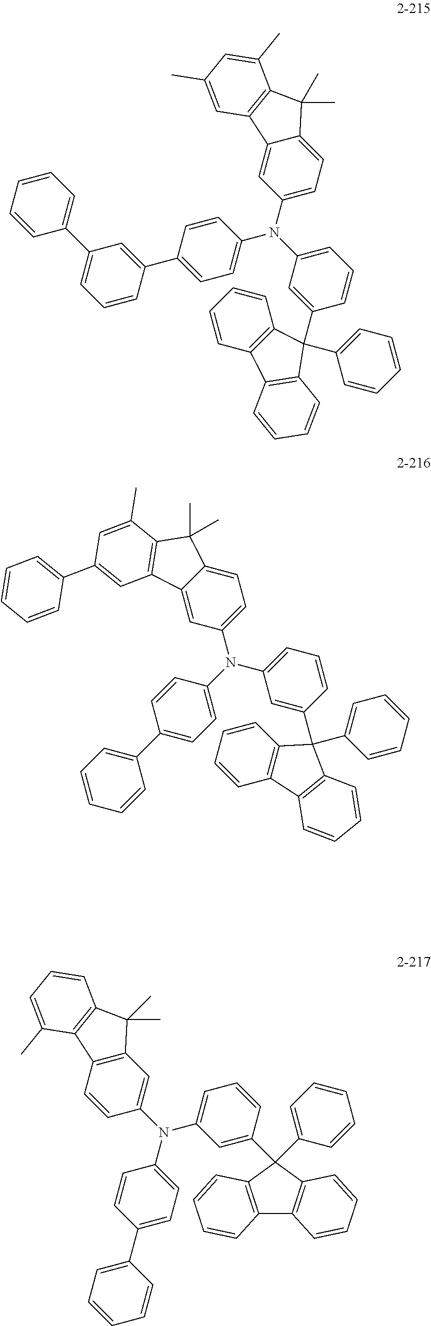 [SMILES: Cc1cc(C)c2c(c1)-c1cc(N(c3ccc(-c4cccc(-c5ccccc5)c4)cc3)c3cccc(C4(c5ccccc5)c5ccccc5-c5ccccc54)c3)ccc1C2(C)C ; Cc1cc(-c2ccccc2)cc2c1C(C)(C)c1ccc(N(c3ccc(-c4ccccc4)cc3)c3cccc(C4(c5ccccc5)c5ccccc5-c5ccccc54)c3)cc1-2 ; Cc1cccc2c1-c1ccc(N(c3ccc(-c4ccccc4)cc3)c3cccc(C4(c5ccccc5)c5ccccc5-c5ccccc54)c3)cc1C2(C)C]

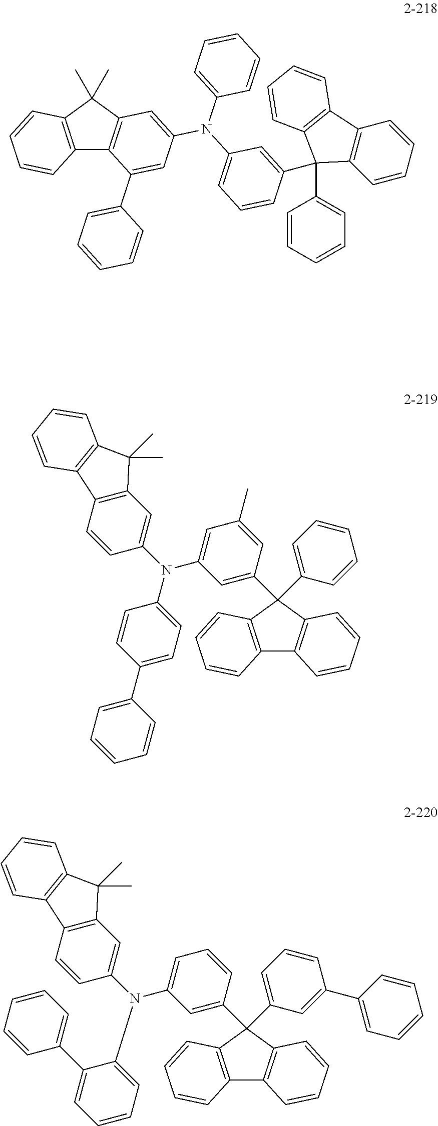 [SMILES: CC1(C)c2ccccc2-c2c(-c3ccccc3)cc(N(c3ccccc3)c3cccc(C4(c5ccccc5)c5ccccc5-c5ccccc54)c3)cc21 ; Cc1cc(N(c2ccc(-c3ccccc3)cc2)c2ccc3c(c2)C(C)(C)c2ccccc2-3)cc(C2(c3ccccc3)c3ccccc3-c3ccccc32)c1 ; CC1(C)c2ccccc2-c2ccc(N(c3cccc(C4(c5cccc(-c6ccccc6)c5)c5ccccc5-c5ccccc54)c3)c3ccccc3-c3ccccc3)cc21]

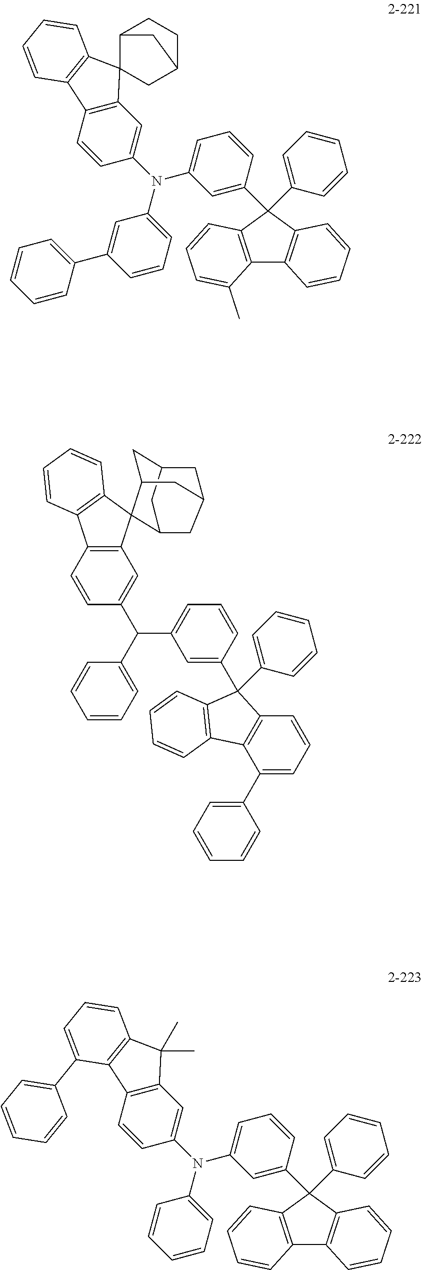

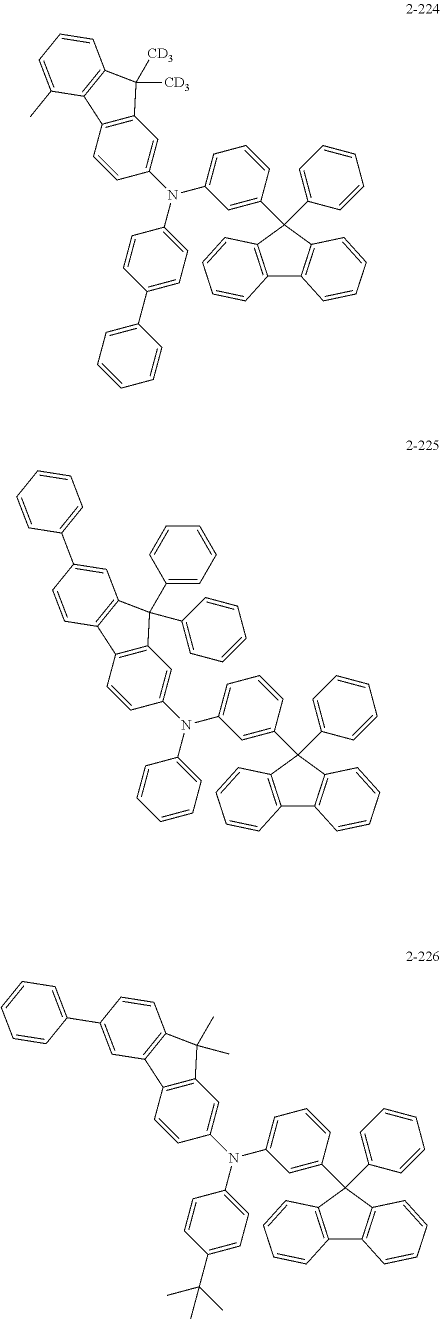 [SMILES: [2H]C([2H])([2H])C1(C([2H])([2H])[2H])c2cc(N(c3ccc(-c4ccccc4)cc3)c3cccc(C4(c5ccccc5)c5ccccc5-c5ccccc54)c3)ccc2-c2c(C)cccc21 ; c1ccc(-c2ccc3c(c2)C(c2ccccc2)(c2ccccc2)c2cc(N(c4ccccc4)c4cccc(C5(c6ccccc6)c6ccccc6-c6ccccc65)c4)ccc2-3)cc1 ; CC(C)(C)c1ccc(N(c2cccc(C3(c4ccccc4)c4ccccc4-c4ccccc43)c2)c2ccc3c(c2)C(C)(C)c2ccc(-c4ccccc4)cc2-3)cc1]

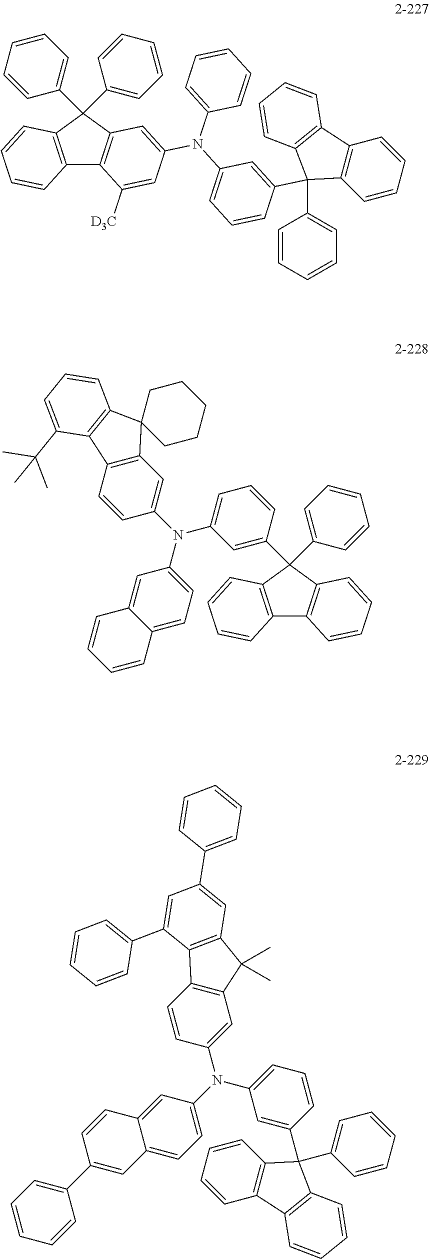 [SMILES: [2H]C([2H])([2H])c1cc(N(c2ccccc2)c2cccc(C3(c4ccccc4)c4ccccc4-c4ccccc43)c2)cc2c1-c1ccccc1C2(c1ccccc1)c1ccccc1 ; CC(C)(C)c1cccc2c1-c1ccc(N(c3cccc(C4(c5ccccc5)c5ccccc5-c5ccccc54)c3)c3ccc4ccccc4c3)cc1C21CCCCC1 ; CC1(C)c2cc(N(c3cccc(C4(c5ccccc5)c5ccccc5-c5ccccc54)c3)c3ccc4cc(-c5ccccc5)ccc4c3)ccc2-c2c(-c3ccccc3)cc(-c3ccccc3)cc21]

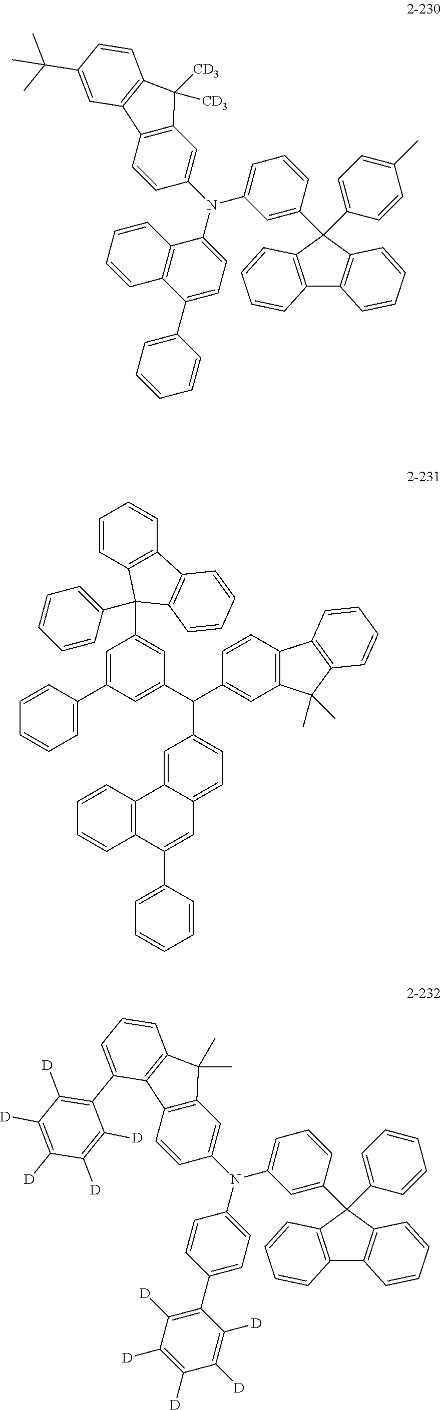

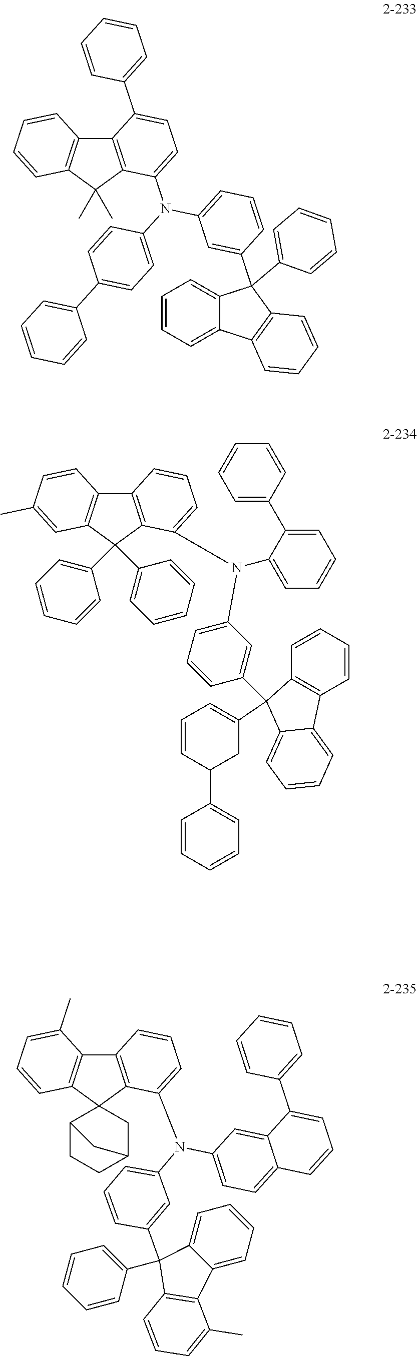 [SMILES: CC1(C)c2ccccc2-c2c(-c3ccccc3)ccc(N(c3ccc(-c4ccccc4)cc3)c3cccc(C4(c5ccccc5)c5ccccc5-c5ccccc54)c3)c21 ; Cc1ccc2c(c1)C(c1ccccc1)(c1ccccc1)c1c-2cccc1N(c1cccc(C2(C3=CC=CC(c4ccccc4)C3)c3ccccc3-c3ccccc32)c1)c1ccccc1-c1ccccc1 ; Cc1cccc2c1-c1ccccc1C2(c1ccccc1)c1cccc(N(c2ccc3cccc(-c4ccccc4)c3c2)c2cccc3c2C2(CC4CCC2C4)c2cccc(C)c2-3)c1]

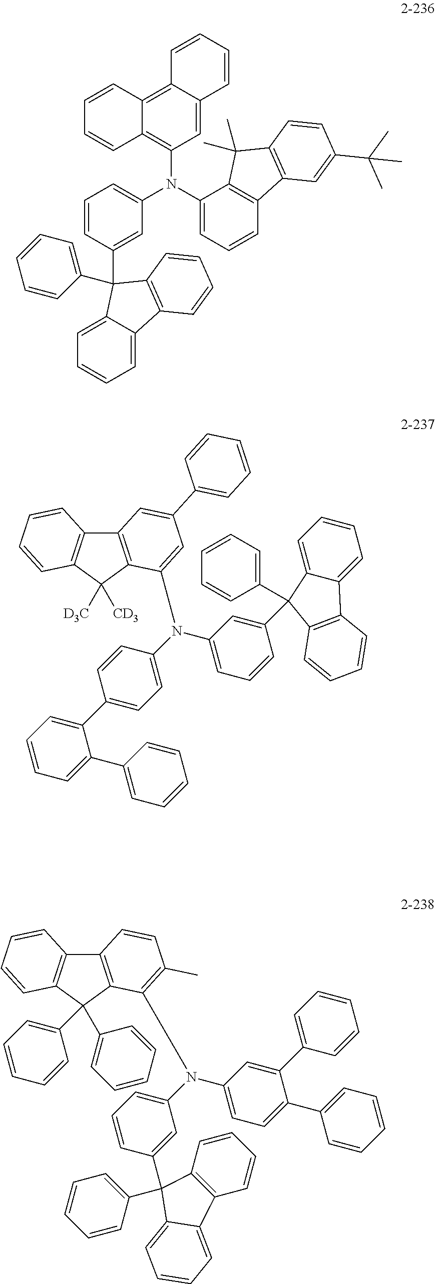 [SMILES: CC(C)(C)c1ccc2c(c1)-c1cccc(N(c3cccc(C4(c5ccccc5)c5ccccc5-c5ccccc54)c3)c3cc4ccccc4c4ccccc34)c1C2(C)C ; [2H]C([2H])([2H])C1(C([2H])([2H])[2H])c2ccccc2-c2cc(-c3ccccc3)cc(N(c3ccc(-c4ccccc4-c4ccccc4)cc3)c3cccc(C4(c5ccccc5)c5ccccc5-c5ccccc54)c3)c21 ; Cc1ccc2c(c1N(c1cccc(C3(c4ccccc4)c4ccccc4-c4ccccc43)c1)c1ccc(-c3ccccc3)c(-c3ccccc3)c1)C(c1ccccc1)(c1ccccc1)c1ccccc1-2]

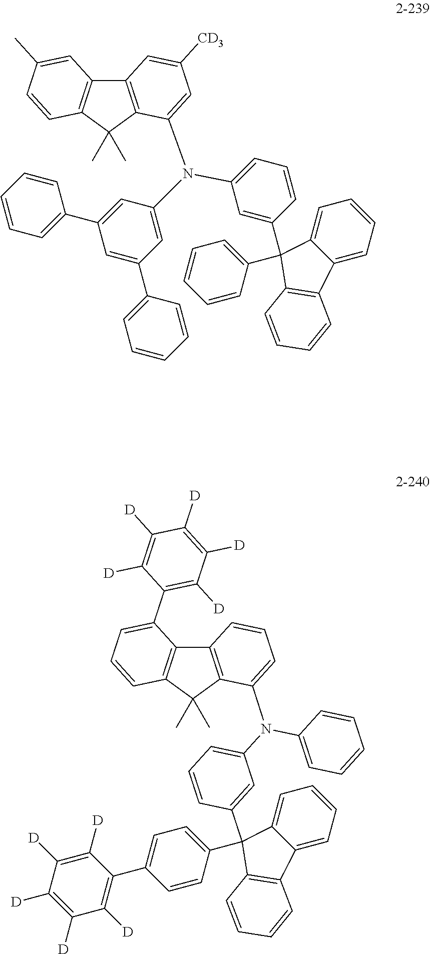 [SMILES: [2H]C([2H])([2H])c1cc2c(c(N(c3cc(-c4ccccc4)cc(-c4ccccc4)c3)c3cccc(C4(c5ccccc5)c5ccccc5-c5ccccc54)c3)c1)C(C)(C)c1ccc(C)cc1-2 ; [2H]c1c([2H])c([2H])c(-c2ccc(C3(c4cccc(N(c5ccccc5)c5cccc6c5C(C)(C)c5cccc(-c7c([2H])c([2H])c([2H])c([2H])c7[2H])c5-6)c4)c4ccccc4-c4ccccc43)cc2)c([2H])c1[2H]]

And more preferably, the present invention provides a compound represented by Formula (32).

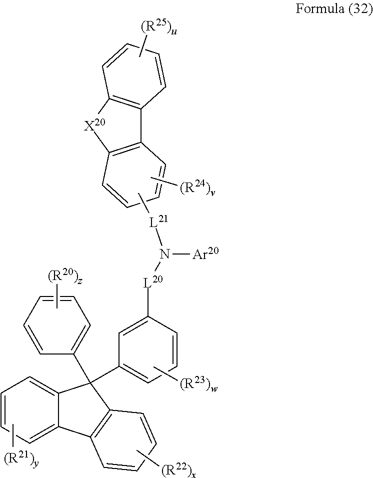

in Formula (32),

1) R²⁰, R²¹, R²², R²³, R²⁴ and R²⁵ are each independently selected from the group consisting of hydrogen; deuterium; a C₆-C₃₀ aryl group; a C₁-C₂₀ alkyl group; and plurality of R²⁰, plurality of R²¹, plurality of R²², plurality of R²³, and plurality of R²⁴ can not be bonded to each other to form a ring,

3) v is an integer of 0 to 3, u, w, x and y are each independently an integer of 0 to 4, z is an integer of 0 to 5,

4) L²⁰ and L²¹ are a single bond,

5) Ar²⁰ is a C₆-C₃₀ aryl group,

X²⁰ is CR′R″,

R′ and R″ are each independently selected from the group of a C₁-C₃₀ alkyl

group; a C₆-C₃₀ aryl group; and R′ and R″ may be bonded to each other to form a spiro wherein, the aryl group and alkyl group may be substituted with one or more substituents selected from the group consisting of deuterium; halogen; a silane group; siloxane group; boron group; germanium group; cyano group; nitro group; a

C₁-C₂₀ alkylthio group; C₁-C₂₀ alkoxy group; C₁-C₂₀ alkyl group; C₂-C₂₀ alkenyl group; C₂-C₂₀ alkynyl group; C₆-C₂₀ aryl group; C₆-C₂₀ aryl group substituted with deuterium; a fluorenyl group; C₂-C₂₀ heterocyclic group; C₃-C₂₀ cycloalkyl group; C7-C20 arylalkyl group and C₈-C₂₀ arylalkenyl group, wherein the substituents may be bonded to each other to form a saturated or unsaturated ring, wherein the term ‘ring’ means a C₃-C₆₀ aliphatic ring or a C₆-C₆₀ aromatic ring or a C₂-C₆₀ heterocyclic group or a fused ring formed by the combination thereof.

Also, the compound represented by Formula 32 may be represented by the following Formula 38 or Formula 39.

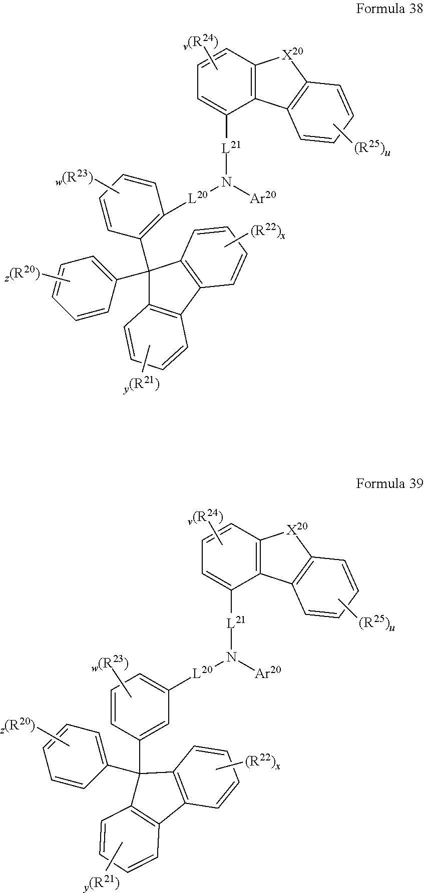

in Formulas (38) to (39),

R²⁰, R²¹, R²², R²³, R²⁴, R²⁵, L²⁰, L²¹, Ar²⁰ and X²⁰, u, v, w, x, y and z are the same as defined above.

The present invention provides a compound wherein u+v+w+x+y+z≥1 and, R²⁰ to R²⁵ are represented by any one of a C₆-C₃₀ aryl group; or a C₁-C₂₀ alkyl group. and more preferably v+u≥1.

The present invention provides a compound wherein Ar²⁰ is represented by any one of Formulas Ar-1 to Ar-8.

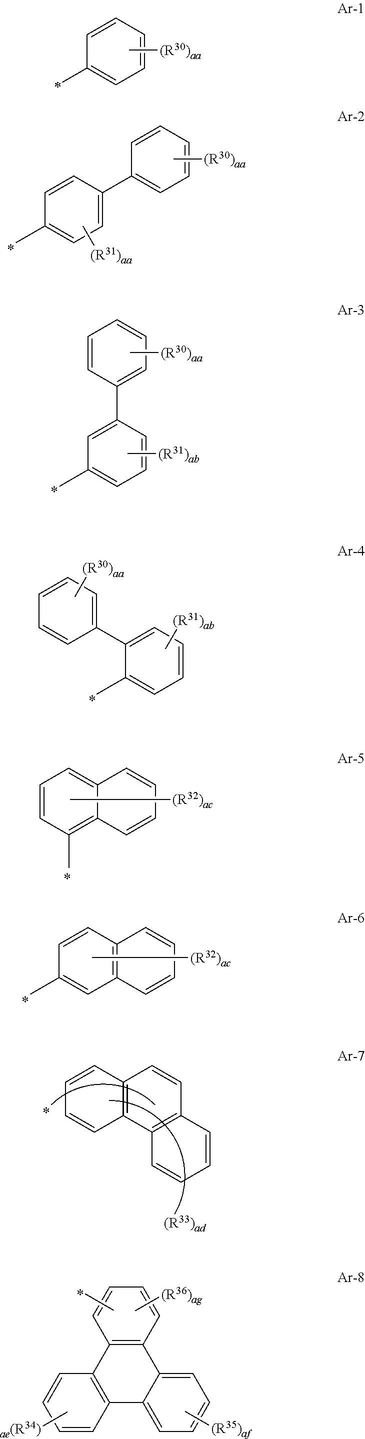

In Formula Ar-1 to Ar-8,

1) R³⁰, R³¹, R³², R³³, R³⁴, R³⁵ and R³⁶ are each independently selected from the group consisting of hydrogen; deuterium; C₁-C₃₀ alkyl group; and a C₆-C₂₀ aryl group; and each as plurality are the same as or different from each other, and a plurality of R³⁰ or a plurality of R³¹ or a plurality of R³² or a plurality of R³³ or or a plurality of R³⁴ or a plurality of R³⁵ or a plurality of R³⁶ or a plurality of R³⁷ or a plurality of R³⁸ may be bonded to each other to form an aromatic or a heteroaromatic ring,

2) aa=an integer of 0 to 5; ab is an integer of 0 to 4; ac is an integer of 0 to 7; ad is an integer of 0 to 9; ae, af and ai are each independently an integer of 0 to 4; ag and ah are each independently an integer of 0 to 3.

In the present invention, the compound represented by Formula (32) comprises the following compounds.

In another aspect, the present invention provides an organic electronic element comprising a compound represented by Formula 32.

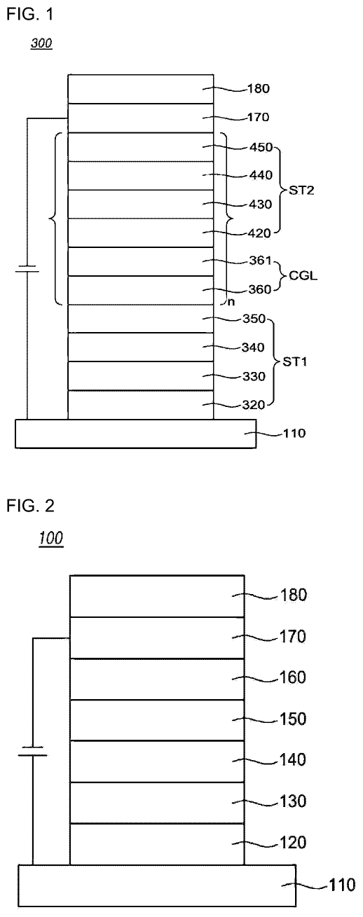

Referring to FIG. 2, the organic electronic element (100) according to the present invention comprises a first electrode (110), a second electrode (170), an organic material layer comprising a single compound or 2 or more compounds represented by Formula 32 between the first electrode (110) and the second electrode (170). In this case, the first electrode (110) may be an anode, the second electrode (170) may be a cathode, and in the case of an invert type, the first electrode may be a cathode and the second electrode may be an anode.

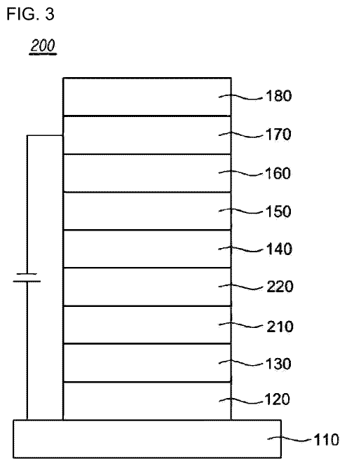

The organic material layer may sequentially comprise a hole injection layer (120), a hole transport layer (130), an emitting layer (140), an electron transport layer (150), and an electron injection layer (160) on the first electrode (110). In this case, the remaining layers except for the emitting layer (140) may not be formed. It may further include a hole blocking layer, an electron blocking layer, an emitting-auxiliary layer (220), a buffer layer (210), etc. and the electron transport layer (150) and the like may serve as a hole blocking layer. (See FIG. 3).

Also, the organic electronic element according to an embodiment of the present invention may further comprise a protective layer or a light efficiency enhancing layer (180). The light efficiency enhancing layer may be formed on one of both surfaces of the first electrode, the surface not in contact with the organic material layer or on one of both surfaces of the second electrode, the surface not in contact with the organic material layer.

The compound represented by Formula 32 according to the present invention applied to the organic material layer may be used as a material for the hole injection layer (120), the hole transport layer (130), the emitting-auxiliary layer (220), electron transport auxiliary layer, the electron transport layer (150), and an electron injection layer (160), a host or dopant of the emitting layer (140) or the light efficiency enhancing layer. Preferably, for example, the compound represented by Formulas 32 of the present invention may be used as a material for the hole transport layer.

Otherwise, even with the same core, the band gap, electrical characteristics, interface characteristics, etc. may vary depending on which position the substituent is bonded to, therefore the choice of core and the combination of sub-substituents bound thereto are also very important, and in particular, when the optimal combination of energy levels and T1 values and unique properties of materials (mobility, interfacial characteristics, etc.) of each organic material layer is achieved, a long lifespan and high efficiency can be achieved at the same time.

The present invention provides an organic electronic element comprising an anode; a cathode; and an organic layer between the anode and the cathode, wherein a compound represented by Formula 32 is comprised in the organic layer. Also, the organic layer comprises at least 2 or more stacks.

In another aspect, the present invention provides an organic electronic element comprising a charge generation region between stacks in 2 or more stacks in the organic layer.

Additionally, the charge generation region comprises an N-type charge generation layer and a P-type charge generation layer, and preferably, the P-type charge generation layer comprises a compound of Formula 32.

As another specific example, the present invention provides an organic electronic element characterized in that a compound of the same type or a different type of the compound represented by Formula 32 is mixed and used in the organic material layer.

Also, the present invention provides an electronic device comprising a display device including the organic electric element comprising the compound represented by Formula 32; and a control unit for driving the display device;

Hereinafter, Synthesis Examples of the compound represented by Formula (18) of the present invention and preparation examples of the organic electric element of the present invention will be described in detail by way of example, but are not limited to the following examples.

### Synthesis Example 1

Final products represented by Formula (18) according to the present invention can be prepared by reacting as follows, but are not limited thereto.

### Synthesis Example of 13-17

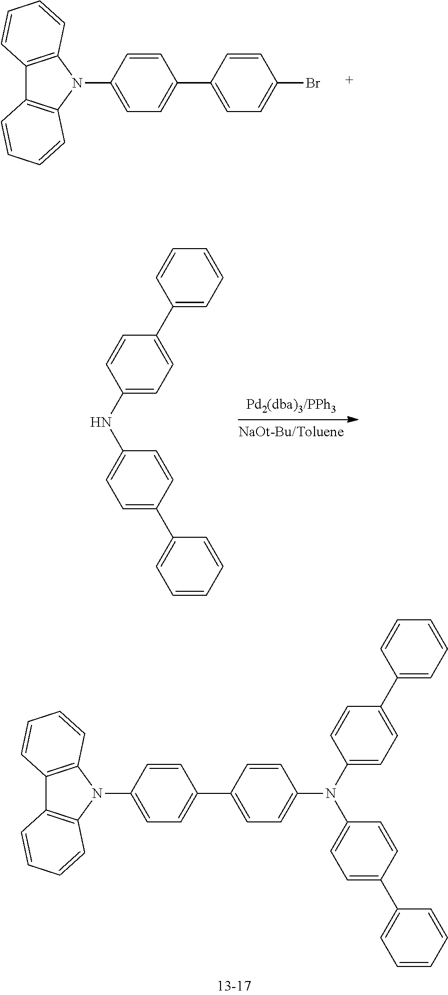 [SMILES: Brc1ccc(-c2ccc(-n3c4ccccc4c4ccccc43)cc2)cc1 ; c1ccc(-c2ccc(Nc3ccc(-c4ccccc4)cc3)cc2)cc1 ; c1ccc(-c2ccc(N(c3ccc(-c4ccccc4)cc3)c3ccc(-c4ccc(-n5c6ccccc6c6ccccc65)cc4)cc3)cc2)cc1]

9-(4′-bromo-[1,1′-biphenyl]-4-yl)-9H-carbazole (9.6 g, 24 mmol) was dissolved in toluene, and di([1,1′-biphenyl]-4-yl)amine (6.4 g, 20 mmol), Pd₂(dba)₃ (0.05 eq.), PPh₃ (0.1 eq.), NaOt-Bu (3 eq.) were added and refluxed with stirring at 100° C. at 24 hours. After the reaction was completed, the reaction mixture was extracted with ether and water. The organic layer was dried over MgSO₄ and concentrated. The resulting organic material was separated by silicagel column chromatography and recrystallization to obtain 12.9 g (yield: 84%) of the product.

### Synthesis Example of 13-32

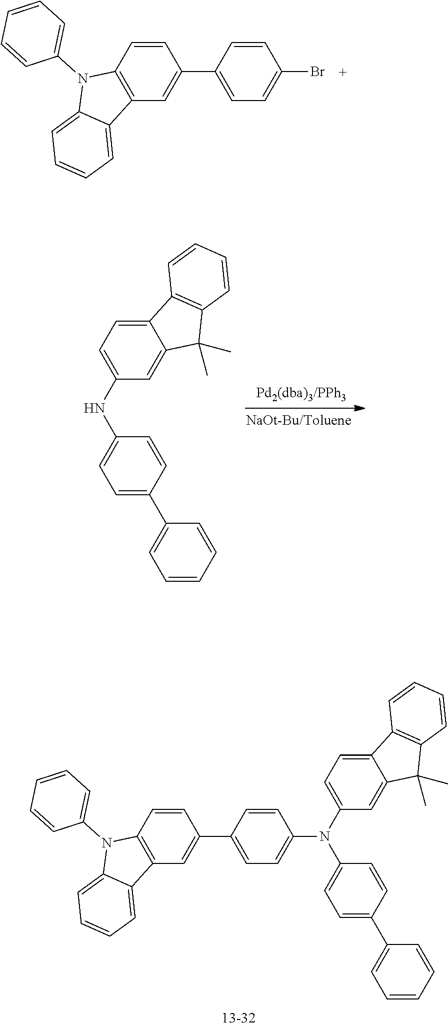 [SMILES: Brc1ccc(-c2ccc3c(c2)c2ccccc2n3-c2ccccc2)cc1 ; CC1(C)c2ccccc2-c2ccc(Nc3ccc(-c4ccccc4)cc3)cc21 ; CC1(C)c2ccccc2-c2ccc(N(c3ccc(-c4ccccc4)cc3)c3ccc(-c4ccc5c(c4)c4ccccc4n5-c4ccccc4)cc3)cc21]

3-(4-bromophenyl)-9-phenyl-9H-carbazole (9.6 g, 24 mmol) was dissolved in toluene, and N-([1,1′-biphenyl]-4-yl)-9,9-dimethyl-9H-fluoren-2-amine (7.2 g, 20 mmol), Pd₂(dba)₃ (0.05 eq.), PPh₃ (0.1 eq.), NaOt-Bu (3 eq.) were added and refluxed with stirring at 100° C. at 24 hours. After the reaction was completed, the reaction mixture was extracted with ether and water. The organic layer was dried over MgSO₄ and concentrated. The resulting organic material was separated by silicagel column chromatography and recrystallization to obtain 13.8 g (yield: 85%) of the product.

### Synthesis Example of 2-34

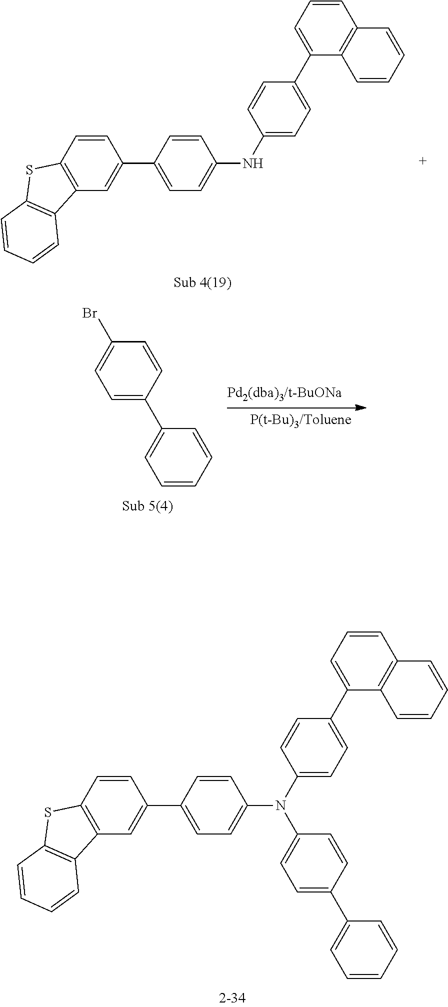 [SMILES: c1ccc2c(-c3ccc(Nc4ccc(-c5ccc6sc7ccccc7c6c5)cc4)cc3)cccc2c1 ; Brc1ccc(-c2ccccc2)cc1 ; c1ccc(-c2ccc(N(c3ccc(-c4ccc5sc6ccccc6c5c4)cc3)c3ccc(-c4cccc5ccccc45)cc3)cc2)cc1]

In a round bottom flask, Sub 4(19) (9.5 g, 20 mmol), Sub 5(4) (4.7 g, 20 mmol), Pd₂(dba)₃ (0.5 g, 0.6 mmol), P(t-Bu)₃ (0.2 g, 2 mmol), t-BuONa (5.8 g, 60 mmol), toluene (300 mL) were added and were carried out at 100° C. When the reaction was complete, the reaction mixture was extracted with CH₂Cl₂ and water. The organic layer was dried over MgSO₄ and concentrated. The resulting compound was separated by silicagel column chromatography and recrystallization to obtain 9.8 g (yield: 78%) of 2-34.

### Synthesis Example of 2-58

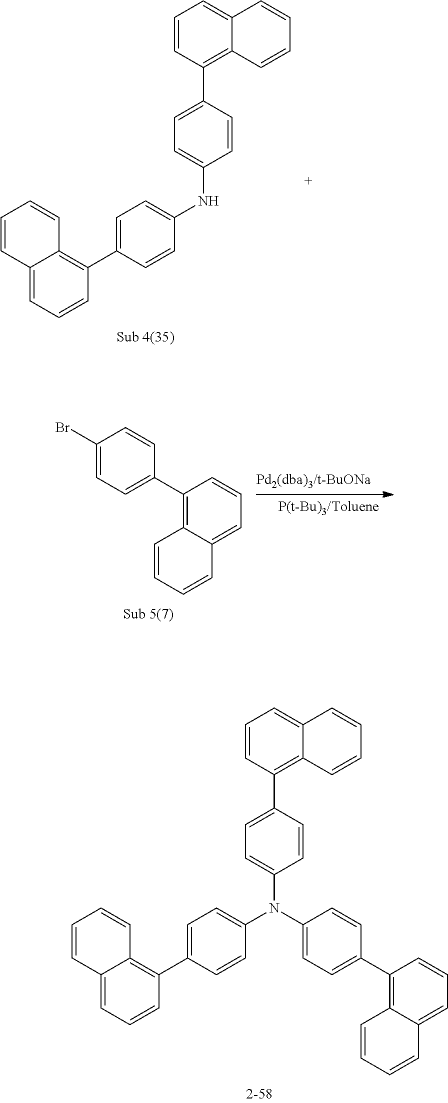 [SMILES: c1ccc2c(-c3ccc(Nc4ccc(-c5cccc6ccccc56)cc4)cc3)cccc2c1 ; Brc1ccc(-c2cccc3ccccc23)cc1 ; c1ccc2c(-c3ccc(N(c4ccc(-c5cccc6ccccc56)cc4)c4ccc(-c5cccc6ccccc56)cc4)cc3)cccc2c1]

In a round bottom flask, Sub 4(35) (8.4 g, 20 mmol), Sub 5(7) (5.7 g, 20 mmol), Pd₂(dba)₃ (0.5 g, 0.6 mmol), P(t-Bu)₃ (0.2 g, 2 mmol), t-BuONa (5.8 g, 60 mmol) and toluene (300 mL) were carried out in the same manner as in 2-34 to give 2-58. (10.4 g, 83%).

### Synthesis Example of 2-59

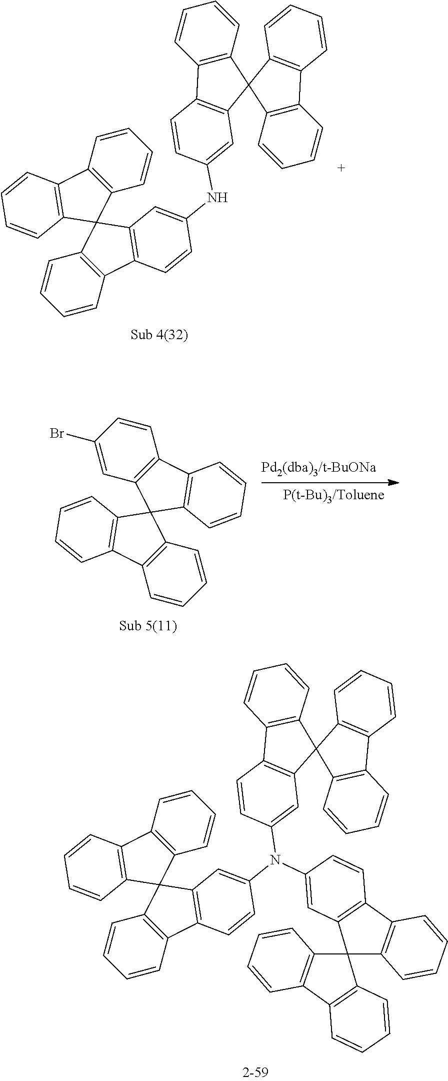 [SMILES: c1ccc2c(c1)-c1ccccc1C21c2ccccc2-c2ccc(Nc3ccc4c(c3)C3(c5ccccc5-c5ccccc53)c3ccccc3-4)cc21 ; Brc1ccc2c(c1)C1(c3ccccc3-c3ccccc31)c1ccccc1-2 ; c1ccc2c(c1)-c1ccccc1C21c2ccccc2-c2ccc(N(c3ccc4c(c3)C3(c5ccccc5-c5ccccc53)c3ccccc3-4)c3ccc4c(c3)C3(c5ccccc5-c5ccccc53)c3ccccc3-4)cc21]

In a round bottom flask, Sub 4(32) (12.9 g, 20 mmol), Sub 5(11) (7.9 g, 20 mmol), Pd₂(dba)₃ (0.5 g, 0.6 mmol), P(t-Bu)₃ (0.2 g, 2 mmol), t-BuONa (5.8 g, 60 mmol) and toluene (300 mL) were carried out in the same manner as in 2-34 to give 2-59. (5.2 g, 79%).

### Synthesis Example of 2-69

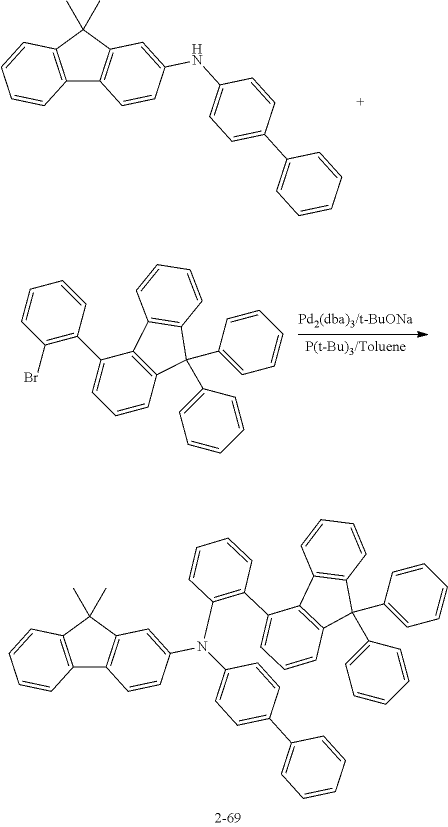 [SMILES: CC1(C)c2ccccc2-c2ccc(Nc3ccc(-c4ccccc4)cc3)cc21 ; Brc1ccccc1-c1cccc2c1-c1ccccc1C2(c1ccccc1)c1ccccc1 ; CC1(C)c2ccccc2-c2ccc(N(c3ccc(-c4ccccc4)cc3)c3ccccc3-c3cccc4c3-c3ccccc3C4(c3ccccc3)c3ccccc3)cc21]

In a round bottom flask, N-([1,1′-biphenyl]-4-yl)-9,9-dimethyl-9H-fluoren-2-amine (7.2 g, 20 mmol), 4-(2-bromophenyl)-9,9-diphenyl-9H-fluorene (9.5 g, 20 mmol), Pd₂(dba)₃ (0.5 g, 0.6 mmol), P(t-Bu)₃ (0.2 g, 2 mmol), t-BuONa (5.8 g, 60 mmol) and toluene (300 mL) were carried out in the same manner as in 2-34 to give 2-69. (12.2 g, 81%).

### Synthesis Example of 2-71

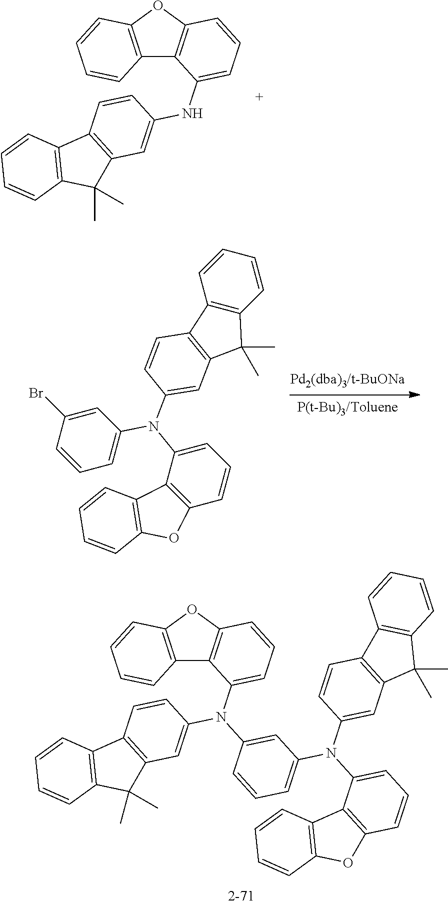 [SMILES: CC1(C)c2ccccc2-c2ccc(Nc3cccc4oc5ccccc5c34)cc21 ; CC1(C)c2ccccc2-c2ccc(N(c3cccc(Br)c3)c3cccc4oc5ccccc5c34)cc21 ; CC1(C)c2ccccc2-c2ccc(N(c3cccc(N(c4ccc5c(c4)C(C)(C)c4ccccc4-5)c4cccc5oc6ccccc6c45)c3)c3cccc4oc5ccccc5c34)cc21]

In a round bottom flask, N-(9,9-dimethyl-9H-fluoren-2-yl)dibenzo[b,d]furan-1-amine (7.5 g, 20 mmol), N-(3-bromophenyl)-N-(9,9-dimethyl-9H-fluoren-2-yl)dibenzo[b,d]furan-1-amine (10.6 g, 20 mmol), Pd₂(dba)₃ (0.5 g, 0.6 mmol), P(t-Bu)₃ (0.2 g, 2 mmol), t-BuONa (5.8 g, 60 mmol) and toluene (300 mL) were carried out in the same manner as in 2-34 to give 2-71. (12.9 g, 78%).

### Synthesis Example 2

Final products represented by Formula (30) according to the present invention can be prepared by reacting Sub 30 and Sub 31 as shown in Reaction Scheme 5 below, but are not limited thereto.

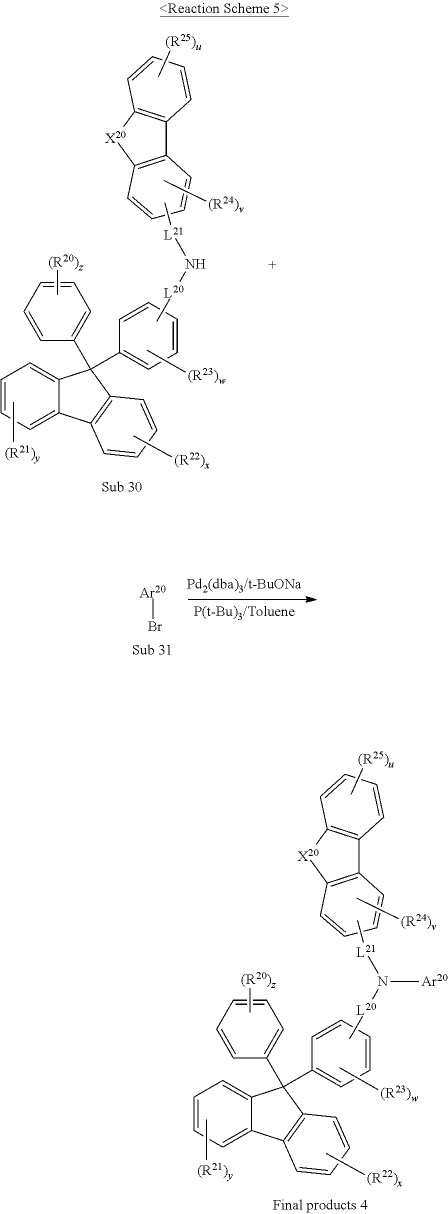

### Synthesis Example of Sub 30

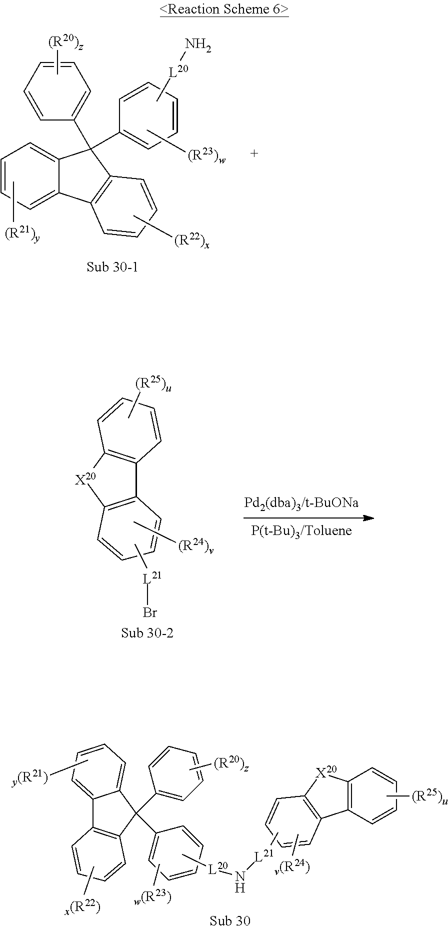

### Synthesis Example of Sub 30(81)

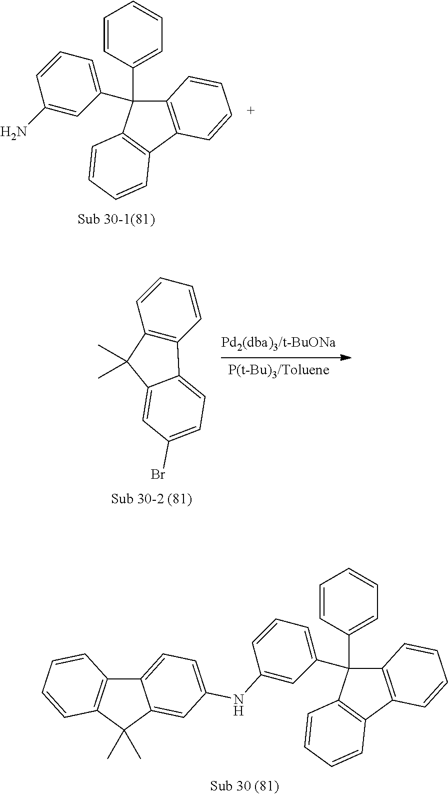 [SMILES: Nc1cccc(C2(c3ccccc3)c3ccccc3-c3ccccc32)c1 ; CC1(C)c2ccccc2-c2ccc(Br)cc21 ; CC1(C)c2ccccc2-c2ccc(Nc3cccc(C4(c5ccccc5)c5ccccc5-c5ccccc54)c3)cc21]

In a round bottom flask, 3-(9-phenyl-9H-fluoren-9-yl)aniline (6.7 g, 20 mmol), 2-bromo-9,9-dimethyl-9H-fluorene (5.5 g, 20 mmol), Pd₂(dba)₃ (0.5 g, 0.6 mmol), P(t-Bu)₃ (0.2 g, 2 mmol), t-BuONa (5.8 g, 60 mmol) and toluene (300 mL) were carried out in the same manner as in 2-34 to give Sub 30(81). (8.83 g, 84%).

### Examples of Sub 31

Examples of Sub 31 are as follows, but are not limited thereto.

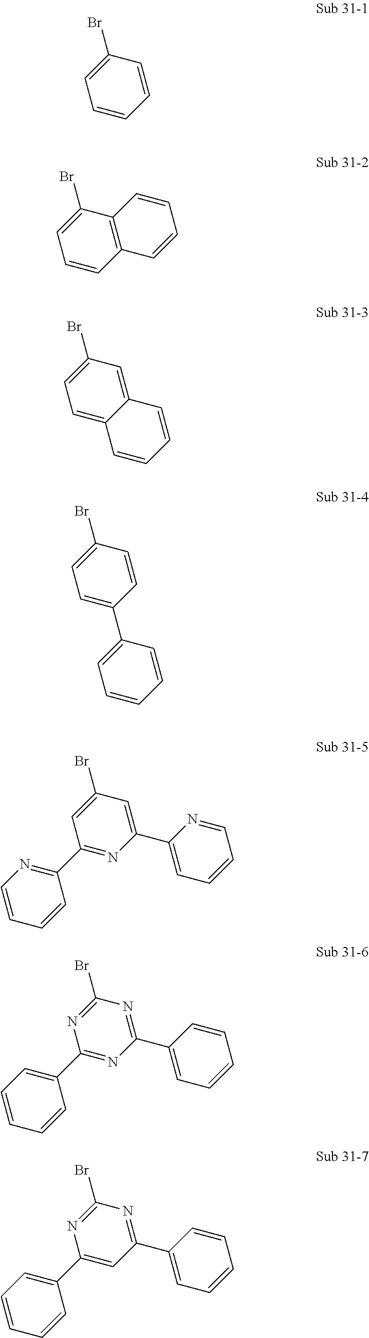

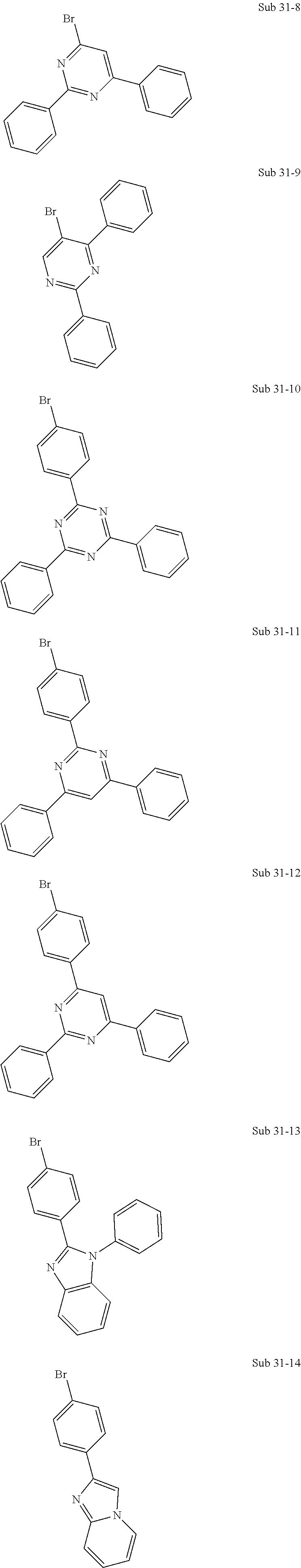

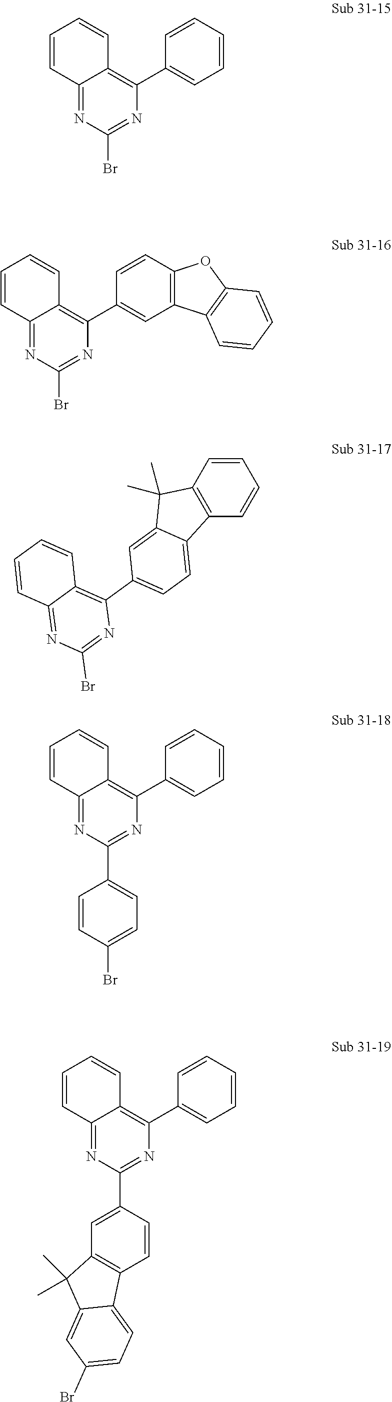

TABLE 5

| compound | FD-MS | compound | FD-MS |
|---|---|---|---|
| Sub 31-1 | m/z = 155.96(C₆H₅Br = 157.01) | Sub 31-2 | m/z = 205.97(C₁₀H₇Br = 207.07) |
| Sub 31-3 | m/z = 205.97(C₁₀H₇Br = 207.07) | Sub 31-4 | m/z = 231.99(C₁₂H₉Br = 233.10) |
| Sub 31-5 | m/z = 309.02(C₁₇H₁₂BrN = 310.19) | Sub 31-6 | m/z = 311.01(C₁₅H₁₀BrN₃ = 312.16) |
| Sub 31-7 | m/z = 310.01(C₁₆H₁₁BrN₂ = 311.18) | Sub 31-8 | m/z = 310.01(C₁₆H₁₁BrN₂ = 311.18) |
| Sub 31-9 | m/z = 310.01(C₁₆H₁₁BrN₂ = 311.18) | Sub 31-10 | m/z = 387.04(C₂₁H₁₄BrN₃ = 388.26) |
| Sub 31-11 | m/z = 386.04(C₂₂H₁₅BrN₂ = 387.27) | Sub 31-12 | m/z = 386.04(C₂₂H₁₅BrN₂ = 387.27) |
| Sub 31-13 | m/z = 348.03(C₁₉H₁₃BrN₂ = 349.22) | Sub 31-14 | m/z = 271.99(C₁₃H₉BrN₂ = 273.13) |
| Sub 31-15 | m/z = 283.99(C₁₄H₉BrN₂ = 285.14) | Sub 31-16 | m/z = 374.01(C₂₀H₁₁BrN₂O = 375.22) |
| Sub 31-17 | m/z = 400.06(C₂₃H₁₇BrN₂ = 401.30) | Sub 31-18 | m/z = ₃₆0.03(C₂₀H₁₃BrN₂ = 361.23) |
| Sub 31-19 | m/z = 476.09(C₂₉H₂₁BrN₂ = 477.₃₉) | | |

### Synthesis Example of 2-72

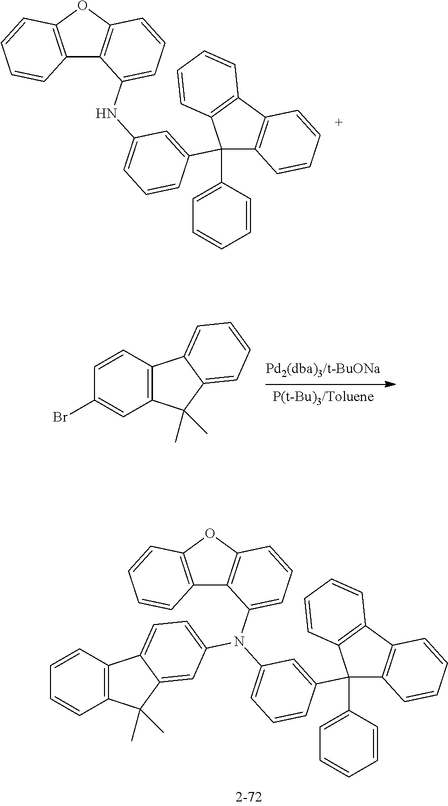 [SMILES: c1ccc(C2(c3cccc(Nc4cccc5oc6ccccc6c45)c3)c3ccccc3-c3ccccc32)cc1 ; CC1(C)c2ccccc2-c2ccc(Br)cc21 ; CC1(C)c2ccccc2-c2ccc(N(c3cccc(C4(c5ccccc5)c5ccccc5-c5ccccc54)c3)c3cccc4oc5ccccc5c34)cc21]

In a round bottom flask, N-(3-(9-phenyl-9H-fluoren-9-yl)phenyl)dibenzo[b,d]furan-1-amine (10.0 g, 20 mmol), 2-bromo-9,9-dimethyl-9H-fluorene (5.5 g, 20 mmol), Pd₂(dba)₃ (0.5 g, 0.6 mmol), P(t-Bu)₃ (0.2 g, 2 mmol), t-BuONa (5.8 g, 60 mmol) and toluene (300 mL) were carried out in the same manner as in 2-34 to give 2-72. (11.1 g, 80%).

### Synthesis Example of 2-81

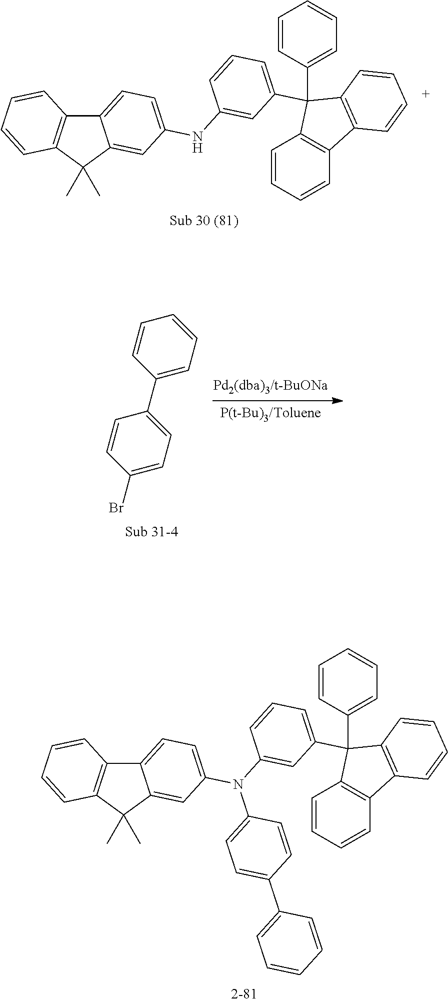 [SMILES: CC1(C)c2ccccc2-c2ccc(Nc3cccc(C4(c5ccccc5)c5ccccc5-c5ccccc54)c3)cc21 ; Brc1ccc(-c2ccccc2)cc1 ; CC1(C)c2ccccc2-c2ccc(N(c3ccc(-c4ccccc4)cc3)c3cccc(C4(c5ccccc5)c5ccccc5-c5ccccc54)c3)cc21]

In a round bottom flask, Sub 30(81) (10.3 g, 20 mmol), Sub 31-4 (4.66 g, 20 mmol), Pd₂(dba)₃ (0.5 g, 0.6 mmol), P(t-Bu)₃ (0.2 g, 2 mmol), t-BuONa (5.8 g, 60 mmol) and toluene (300 mL) were carried out in the same manner as in 2-34 to give 2-81. (10.3 g, 76%).

### Synthesis Example of 2-161

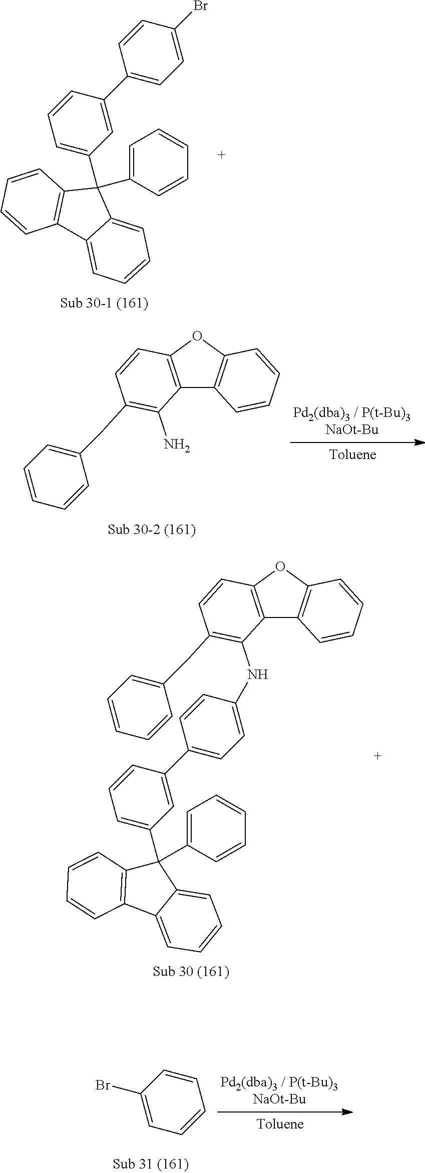 [SMILES: Brc1ccc(-c2cccc(C3(c4ccccc4)c4ccccc4-c4ccccc43)c2)cc1 ; Nc1c(Cc2ccccc2)ccc2oc3ccccc3c12 ; c1ccc(Cc2ccc3oc4ccccc4c3c2Nc2ccc(-c3cccc(C4(c5ccccc5)c5ccccc5-c5ccccc54)c3)cc2)cc1 ; Brc1ccccc1]

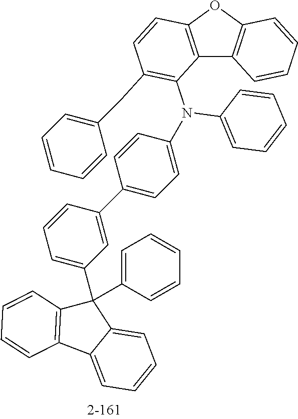 [SMILES: c1ccc(Cc2ccc3oc4ccccc4c3c2N(c2ccccc2)c2ccc(-c3cccc(C4(c5ccccc5)c5ccccc5-c5ccccc54)c3)cc2)cc1]

### 1) Sub 30 (161)

In a round bottom flask, Sub 30-1 (161) (20.0 g, 42.2 mmol) and Sub 30-2 (161) (11.0 g, 42.2 mmol), Pd₂(dba)₃ (1.2 g, 1.3 mmol), P(t-Bu)₃ (0.5 g, 2.5 mmol), NaOt-Bu (8.1 g, 84.5 mmol), toluene (211 mL) were carried out in the same manner as in 2-3 to give Sub 30 (161). (19.7 g, 71.6%).

2) 2-161

In a round bottom flask, Sub 30 (161) (10.0 g, 15.3 mmol) and Sub 31 (161) (2.4 g, 15.3 mmol), Pd₂(dba)₃ (0.4 g, 0.5 mmol), P(t-Bu)₃ (0.2 g, 0.9 mmol), NaOt-Bu (2.9 g, 30.6 mmol), toluene (76 mL) were carried out in the same manner as in 2-3 to give 2-161. (7.9 g, 70.9%).

### Synthesis Example of 2-166

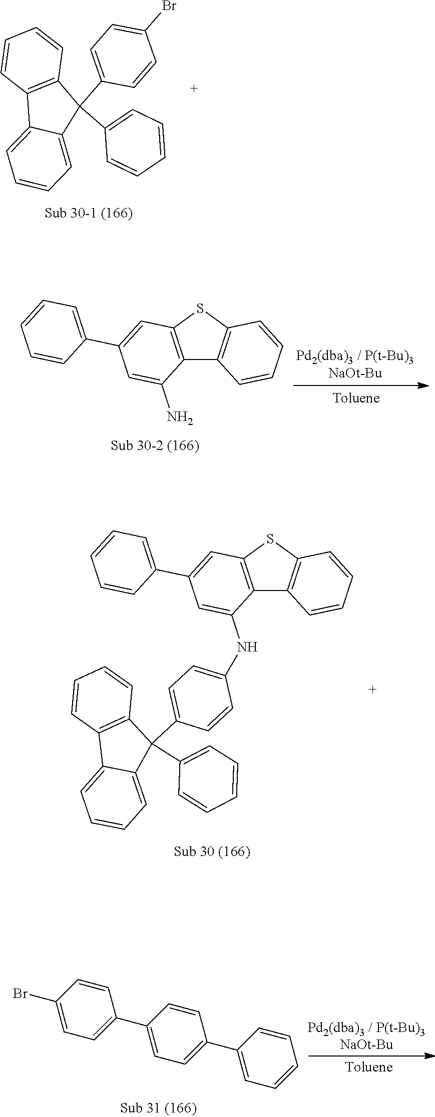 [SMILES: Brc1ccc(C2(c3ccccc3)c3ccccc3-c3ccccc32)cc1 ; Nc1cc(-c2ccccc2)cc2sc3ccccc3c12 ; c1ccc(-c2cc(Nc3ccc(C4(c5ccccc5)c5ccccc5-c5ccccc54)cc3)c3c(c2)sc2ccccc23)cc1 ; Brc1ccc(-c2ccc(-c3ccccc3)cc2)cc1]

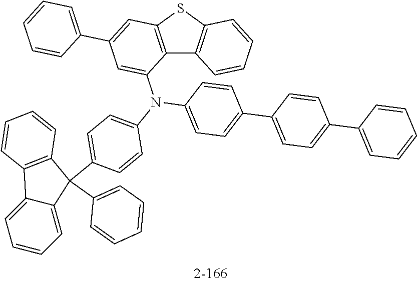 [SMILES: c1ccc(-c2ccc(-c3ccc(N(c4ccc(C5(c6ccccc6)c6ccccc6-c6ccccc65)cc4)c4cc(-c5ccccc5)cc5sc6ccccc6c45)cc3)cc2)cc1]

### 1) Sub 30 (166)

In a round bottom flask, Sub 30-1 (166) (20.0 g, 50.3 mmol) and Sub 30-2 (166) (13.9 g, 50.3 mmol), Pd₂(dba)₃ (1.4 g, 1.5 mmol), P(t-Bu)₃ (0.6 g, 3.0 mmol), NaOt-Bu (9.7 g, 100.7 mmol), toluene (252 mL) were carried out in the same manner as in 2-3 to give Sub 30 (166). (21.0 g, 70.5%).

2) 2-166

In a round bottom flask, Sub 30 (166) (10.0 g, 16.9 mmol) and Sub 31 (166) (5.2 g, 16.9 mmol), Pd₂(dba)₃ (0.5 g, 0.5 mmol), P(t-Bu)₃ (0.2 g, 1.0 mmol), NaOt-Bu (3.2 g, 33.8 mmol), toluene (84 mL) were carried out in the same manner as in 2-3 to give 2-166. (9.8 g, 71.0%).

### Synthesis Example of 2-167

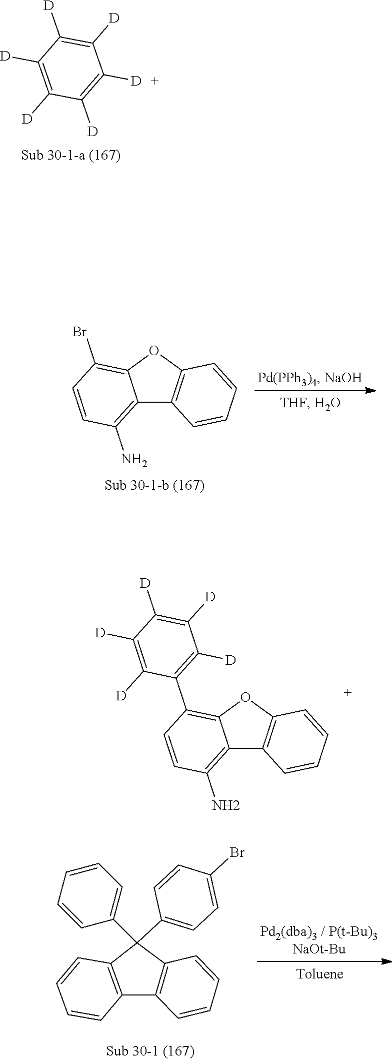 [SMILES: [2H]c1c([2H])c([2H])c([2H])c([2H])c1[2H] ; Nc1ccc(Br)c2oc3ccccc3c12 ; [2H]c1c([2H])c([2H])c(-c2ccc(N)c3c2oc2ccccc23)c([2H])c1[2H] ; Brc1ccc(C2(c3ccccc3)c3ccccc3-c3ccccc32)cc1]

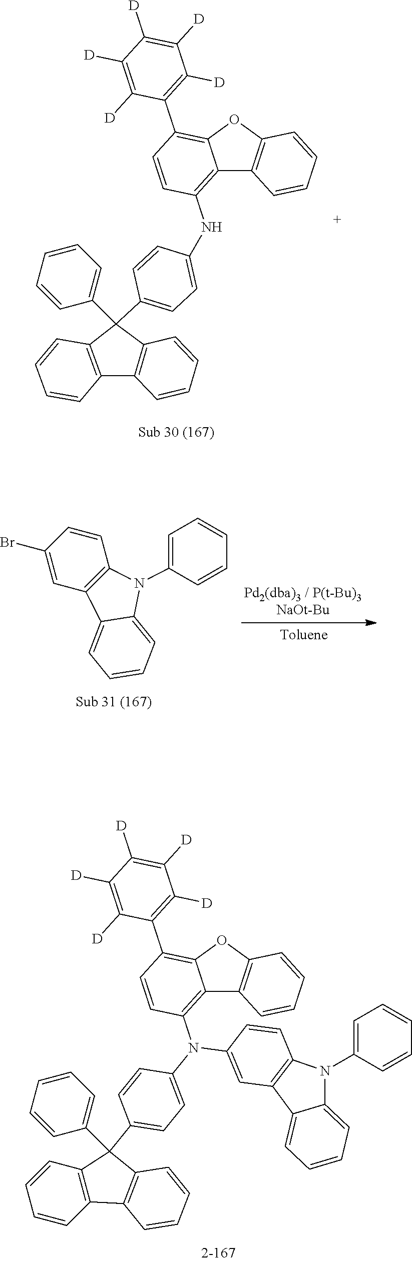 [SMILES: [2H]c1c([2H])c([2H])c(-c2ccc(Nc3ccc(C4(c5ccccc5)c5ccccc5-c5ccccc54)cc3)c3c2oc2ccccc23)c([2H])c1[2H] ; Brc1ccc2c(c1)c1ccccc1n2-c1ccccc1 ; [2H]c1c([2H])c([2H])c(-c2ccc(N(c3ccc(C4(c5ccccc5)c5ccccc5-c5ccccc54)cc3)c3ccc4c(c3)c3ccccc3n4-c3ccccc3)c3c2oc2ccccc23)c([2H])c1[2H]]

### 1) Sub 30-2 (167)

In a round bottom flask, Sub 30-1-a (167) (20.0 g, 157.5 mmol) and Sub 30-1-b (167) (41.3 g, 157.5 mmol), Pd(PPh₃)₄ (10.9 g, 9.5 mmol), K₂CO₃ (65.3 g, 472.6 mmol), THF (788 ml), water (394 ml) were carried out in the same manner as in 2-3 to give Sub 30-2 (167). (33.8 g, 81.2%).

### 2) Sub 30 (167)

In a round bottom flask, Sub 30-1 (167) (20.0 g, 50.3 mmol) and Sub 30-2 (167) (13.3 g, 50.3 mmol), Pd₂(dba)₃ (1.4 g, 1.5 mmol), P(t-Bu)₃ (0.6 g, 3.0 mmol), NaOt-Bu (9.7 g, 100.7 mmol), toluene (252 mL) were carried out in the same manner as in 2-3 to give Sub 30 (167). (20.7 g, 70.7%).

3) 2-167

In a round bottom flask, Sub 30 (167) (10.0 g, 17.2 mmol), Sub 31 (167) (5.5 g, 17.2 mmol), Pd₂(dba)₃ (0.5 g, 0.5 mmol), P(t-Bu)₃ (0.2 g, 1.0 mmol), NaOt-Bu (3.3 g, 34.4 mmol), toluene (86 mL) were carried out in the same manner as in 2-3 to give 2-167. (10.1 g, 71.3%).

### Synthesis Example of 2-180

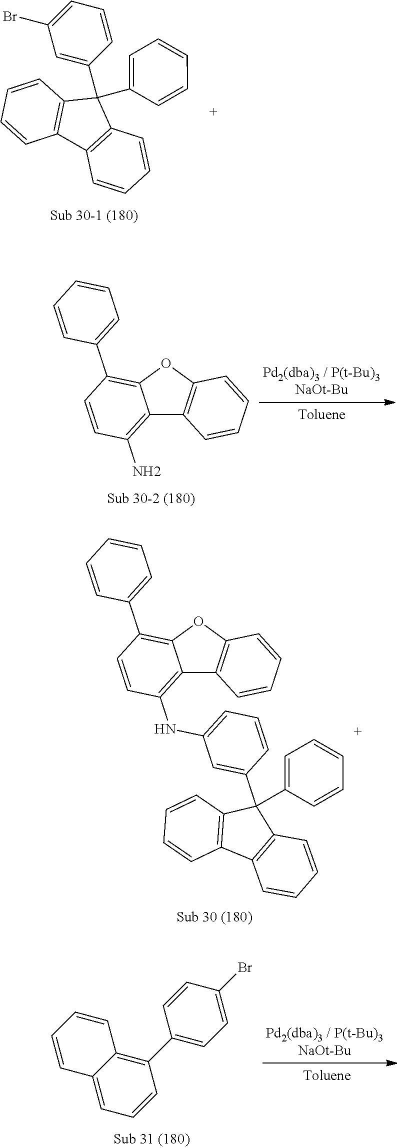 [SMILES: Brc1cccc(C2(c3ccccc3)c3ccccc3-c3ccccc32)c1 ; Nc1ccc(-c2ccccc2)c2oc3ccccc3c12 ; c1ccc(-c2ccc(Nc3cccc(C4(c5ccccc5)c5ccccc5-c5ccccc54)c3)c3c2oc2ccccc23)cc1 ; Brc1ccc(-c2cccc3ccccc23)cc1]

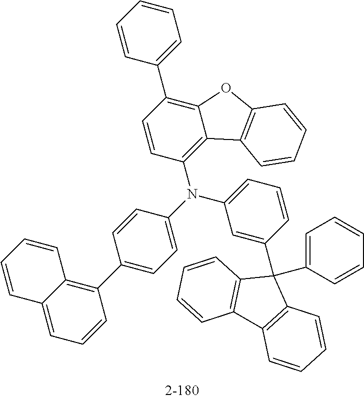 [SMILES: c1ccc(-c2ccc(N(c3ccc(-c4cccc5ccccc45)cc3)c3cccc(C4(c5ccccc5)c5ccccc5-c5ccccc54)c3)c3c2oc2ccccc23)cc1]

### 1) Sub 30 (180)

In a round bottom flask, Sub 30-1 (180) (20.0 g, 50.3 mmol) and Sub 30-2 (180) (13.1 g, 50.3 mmol), Pd₂(dba)₃ (1.4 g, 1.5 mmol), P(t-Bu)₃ (0.6 g, 3.0 mmol), NaOt-Bu (9.7 g, 100.7 mmol), toluene (252 mL) were carried out in the same manner as in 2-3 to give Sub 30 (180). (20.7 g, 71.3%).

2) 2-180

In a round bottom flask, Sub 30 (180) (10.0 g, 17.4 mmol) and Sub 31 (180) (4.9 g, 17.4 mmol), Pd₂(dba)₃ (0.5 g, 0.5 mmol), P(t-Bu)₃ (0.2 g, 1.0 mmol), NaOt-Bu (3.3 g, 34.7 mmol), toluene (87 mL) were carried out in the same manner as in 2-3 to give 2-180. (9.8 g, 72.4%).

### Synthesis Example of 2-206

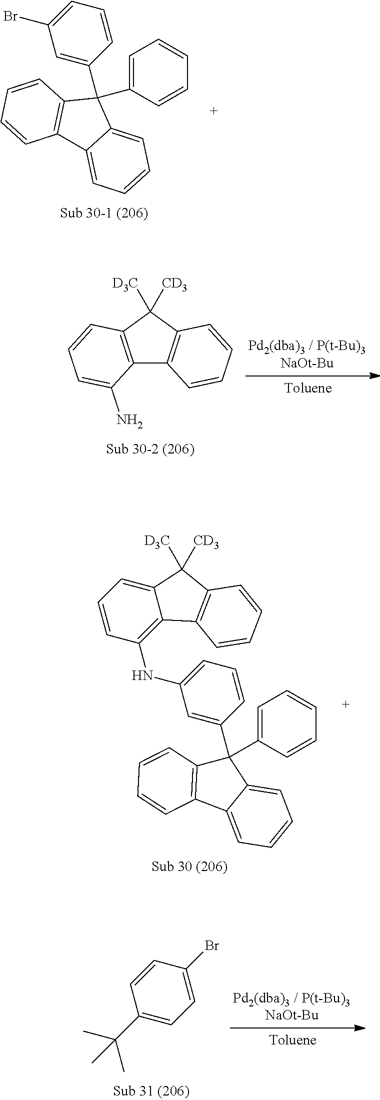 [SMILES: Brc1cccc(C2(c3ccccc3)c3ccccc3-c3ccccc32)c1 ; [2H]C([2H])([2H])C1(C([2H])([2H])[2H])c2ccccc2-c2c(N)cccc21 ; [2H]C([2H])([2H])C1(C([2H])([2H])[2H])c2ccccc2-c2c(Nc3cccc(C4(c5ccccc5)c5ccccc5-c5ccccc54)c3)cccc21 ; CC(C)(C)c1ccc(Br)cc1]

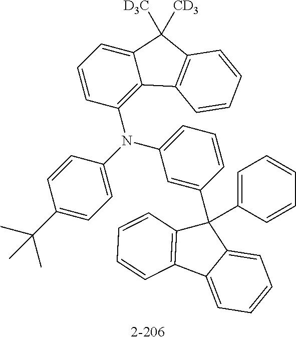 [SMILES: [2H]C([2H])([2H])C1(C([2H])([2H])[2H])c2ccccc2-c2c(N(c3ccc(C(C)(C)C)cc3)c3cccc(C4(c5ccccc5)c5ccccc5-c5ccccc54)c3)cccc21]

### 1) Sub 30 (206)

In a round bottom flask, Sub 30-1 (206) (20.0 g, 50.3 mmol), Sub 30-2 (206) (10.8 g, 50.3 mmol), Pd₂(dba)₃ (1.4 g, 1.5 mmol), P(t-Bu)₃ (0.6 g, 2.0 mmol), NaOt-Bu (9.7 g, 100.7 mmol), toluene (252 mL) were carried out in the same manner as in 2-3 to give Sub 30 (206). (19.0 g, 71.1%).

2) 2-206

In a round bottom flask, Sub 30 (206) (10.0 g, 18.8 mmol), Sub 31 (206) (4.3 g, 18.8 mmol), Pd₂(dba)₃ (0.5 g, 0.6 mmol), P(t-Bu)₃ (0.2 g, 1.1 mmol), NaOt-Bu (3.6 g, 37.6 mmol), toluene (94 mL) were carried out in the same manner as in 2-3 to give 2-206. (8.7 g, 69.7%).

### Synthesis Example of 2-212

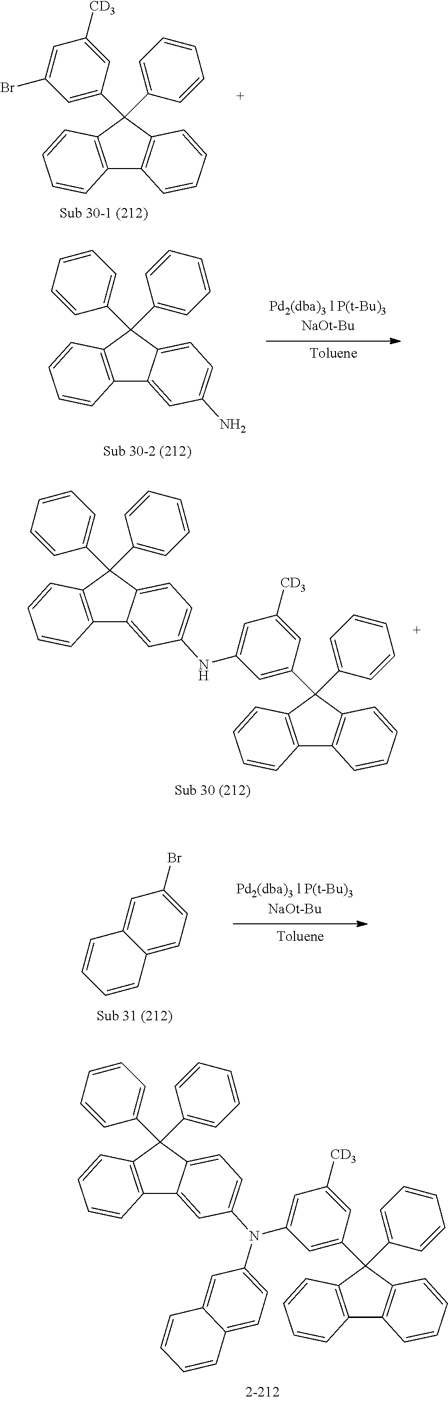 [SMILES: [2H]C([2H])([2H])c1cc(Br)cc(C2(c3ccccc3)c3ccccc3-c3ccccc32)c1 ; Nc1ccc2c(c1)-c1ccccc1C2(c1ccccc1)c1ccccc1 ; [2H]C([2H])([2H])c1cc(Nc2ccc3c(c2)-c2ccccc2C3(c2ccccc2)c2ccccc2)cc(C2(c3ccccc3)c3ccccc3-c3ccccc32)c1 ; Brc1ccc2ccccc2c1 ; [2H]C([2H])([2H])c1cc(N(c2ccc3c(c2)-c2ccccc2C3(c2ccccc2)c2ccccc2)c2ccc3ccccc3c2)cc(C2(c3ccccc3)c3ccccc3-c3ccccc32)c1]

### 1) Sub 30 (212)

In a round bottom flask, Sub 30-1 (212) (20.0 g, 48.3 mmol), Sub 30-2 (212) (16.1 g, 48.3 mmol), Pd₂(dba)₃ (1.3 g, 1.5 mmol), P(t-Bu)₃ (0.6 g, 2.9 mmol), NaOt-Bu (9.3 g, 96.5 mmol), toluene (241 mL) were carried out in the same manner as in 2-3 to give Sub 30 (212). (23.2 g, 72.0%).

2) 2-212

In a round bottom flask, Sub 30 (212) (10.0 g, 15.0 mmol), Sub 31 (212) (3.1 g, 15.0 mmol), Pd₂(dba)₃ (0.4 g, 0.5 mmol), P(t-Bu)₃ (0.2 g, 0.9 mmol), NaOt-Bu (2.9 g, 30.0 mmol), toluene (75 mL) were carried out in the same manner as in 2-3 to give 2-212. (8.3 g, 70.2%).

### Synthesis Example of 2-217

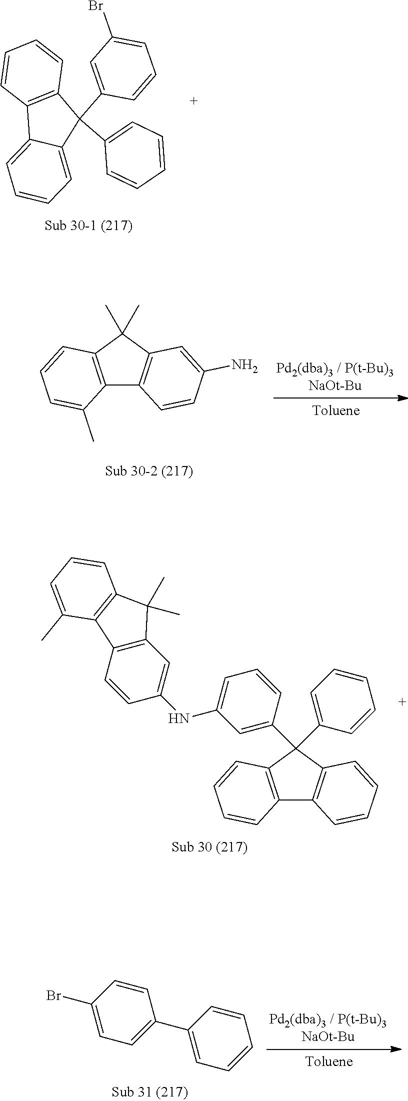 [SMILES: Brc1cccc(C2(c3ccccc3)c3ccccc3-c3ccccc32)c1 ; Cc1cccc2c1-c1ccc(N)cc1C2(C)C ; Cc1cccc2c1-c1ccc(Nc3cccc(C4(c5ccccc5)c5ccccc5-c5ccccc54)c3)cc1C2(C)C ; Brc1ccc(-c2ccccc2)cc1]

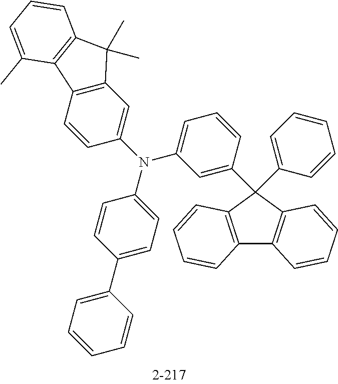 [SMILES: Cc1cccc2c1-c1ccc(N(c3ccc(-c4ccccc4)cc3)c3cccc(C4(c5ccccc5)c5ccccc5-c5ccccc54)c3)cc1C2(C)C]

### 1) Sub 30 (217)

In a round bottom flask, Sub 30-1 (217) (20.0 g, 50.3 mmol), Sub 30-2 (217) (11.2 g, 50.3 mmol), Pd₂(dba)₃ (1.4 g, 1.5 mmol), P(t-Bu)₃ (0.6 g, 2.0 mmol), NaOt-Bu (9.7 g, 100.7 mmol), toluene (252 mL) were carried out in the same manner as in 2-3 to give Sub 30 (217). (21.4 g, 71.5%).

2) 2-217

In a round bottom flask, Sub 30 (217) (10.0 g, 18.5 mmol), Sub 31 (217) (4.3 g, 18.5 mmol), Pd₂(dba)₃ (0.5 g, 0.6 mmol), P(t-Bu)₃ (0.2 g, 1.1 mmol), NaOt-Bu (3.6 g, 37.1 mmol), toluene (93 mL) were carried out in the same manner as in 2-3 to give 2-217. (9.1 g, 70.7%).

### Synthesis Example of 2-222

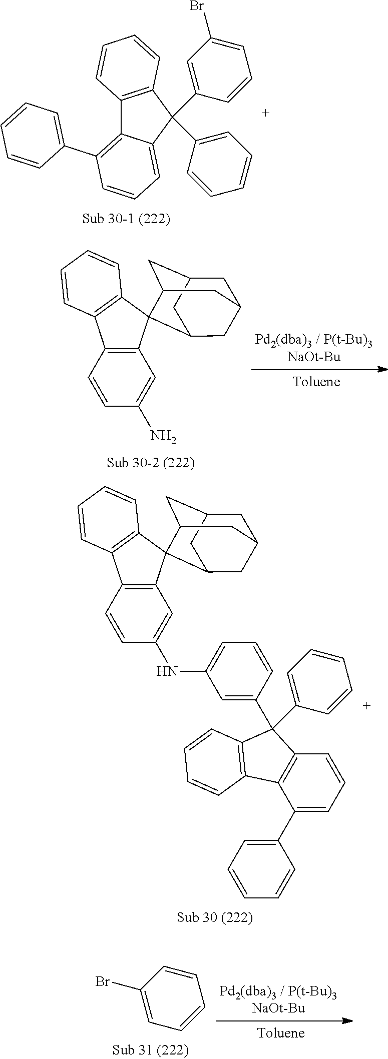 [SMILES: Brc1cccc(C2(c3ccccc3)c3ccccc3-c3c(-c4ccccc4)cccc32)c1 ; Nc1ccc2c(c1)C1(c3ccccc3-2)C2CC3CC(C2)CC1C3 ; c1ccc(-c2cccc3c2-c2ccccc2C3(c2ccccc2)c2cccc(Nc3ccc4c(c3)C3(c5ccccc5-4)C4CC5CC(C4)CC3C5)c2)cc1 ; Brc1ccccc1]

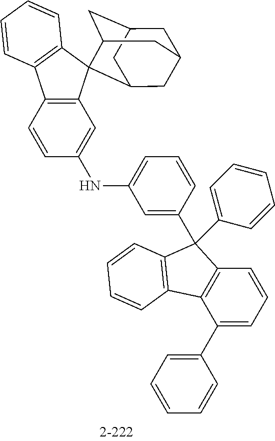 [SMILES: c1ccc(-c2cccc3c2-c2ccccc2C3(c2ccccc2)c2cccc(Nc3ccc4c(c3)C3(c5ccccc5-4)C4CC5CC(C4)CC3C5)c2)cc1]

### 1) Sub 30 (222)

In a round bottom flask, Sub 30-1 (222) (20.0 g, 42.2 mmol), Sub 30-2 (222) (12.7 g, 42.2 mmol), Pd₂(dba)₃ (1.2 g, 1.3 mmol), P(t-Bu)₃ (0.5 g, 2.5 mmol), NaOt-Bu (8.1 g, 84.5 mmol), toluene (211 mL) were carried out in the same manner as in 2-3 to give Sub 30 (222). (20.9 g, 71.2%).

2) 2-222

In a round bottom flask, Sub 30 (222) (10.0 g, 14.4 mmol), Sub 31 (222) (2.3 g, 14.4 mmol), Pd₂(dba)₃ (0.4 g, 0.4 mmol), P(t-Bu)₃ (0.2 g, 0.9 mmol), NaOt-Bu (2.8 g, 28.8 mmol), toluene (72 mL) were carried out in the same manner as in 2-3 to give 2-222. (7.9 g, 71.4%).

### Synthesis Example of 2-233

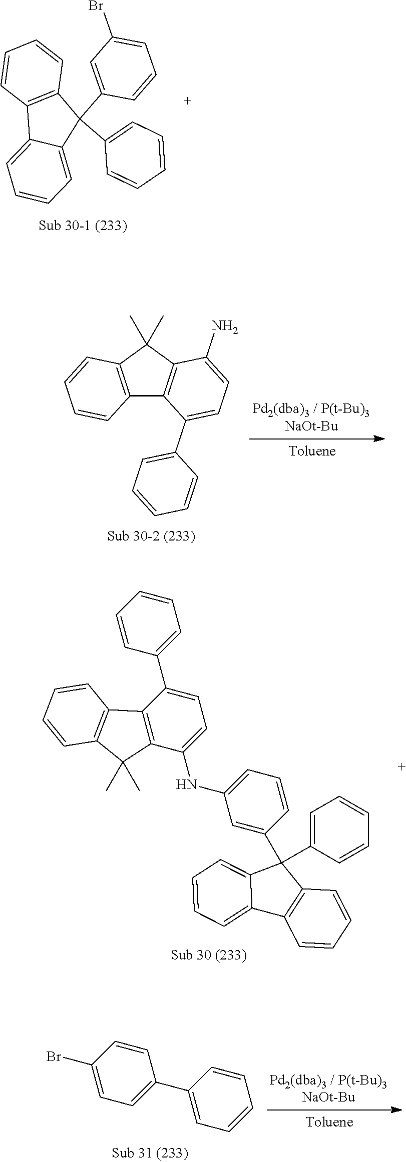 [SMILES: Brc1cccc(C2(c3ccccc3)c3ccccc3-c3ccccc32)c1 ; CC1(C)c2ccccc2-c2c(-c3ccccc3)ccc(N)c21 ; CC1(C)c2ccccc2-c2c(-c3ccccc3)ccc(Nc3cccc(C4(c5ccccc5)c5ccccc5-c5ccccc54)c3)c21 ; Brc1ccc(-c2ccccc2)cc1]

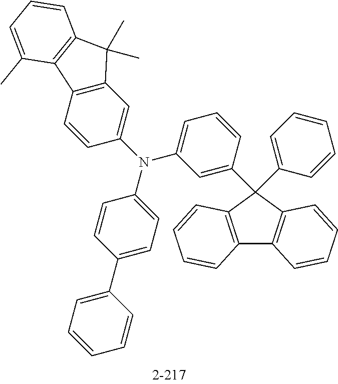 [SMILES: Cc1cccc2c1-c1ccc(N(c3ccc(-c4ccccc4)cc3)c3cccc(C4(c5ccccc5)c5ccccc5-c5ccccc54)c3)cc1C2(C)C]

### 1) Sub 30 (233)

In a round bottom flask, Sub 30-1 (233) (20.0 g, 50.3 mmol), Sub 30-2 (233) (14.4 g, 50.3 mmol), Pd₂(dba)₃ (1.4 g, 1.5 mmol), P(t-Bu)₃ (0.6 g, 2.0 mmol), NaOt-Bu (9.7 g, 100.7 mmol), toluene (252 mL) were carried out in the same manner as in 2-3 to give Sub 30 (233). (21.5 g, 70.9%).

2) 2-233

In a round bottom flask, Sub 30 (233) (10.0 g, 16.6 mmol), Sub 31 (233) (4.3 g, 16.6 mmol), Pd₂(dba)₃ (0.5 g, 0.5 mmol), P(t-Bu)₃ (0.2 g, 1.0 mmol), NaOt-Bu (3.2 g, 33.2 mmol), toluene (83 mL) were carried out in the same manner as in 2-3 to give 2-233. (8.9 g, 70.8%).

TABLE 6

| compound | FD-MS | compound | FD-MS |
|---|---|---|---|
| 2-81 | m/z = 677.31(C₅₂H₃₉N = 677.89) | 2-82 | m/z = 651.26(C₄₉H₃₃NO = 651.81) |
| 2-83 | m/z = 641.22(C₄₇H₃₁NS = 641.83) | 2-84 | m/z = 700.29(C₅₃H₃₆N₂ = 700.89) |
| 2-85 | m/z = 677.31(C₅₂H₃₉N = 677.89) | 2-86 | m/z = 651.26(C₄₉H₃₃NO = 651.81) |
| 2-87 | m/z = 843.30(C₆₃H₄₁NS = 844.09) | 2-88 | m/z = 701.28(C₅₂H₃₅N₃ = 701.87) |
| 2-89 | m/z = 677.31(C₅₂H₃₉N = 677.89) | 2-90 | m/z = 729.28(C₅₃H₃₅N₃O = 729.88) |
| 2-91 | m/z = 912.29(C₆₄H₄₀N₄OS = 913.11) | 2-92 | m/z = 878.34(C₆₅H₄₂N₄ = 879.08) |
| 2-93 | m/z = 805.35(C₆₀H₄₃N₃ = 806.03) | 2-94 | m/z = 906.34(C₆₆H₄₂N₄O = 907.09) |
| 2-95 | m/z = 769.26(C₅₅H₃₅N₃S = 769.97) | 2-96 | m/z = 884.30(C₆₃H₄₀N₄S = 885.10) |
| 2-97 | m/z = 767.36(C₅₉H₄₅N = 768.02) | 2-98 | m/z = 815.32(C₆₂H₄₁NO = 816.02) |
| 2-99 | m/z = 829.28(C₆₂H₃₉NS = 830.06) | 2-100 | m/z = 781.35(C₅₉H₃₅D₅N₂ = 782.01) |
| 2-101 | m/z = 695.30(C₅₂H₃₈FN = 695.88) | 2-102 | m/z = 753.34(C₅₈H₄₃N = 753.99) |
| 2-103 | m/z = 803.36(C₆₂H₄₅N = 804.05) | 2-104 | m/z = 6829.37(C₆₄H₄₇N = 830.09) |
| 2-105 | m/z = 918.40(C₇₀H₅₀N₂ = 919.18) | 2-106 | m/z = 601.28(C₄₆H₃₅N = 601.79) |
| 2-107 | m/z = 677.31(C₅₂H₃₉N = 677.89) | 2-108 | m/z = 753.34(C₅₈H₄₃N = 753.99) |
| 2-109 | m/z = 701.31(C₅₄H₃₉N = 701.91) | 2-110 | m/z = 677.31(C₅₂H₃₉N = 677.89) |
| 2-111 | m/z = 677.31(C₅₂H₃₉N = 677.89) | 2-112 | m/z = 682.34(C₅₂H₃₄D₅N = 682.92) |
| 2-113 | m/z = 701.31(C₅₄H₃₉N = 701.91) | 2-114 | m/z = 619.27(C₄₆H₃₄FN = 619.78) |
| 2-115 | m/z = 753.34(C₅₈H₄₃N = 753.99) | 2-116 | m/z = 803.36(C₆₂H₄₅N = 804.05) |
| 2-117 | m/z = 701.31(C₅₄H₃₉N = 701.91) | 2-118 | m/z = 651.29(C₅₀H₃₇N = 651.85) |
| 2-119 | m/z = 727.32(C₅₆H₄₁N = 727.95) | 2-120 | m/z = 758.37(C₅₈H₃₈D₅N = 759.02) |
| 2-121 | m/z = 757.37(C₅₈H₄₇N = 758.02) | 2-122 | m/z = 753.34(C₅₈H₄₃N = 753.99) |
| 2-123 | m/z = 803.36(C₆₂H₄₅N = 804.05) | 2-124 | m/z = 834.40(C₆₄H₄₂D₅N = 835.12) |
| 2-125 | m/z = 883.33(C₆₆H₄₅NS = 884.15) | 2-126 | m/z = 651.26(C₄₉H₃₃NO = 651.81) |
| 2-127 | m/z = 641.22(C₄₇H₃₁NS = 641.83) | 2-128 | m/z = 700.29(C₅₃H₃₆N₂ = 700.89) |
| 2-129 | m/z = 677.31(C₅₂H₃₉N = 677.89) | 2-130 | m/z = 651.26(C₄₉H₃₃NO = 651.81) |
| 2-131 | m/z = 843.30(C₆₃H₄₁NS = 844.09) | 2-132 | m/z = 701.28(C₅₂H₃₅N₃ = 701.87) |
| 2-133 | m/z = 677.31(C₅₂H₃₉N = 677.89) | 2-134 | m/z = 729.28(C₅₃H₃₅N₃O = 729.88) |
| 2-135 | m/z = 912.29(C₆₄H₄₀N₄OS = 913.11) | 2-136 | m/z = 878.34(C₆₅H₄₂N₄ = 879.08) |
| 2-137 | m/z = 805.35(C₆₀H₄₃N₃ = 806.03) | 2-138 | m/z = 906.34(C₆₆H₄₂N₄O = 907.09) |
| 2-139 | m/z = 769.26(C₅₅H₃₅N₃S = 769.97) | 2-140 | m/z = 884.30(C₆₃H₄₀N₄S = 885.10) |
| 2-141 | m/z = 767.36(C₅₉H₄₅N = 768.02) | 2-142 | m/z = 815.32(C₆₂H₄₁NO = 816.02) |
| 2-143 | m/z = 829.28(C₆₂H₃₉NS = 830.06) | 2-144 | m/z = 781.35(C₅₉H₃₅D₅N₂ = 782.01) |
| 2-145 | m/z = 695.30(C₅₂H₃₈FN = 695.88) | 2-146 | m/z = 753.34(C₅₈H₄₃N = 753.99) |
| 2-147 | m/z = 803.36(C₆₂H₄₅N = 804.05) | 2-148 | m/z = 6829.37(C₆₄H₄₇N = 830.09) |
| 2-149 | m/z = 918.40(C₇₀H₅₀N₂ = 919.18) | 2-150 | m/z = 717.34(C₅₅H₄₃N = 717.96) |
| 2-151 | m/z = 717.34(C₅₅H₄₃N = 717.96) | 2-152 | m/z = 717.34(C₅₅H₄₃N = 717.96) |
| 2-153 | m/z = 717.34(C₅₅H₄₃N = 717.96) | 2-154 | m/z = 767.32(C₅₈H₄₁NO = 767.97) |
| 2-155 | m/z = 701.37(C₅₂H₄₇NO = 701.95) | 2-156 | m/z = 707.32(C₅₃H₄₁NO = 707.92) |
| 2-157 | m/z = 672.26(C₄₉H₂₈D₅NS = 672.9) | 2-158 | m/z = 824.33(C₆₁H₃₆D₅NS = 825.1) |
| 2-159 | m/z = 819.3(C₆₁H₄₁NS = 820.07) | 2-160 | m/z = 777.3(C₅₉H₃₉NO = 777.97) |
| 2-161 | m/z = 727.29(C₅₅H₃₇NO = 727.91) | 2-162 | m/z = 732.32(C₅₅H₃₂D₅NO = 732.94) |
| 2-163 | m/z = 777.3(C₅₉H₃₉NO = 777.97) | 2-164 | m/z = 743.26(C₅₅H₃₇NS = 743.97) |
| 2-165 | m/z = 727.29(C₅₅H₃₇NO = 727.91) | 2-166 | m/z = 819.3(C₆₁H₄₁NS = 820.07) |
| 2-167 | m/z = 821.35(C₆₁H₃₅D₅N₂O = 822.03) | 2-168 | m/z = 767.32(C₅₈H₄₁NO = 767.97) |
| 2-169 | m/z = 859.33(C₆₄H₄₅NS = 860.13) | 2-170 | m/z = 833.28(C₆₁H₃₉NOS = 834.05) |
| 2-171 | m/z = 825.34(C₆₁H₄₇NS = 826.11) | 2-172 | m/z = 877.37(C₆₅H₅₁NS = 878.19) |
| 2-173 | m/z = 799.42(C₅₈H₅₇NS = 800.16) | 2-174 | m/z = 803.32(C₆₁H₄₁NO = 804) |
| 2-175 | m/z = 727.29(C₅₅H₃₇NO = 727.91) | 2-176 | m/z = 651.26(C₄₉H₃₃NO = 651.81) |
| 2-177 | m/z = 667.23(C₄₉H₃₃NS = 667.87) | 2-178 | m/z = 791.28(C₅₉H₃₇NO2 = 791.95) |
| 2-179 | m/z = 827.38(C₆₁H₂₉D₁₁N₂O = 828.07) | 2-180 | m/z = 751.29(C₅₇H₃₇NO = 751.93) |
| 2-181 | m/z = 757.28(C₅₆H₃₉NS = 757.99) | 2-182 | m/z = 777.3(C₅₉H₃₉NO = 777.97) |
| 2-183 | m/z = 843.3(C₆₃H₄₁NS = 844.09) | 2-184 | m/z = 775.29(C₅₉H₃₇NO = 775.95) |
| 2-185 | m/z = 767.26(C₅₇H₃₇NS = 767.99) | 2-186 | m/z = 727.29(C₅₅H₃₇NO = 727.91) |
| 2-187 | m/z = 667.23(C₄₉H₃₃NS = 667.87) | | |
| 2-201 | m/z = 691.32(C₅₃H₄₁N = 691.92) | 2-202 | m/z = 865.37(C₆₇H₄₇N = 866.12) |
| 2-203 | m/z = 879.39(C₆₈H₄₉N = 880.15) | 2-204 | m/z = 671.36(C₅₁H₄₅N = 671.93) |
| 2-205 | m/z = 767.36(C₅₉H₄₅N = 768.02) | 2-206 | m/z = 663.38(C₅₀H₃₇D₆N = 663.94) |
| 2-207 | m/z = 820.39(C₆₃H₄₀D₅N = 821.09) | 2-208 | m/z = 759.38(C₅₈H₃₇D₆N = 760.03) |
| 2-209 | m/z = 691.32(C₅₃H₄₁N = 691.92) | 2-210 | m/z = 691.32(C₅₃H₄₁N = 691.92) |
| 2-211 | m/z = 765.42(C₅₈H₄₃D₆N = 766.07) | 2-212 | m/z = 792.36(C₆₁H₄₀D₃N = 793.04) |
| 2-213 | m/z = 932.42(C₇₂H₄₄D₅N = 933.22) | 2-214 | m/z = 738.40(C₅₆H₃₄D₉N = 739.02) |
| 2-215 | m/z = 781.37(C₆₀H₄₇N = 782.04) | 2-216 | m/z = 767.36(C₅₉H₄₅N = 768.02) |
| 2-217 | m/z = 691.32(C₅₃H₄₁N = 691.92) | 2-218 | m/z = 677.31(C₅₂H₃₉N = 677.89) |
| 2-219 | m/z = 691.32(C₅₃H₄₁N = 691.92) | 2-220 | m/z = 753.34(C₅₈H₄₃N = 753.99) |
| 2-221 | m/z = 743.36(C₅₇H₄₅N = 743.99) | 2-222 | m/z = 769.37(C₅₉H₄₇N = 770.03) |
| 2-223 | m/z = 677.31(C₅₂H₃₉N = 677.89) | 2-224 | m/z = 697.36(C₅₃H₃₅D₆N = 697.95) |
| 2-225 | m/z = 801.34(C₆₂H₄₃N = 802.03) | 2-226 | m/z = 733.37(C₅₆H₄₇N = 734.00) |
| 2-227 | m/z = 742.34(C₅₇H₃₈D₃N = 742.98) | 2-228 | m/z = 747.39(C₅₇H₄₉N = 748.03) |
| 2-229 | m/z = 879.39(C₆₈H₄₉N = 880.15) | 2-230 | m/z = 803.44(C₆₁H₄₅D₆N = 804.12) |
| 2-231 | m/z = 853.37(C₆₆H₄₇N = 854.11) | 2-232 | m/z = 763.40(C₅₈H₃₃D₁₀N = 764.05) |
| 2-233 | m/z = 753.34(C₅₈H₄₃N = 753.99) | 2-234 | m/z = 891.39(C₆₉H₄₉N = 892.16) |
| 2-235 | m/z = 807.39(C₆₂H₄₉N = 808.08) | 2-236 | m/z = 757.37(C₅₈H₄₇N = 758.02) |
| 2-237 | m/z = 835.41(C₆₄H₄₁D₆N = 836.12) | 2-238 | m/z = 891.39(C₆₉H₄₉N = 892.16) |
| 2-239 | m/z = 784.39(C₆₀H₄₄D₃N = 785.06) | 2-240 | m/z = 763.40(C₅₈H₃₃D₁₀N = 764.05) |

Although the above has been described with respect to exemplary synthetic examples of the compounds of the present invention, they are all based on the Buchwald-Hartwig cross coupling reaction, Miyaura boration reaction, Suzuki cross-coupling reaction, Intramolecular acid-induced cyclization reaction (J. mater. Chem. 1999, 9, 2095), Pd(II)-catalyzed oxidative cyclization reaction (Org. Lett. 2011, 13, 5504) and the PPh₃-mediated reductive cyclization reaction (J. Org. Chem. 2005, 70, 5014), and it will be easily understood by those skilled in the art that the above reaction proceeds even if a substituent other than the substituent specified in the specific synthetic examples is combined.

### Evaluation of Manufacture of Organic Electronic Element

### Example 5) Manufacture and Evaluation of Blue Organic Light Emitting Diode

First, on an ITO layer (anode) formed on a glass substrate, N¹-(naphthalen-2-yl)-N⁴,N⁴-bis(4-(naphthalen-2-yl(phenyl)amino)phenyl)-N¹-phenylbenzene-1,4-diamine (hereinafter will be abbreviated as 2-TNATA) was vacuum-deposited to form a hole injection layer with a thickness of 60 nm. Subsequently, and on the layer, 4,4-bis[N-(1-naphthyl)-N-phenylamino]biphenyl (hereinafter will be abbreviated as NPD) was vacuum-deposited to a thickness of 60 nm to form a hole transport layer. Subsequently, the inventive compound was vacuum-deposited as an emitting auxiliary layer material to a thickness of 20 nm to form an emitting auxiliary layer. After forming the emitting auxiliary layer, on the emitting auxiliary layer, 9,10-di(naphthalen-2-yl)anthracene is used as a host, and BD-052X (Idemitsu kosan) is used as dopant in a ratio of 96:4, therefore an emitting layer with a thickness of 30 nm was deposited on the emitting auxiliary layer. (1,1′-bisphenyl)-4-olato)bis(2-methyl-8-quinolinolato)aluminum (hereinafter abbreviated as BAlq) was vacuum deposited as a hole blocking layer to a thickness of 10 nm, and Tris(8-quinolinol) aluminum (hereinafter abbreviated as Alq3) was deposited to a thickness of 40 nm as an electron transport layer. After that, an alkali metal halide, LiF was vacuum deposited as an electron injection layer to a thickness of 0.2 nm, and Al was deposited to a thickness of 150 nm to form a cathode to manufacture an OLED.

To the OLEDs which were manufactured by examples and comparative examples, a forward bias direct current voltage was applied, and electroluminescent (EL) properties were measured using PR-650 of Photoresearch Co., and T95 life was measured using a life measuring apparatus manufactured by McScience Inc. with a reference luminance of 5000 cd/m². In the following table, the manufacture of a device and the results of evaluation are shown. To the OLEDs which were manufactured by examples and comparative examples, a forward bias direct current voltage was applied, and electroluminescent (EL) properties were measured using PR-650 of Photoresearch Co., and T95 life was measured using a life measuring apparatus manufactured by McScience Inc. with a reference luminance of 5000 cd/m². In the following table, the manufacture of a device and the results of evaluation are shown.

### Comparative Examples 35˜37

An OLED was prepared in the same manner as in Example 5 except that the emitting auxiliary layer was not used and Comparative Compound F, Comparative Compound G and Invention Compound 2-81 were used as the hole transport layer material.

### Comparative Example 38

An OLED was prepared in the same manner as in Example 5 except that the emitting auxiliary layer was not used.

### Comparative Examples 39˜40

An OLED was prepared in the same manner as in Example 5 except that the Comparative Compound F or Comparative Compound G were used as the emitting auxiliary layer material.

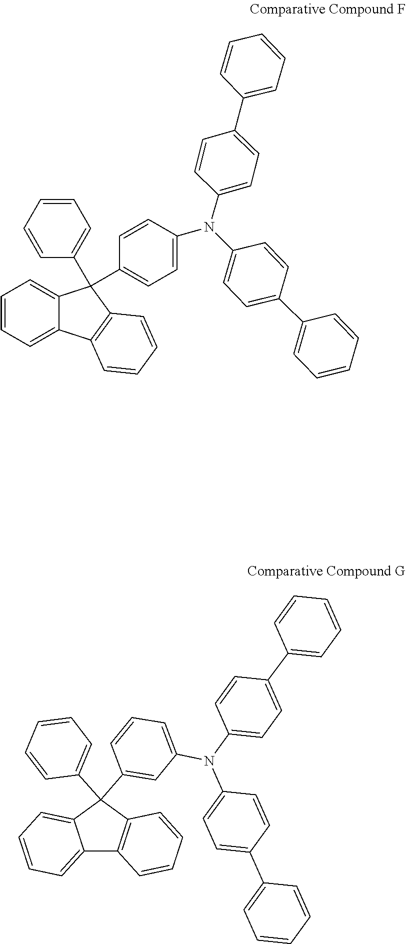 [SMILES: c1ccc(-c2ccc(N(c3ccc(-c4ccccc4)cc3)c3ccc(C4(c5ccccc5)c5ccccc5-c5ccccc54)cc3)cc2)cc1 ; c1ccc(-c2ccc(N(c3ccc(-c4ccccc4)cc3)c3cccc(C4(c5ccccc5)c5ccccc5-c5ccccc54)c3)cc2)cc1]

TABLE 11

| | | Emitting | | | | | |
|---|---|---|---|---|---|---|---|
| | | auxiliary | | Current | | | |
| | Hole transport | layer | | density | brightness | Efficiency | |
| | compound | compound | voltage | (mA/cm²) | (cd/m²) | (cd/A) | T(95) |
| comparative | comparative | — | 6.8 | 14.3 | 500.0 | 3.5 | 70.4 |
| example (35) | compound F | | | | | | |
| comparative | comparative | | 7.0 | 15.2 | 500.0 | 3.3 | 69.6 |
| example (36) | compound G | | | | | | |
| comparative | 2-81 | | 6.9 | 13.5 | 500.0 | 3.7 | 71.2 |
| example (37) | | | | | | | |
| comparative | NPB | — | 7.3 | 17.9 | 500.0 | 2.8 | 65.3 |
| example (38) | | | | | | | |
| comparative | | comparative | 6.8 | 11.1 | 500.0 | 4.5 | 80.4 |
| example (39) | | compound F | | | | | |
| comparative | | comparative | 6.5 | 10.2 | 500.0 | 4.9 | 81.7 |
| example (40) | | compound G | | | | | |
| example(57) | | 2-81 | 6.3 | 9.1 | 500.0 | 5.5 | 94.9 |
| example(58) | | 2-83 | 6.2 | 8.6 | 500.0 | 5.8 | 97.0 |
| example(59) | | 2-85 | 6.1 | 9.2 | 500.0 | 5.4 | 93.2 |
| example(60) | | 2-86 | 6.2 | 8.9 | 500.0 | 5.6 | 96.9 |
| example(61) | | 2-89 | 6.1 | 9.3 | 500.0 | 5.4 | 94.1 |
| example(62) | | 2-107 | 6.1 | 9.3 | 500.0 | 5.4 | 94.8 |
| example(63) | | 2-122 | 6.0 | 9.1 | 500.0 | 5.5 | 94.7 |
| example(64) | | 2-127 | 6.2 | 8.8 | 500.0 | 5.7 | 96.0 |
| example(65) | | 2-128 | 6.1 | 8.7 | 500.0 | 5.7 | 95.6 |
| example(66) | | 2-133 | 6.1 | 9.4 | 500.0 | 5.3 | 94.3 |
| example(67) | | 2-150 | 6.2 | 9.2 | 500.0 | 5.4 | 93.6 |
| example(68) | | 2-151 | 6.0 | 9.4 | 500.0 | 5.3 | 94.1 |
| example(69) | | 2-152 | 6.0 | 9.1 | 500.0 | 5.5 | 93.7 |
| example(70) | | 2-153 | 6.1 | 9.4 | 500.0 | 5.3 | 93.2 |

### Example 6) Manufacture and Evaluation of Green Organic Light Emitting Diode

First, on an ITO layer (anode) formed on a glass substrate, N¹-(naphthalen-2-yl)-N⁴,N⁴-bis(4-(naphthalen-2-yl(phenyl)amino)phenyl)-N¹-phenylbenzene-1,4-diamine (hereinafter will be abbreviated as 2-TNATA) was vacuum-deposited to form a hole injection layer with a thickness of 60 nm. Subsequently, and on the layer, 4,4-bis[N-(1-naphthyl)-N-phenylamino]biphenyl (hereinafter will be abbreviated as NPD) was vacuum-deposited as a hole transport compound to a thickness of 60 nm to form a hole transport layer. Subsequently, the inventive compound represented by Formula (30) was vacuum-deposited as an emitting auxiliary layer material to a thickness of 20 nm to form an emitting auxiliary layer. After forming the emitting auxiliary layer, on the emitting auxiliary layer, 4,4′-di(9H-carbazol-9-yl)-1,1′-biphenyl is used as a host, and Ir(ppy)3[tris(2-phenylpyridine)-iridium] is used as dopant in a ratio of 95:5, and an emitting layer with a thickness of 30 nm was deposited. (1,1′-bisphenyl)-4-olato)bis(2-methyl-8-quinolinolato)aluminum (hereinafter abbreviated as BAlq) was vacuum deposited as a hole blocking layer to a thickness of 10 nm, and Tris(8-quinolinol)aluminum (hereinafter abbreviated as Alq3) was deposited to a thickness of 40 nm as an electron transport layer. After that, an alkali metal halide, LiF was vacuum deposited as an electron injection layer to a thickness of 0.2 nm, and Al was deposited to a thickness of 150 nm to form a cathode to manufacture an OLED.

To the OLEDs which were manufactured by examples and comparative examples, a forward bias direct current voltage was applied, and electroluminescent (EL) properties were measured using PR-650 of Photoresearch Co., and T95 life was measured using a life measuring apparatus manufactured by McScience Inc. with a reference luminance of 5000 cd/m². In the following table, the manufacture of a device and the results of evaluation are shown.

### Comparative Examples 41˜42

An OLED was prepared in the same manner as in Example 6 except that Comparative Compound F or Comparative Compound G were used as the emitting auxiliary layer material.

TABLE 12

| | | | Current | | | | | |
|---|---|---|---|---|---|---|---|---|
| | | | density | brightness | Efficiency | | CIE | |
| | compound | voltage | (mA/cm²) | (cd/m²) | (cd/A) | T(95) | x | y |
| comparative | comparative | 7.3 | 17.4 | 5000.0 | 28.7 | 80.4 | 0.30 | 0.61 |
| example(41) | compound F | | | | | | | |
| comparative | comparative | 7.4 | 19.9 | 5000.0 | 25.1 | 80.9 | 0.33 | 0.64 |
| example(42) | compound G | | | | | | | |
| example(71) | 2-81 | 5.6 | 11.0 | 5000.0 | 45.4 | 120.3 | 0.31 | 0.64 |
| example(72) | 2-83 | 6.3 | 14.2 | 5000.0 | 35.1 | 104.3 | 0.31 | 0.64 |
| example(73) | 2-85 | 5.8 | 13.8 | 5000.0 | 36.2 | 110.1 | 0.34 | 0.61 |
| example(74) | 2-86 | 6.4 | 14.3 | 5000.0 | 35.0 | 103.6 | 0.33 | 0.63 |
| example(75) | 2-89 | 6.0 | 14.1 | 5000.0 | 35.5 | 113.0 | 0.33 | 0.65 |
| example(76) | 2-107 | 5.8 | 11.3 | 5000.0 | 44.3 | 112.6 | 0.32 | 0.65 |
| example(77) | 2-122 | 5.9 | 11.4 | 5000.0 | 43.8 | 112.3 | 0.34 | 0.63 |
| example(78) | 2-127 | 6.4 | 12.6 | 5000.0 | 39.8 | 104.2 | 0.33 | 0.65 |
| example(79) | 2-128 | 6.3 | 12.8 | 5000.0 | 39.2 | 102.5 | 0.32 | 0.65 |
| example(80) | 2-133 | 5.9 | 12.5 | 5000.0 | 39.9 | 111.2 | 0.32 | 0.65 |
| example(81) | 2-150 | 5.4 | 11.4 | 5000.0 | 43.8 | 114.8 | 0.31 | 0.64 |
| example(82) | 2-151 | 5.2 | 11.2 | 5000.0 | 44.8 | 111.5 | 0.30 | 0.63 |
| example(83) | 2-152 | 5.2 | 11.6 | 5000.0 | 43.1 | 112.1 | 0.31 | 0.63 |
| example(84) | 2-153 | 5.3 | 11.2 | 5000.0 | 44.8 | 114.4 | 0.30 | 0.62 |

As can be seen from the results of Table 11 to 12, when OLED was manufactured by using the material for an organic electroluminescence device of the present invention as an emitting auxiliary layer material, the driving voltage of the organic electroluminescent device can be lowered and the luminous efficiency and lifetime can be remarkably improved as compared with the comparative example not using the material for the emitting auxiliary layer or using the comparative compound For the comparative compound G.

Table 11 shows the results of the production of a blue organic light emitting device. It can be confirmed that excellent results are obtained when the compound of the present invention is used as an emitting auxiliary layer. The results of Comparative Example 39 or Comparative Example 40 and Examples 57 to 70 show that compounds of the present invention substituted with specific substituents such as DBT, DBF, Cz, and Fluorene are remarkably superior to the comparative compounds substituted with the general aryl group even though the mother compound is similar. That is, when specific substituents such as DBT, DBF, Cz, and Fluorene are introduced, the refractive index, the Tg, and the energy level of the compound (HOMO, LUMO, T1, etc.) become significantly different, and this difference in physical properties is a major factor in improving the device performance during device deposition (for example, such as an energy balance), such that different device results can be derived.

Table 12 shows the results of the production of a green organic light emitting device. When the compound of the present invention was used as an emitting auxiliary layer, the results were significantly superior to the comparative compounds. This is also the effect of certain substituents such as DBT, DBF, Cz, and Fluorene, and a specific feature is that the superiority of the green auxiliary layer is significantly improved than the blue auxiliary layer. Further, in the case of the blue auxiliary layer, DBT and DBF substituted compounds showed the most excellent properties, but the results of the green auxiliary layer showed the best results with the Fluorene substituted compounds. This suggests that even if the emitting auxiliary layer compound is the same, the properties required depending on the color of the emitting layer are different, so that a result which can not be deduced by those skilled in the art can be obtained.

### Example 7) Manufacture and Evaluation of of Tandem OLED

When the organic electronic element according to the present specification is a top emission type and the first electrode is formed on the substrate before the formation of the organic material layer and the second electrode, not only a transparent material but also an opaque material with excellent light reflectivity can be used as the first electrode material.

When the organic electronic element according to the present specification is a bottom emission type and the first electrode is formed on the substrate before the formation of the organic material layer and the second electrode, a transparent material should be used as the first electrode material, or an opaque material should be formed into a thin film so as to become transparent.

This embodiment presents the following examples by manufacturing a top emission Tandem OLED, but the embodiments of the present invention are not limited thereto. A Tandem OLED according to one embodiment of the present invention is manufactured by connecting a plurality of stacks through charge generation layers.

A Tandem OLED according to one embodiment of the present invention uses the same compound in the emitting layer and electron transport layer of each of the 2 stacks, but is not limited thereto.

### [Example 85] Tandem OLED with 2 Stacks Connected

A Tandem OLED with 2 stacks connected was fabricated with the structure of the first electrode/hole transport region/emitting layer/electron transport region/charge generation region/hole transport region/emitting layer/electron transport region/electron injection layer/second electrode.

Specifically, N¹-(naphthalen-2-yl)-N⁴, N⁴-bis(4-(naphthalen-2-yl(phenyl)amino)phenyl)-N¹-phenylbenzene-1,4-diamine (hereinafter, abbreviated as 2-TNATA) and HATCN were used on an ITO layer (first electrode) formed on a glass substrate, and HATCN was doped in a weight ratio of 90:10 to form a 60 nm thick hole injection layer. N,N′-bis(1-naphthalenyl)-N,N′-bis-phenyl-(1,1′-biphenyl)-4,4′-diamine (hereinafter, abbreviated as NPB) was vacuum-deposited on the hole injection layer to a thickness of 145 nm to form a hole transport layer.

Next, 4,4′-Bis(2,2-diphenylethenyl)-1,1′-biphenyl (hereinafter, abbreviated as DPVBi) was used as the host material of the emitting layer on the hole transport layer, and 4,4′-bis(9-ethyl-3-carbazovinylene)-1,1′-biphenyl (hereinafter abbreviated as BCzVBi) was used as the dopant material, and the dopants were doped at a weight ratio of 95:5 to form an emitting layer having a thickness of 115 nm.

Next, on the light-emitting layer, Tris(8-hydroxyquinolinato)aluminium (hereinafter, abbreviated as Alq₃) was vacuum-deposited to a thickness of 90 nm to form an electron transport layer. (hereinafter, the first stack)

Next, to connect 2 stacks, Bathophenanthroline (hereinafter abbreviated as Bphen) and Li were used, and Li was doped in a weight ratio of 97:3 to form an n-type charge generation layer with a thickness of 50 nm, and on the n-type charge generation layer, the compound 2-89 of the present invention and HATCN were used, and HATCN was doped in a weight ratio of 90:10 to form a p-type charge generation layer with a thickness of 65 nm.

On the p-type charge generation layer, a hole transport layer, an emitting layer, and an electron transport layer identical to those formed in the first stack were sequentially formed. (Hereinafter, the second stack)

Afterwards, lithium fluoride (hereinafter abbreviated as LiF) was vacuum-deposited to a thickness of 6 nm as an electron injection layer, and then Ag:Mg was deposited to a thickness of 150 nm to form a second electrode, thereby manufacturing a Tandem OLED.

### [Example 86] to [Example 101]

An organic light emitting device was manufactured in the same manner as in Example 1, except that the compounds listed in Table 3 were used as n-type charge generation layer and p-type charge generation layer materials.

### [Comparative Example 43] to [Comparative Example 44]

An organic light emitting device was manufactured in the same manner as in Example 85, except that the comparative compound F or comparative compound G were used as n-type charge generation layer and p-type charge generation layer materials.

The electroluminescence (EL) characteristics were measured using PR-650 from Photoresearch by applying a forward bias DC voltage to the organic electroluminescence devices manufactured by the examples and comparative examples manufactured in this way. As a result of the measurement, the T95 lifespan was measured using a lifespan measuring device manufactured by Max Science at a standard brightness of 1500 cd/m². The Table 13 shows the results of the device fabrication and evaluation.

The measuring apparatus can evaluate the performance of new materials compared to comparative compounds under identical conditions, without being affected by possible daily fluctuations in deposition rate, vacuum quality or other parameters.

During the evaluation, one batch contains 4 identically prepared OLEDs including a comparative compound, and the performance of a total of 12 OLEDs is evaluated in 3 batches, so the value of the experimental results obtained in this way indicates statistical significance.

TABLE 13

| | | | Current Density | Luminance | Efficiency | |
|---|---|---|---|---|---|---|
| | compound | Driving voltage | (mA/cm²) | (cd/m²) | (cd/A) | T(95) |
| comparative | comparative | 13.9 | 17.0 | 1500.0 | 8.8 | 54.7 |
| example(43) | compound F | | | | | |
| comparative | comparative | 13.1 | 16.0 | 1500.0 | 9.4 | 60.1 |
| example(44) | compound G | | | | | |
| example(85) | 2-89 | 8.0 | 12.3 | 1500.0 | 12.2 | 71.2 |
| example(86) | 2-107 | 7.9 | 11.5 | 1500.0 | 13.0 | 81.1 |
| example(87) | 2-111 | 7.9 | 11.5 | 1500.0 | 13.1 | 80.5 |
| example(88) | 2-133 | 8.0 | 12.1 | 1500.0 | 12.4 | 72.6 |
| example(89) | 2-201 | 7.6 | 10.1 | 1500.0 | 14.8 | 85.9 |
| example(90) | 2-203 | 7.6 | 10.2 | 1500.0 | 14.7 | 86.7 |
| example(91) | 2-204 | 7.6 | 10.1 | 1500.0 | 14.8 | 86.1 |
| example(92) | 2-212 | 7.7 | 10.3 | 1500.0 | 14.5 | 84.0 |
| example(93) | 2-215 | 7.6 | 10.3 | 1500.0 | 14.6 | 84.6 |
| example(94) | 2-217 | 7.3 | 9.6 | 1500.0 | 15.6 | 90.8 |
| example(95) | 2-218 | 7.4 | 9.7 | 1500.0 | 15.5 | 89.8 |
| example(96) | 2-222 | 7.5 | 9.9 | 1500.0 | 15.2 | 89.1 |
| example(97) | 2-224 | 7.4 | 9.6 | 1500.0 | 15.7 | 91.1 |
| example(98) | 2-226 | 7.5 | 9.8 | 1500.0 | 15.3 | 89.5 |
| example(99) | 2-231 | 7.5 | 10.0 | 1500.0 | 15.0 | 88.7 |
| example(100) | 2-236 | 7.7 | 10.5 | 1500.0 | 14.3 | 83.1 |
| example(101) | 2-240 | 7.8 | 10.6 | 1500.0 | 14.1 | 82.9 |

As can be seen from the results in Table 13, when a 2-stack Tandem OLED is manufactured using the compound of the present invention as a p-type charge generation layer material, the operating voltage, efficiency, and lifespan of the organic electronic element can be improved compared to the comparative example using the comparative compound F or comparative compound G having a similar basic skeleton to the compound of the present invention.

In the case of the p-type charge generation layer, the ability to transfer holes from the p-type charge generation layer to the hole transport layer of the second stack and the ability to quickly move electrons to the n-type charge generation layer are required, and the charge separation ability of the compound of the present invention is thought to be superior to that of comparative compound F or comparative compound G.

Additionally, when examining the compounds of the present invention, it was found that when a compound represented by Formula (32) was applied to a device, the performance of the device was superior to that of a compound having a bonding framework of Formula (31) or Formula (33). In particular, when a substituent other than H was further substituted at positions R24 and R25, the performance of the device showed superior results than when other compounds were applied.

In other words, as can be seen from the results in Table 13, it can be confirmed that a compound satisfying both the structural features and composition disclosed in the present invention exhibits a remarkable effect in an organic electronic element, compared to comparative compounds F and G, which have a structurally similar composition to the compound of the present invention, and this shows that the compound of the present invention, which satisfies all specific configurations, exhibits a remarkable effect compared to other comparative compounds not described in this specification.

These results show that even in compounds with similar molecular components, depending on the type and substitution position of the substituent, the properties of compounds such as the hole characteristics, light efficiency characteristics, energy level, hole injection and mobility characteristics, charge balance of holes and electrons, volume density, and intermolecular distance of the molecule may vary significantly enough to be difficult to predict, additionally, it suggests that rather than one configuration affecting the overall results of the element, the performance of the element may vary due to complex factors.

Although exemplary embodiments of the present invention have been described for illustrative purposes, those skilled in the art will appreciate that various modifications, additions and substitutions are possible, without departing from the scope and spirit of the invention as disclosed in the accompanying claims. Therefore, the embodiment disclosed in the present invention is intended to illustrate the scope of the technical idea of the present invention, and the scope of the present invention is not limited by the embodiment. The scope of the present invention shall be construed on the basis of the accompanying claims, and it shall be construed that all of the technical ideas included within the scope equivalent to the claims belong to the present invention.

What is claimed is:

1. A compound of Formula (32): 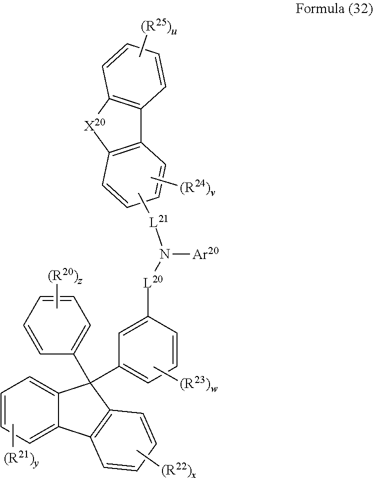
wherein:
1) R²⁰, R²¹, R²², R²³, R²⁴ and R²⁵ are each independently selected from the group consisting of hydrogen deuterium, a C₆-C₃₀ aryl group and a C₁-C₂₀ alkyl group, and a plurality of R²⁰, a plurality of R²¹, a plurality of R²², a plurality of R²³, and a plurality of R²⁴ are not bonded to each other to form a ring,
2) v is an integer of 0 to 3, u, w, x and y are each independently an integer of 0 to 4, z is an integer of 0 to 5,
3) L²⁰ and L²¹ are each a single bond,
4) Ar²⁰ is a C₆-C₃₀ aryl group,
5) X²⁰ is CR′R″, and
6) R′ and R″ are each independently selected from the group of a C₁-C₃₀ alkyl group; a C₆-C₃₀ aryl group; and R′ and R″ may be bonded to each other to form a spiro,
wherein the aryl group and alkyl group may be each substituted with one or more substituent(s) selected from the group consisting of deuterium, halogen, a C₁-C₂₀ alkyl group, a C₂-C₂₀ alkenyl group, a C₆-C₂₀ aryl group, a C₆-C₂₀ aryl group substituted with deuterium, a fluorenyl group, a C₂-C₂₀ heterocyclic group, and a C₃-C₂₀ cycloalkyl group, and wherein the substituent(s) may be substituted with one or more deuterium.

2. The compound of claim 1, wherein Formula 32 is represented by Formula 38 or Formula 39:

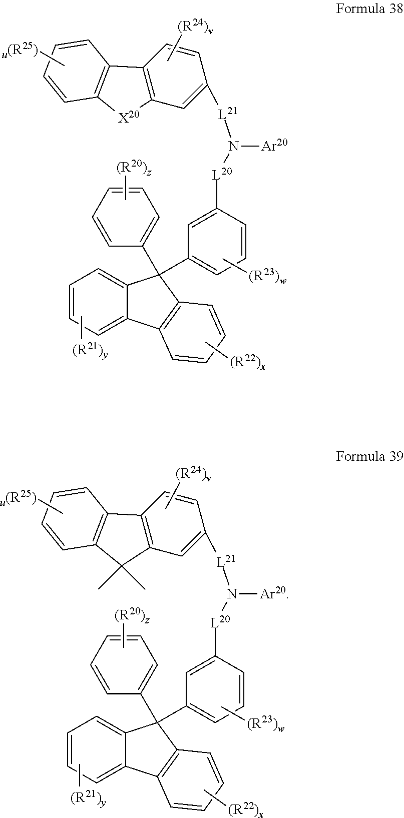

3. The compound of claim 1, wherein u+v+w+x+y+z≥1 and at least one of R²⁰ to R²⁵ is an alkyl group or an aryl group.

4. The compound of claim 1, wherein Ar²⁰ is represented by any of Formulas Ar-1 to Ar-8: 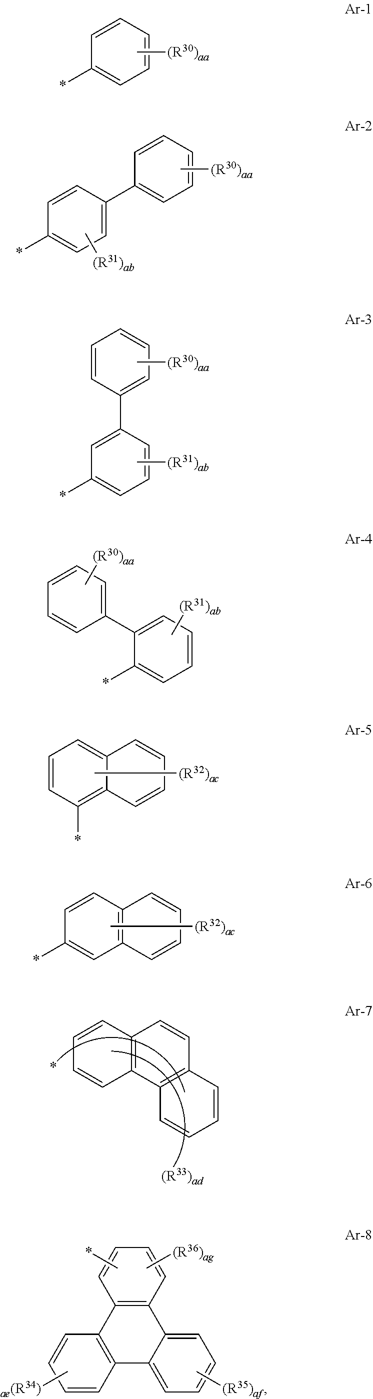
wherein:
1) R³⁰, R³¹, R³², R³³, R³⁴, R³⁵ and R³⁶ are each independently selected from the group consisting of hydrogen, deuterium, a C₁-C₃₀ alkyl group, and a C₆-C₂₀ aryl group, and a plurality of R³⁰, a plurality of R³¹, a plurality of R³², a plurality of R³³, a plurality of R³⁴, a plurality of R³⁵, a plurality of R³⁶, a plurality of R³⁷, or a plurality of R³⁸, each in the plurality being the same as or different from each other, can be bonded to each other to form an aromatic or a heteroaromatic ring, and
2) aa is an integer of 0 to 5; ab is an integer of 0 to 4; ac is an integer of 0 to 7; ad is an integer of 0 to 9; ae, af and ai are each independently an integer of 0 to 4;
ag and ah are each independently an integer of 0 to 3.

5. The compound of claim 1, wherein the compound of Formula 32 is any of the following compounds.

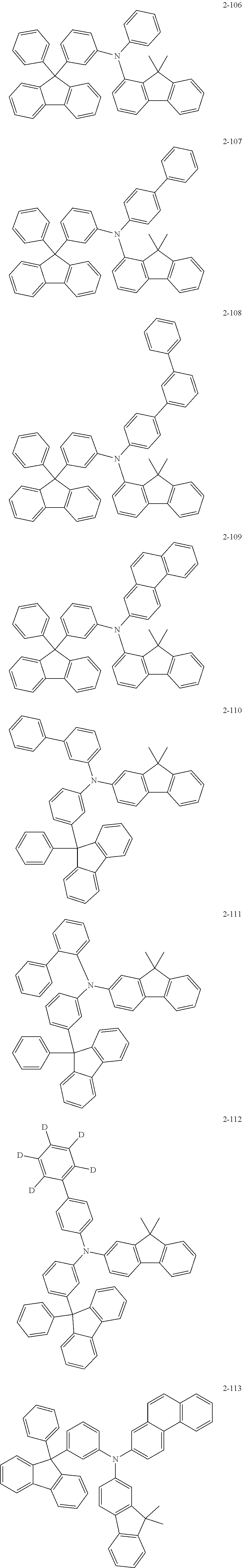

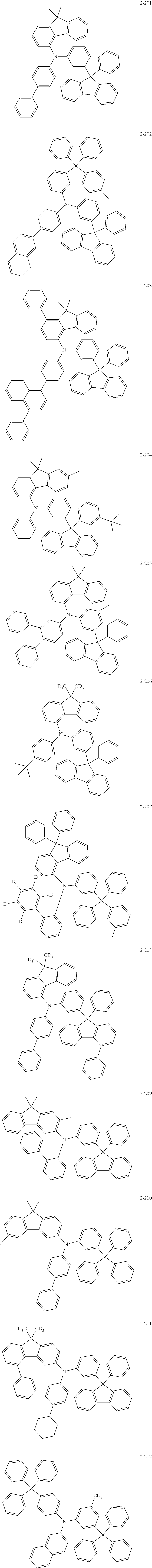

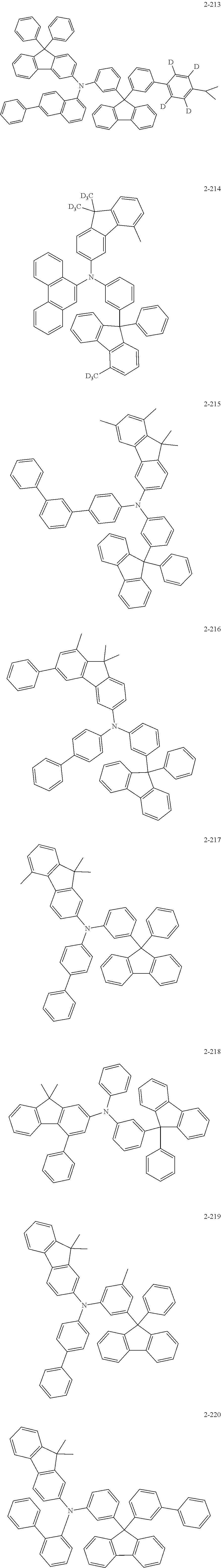

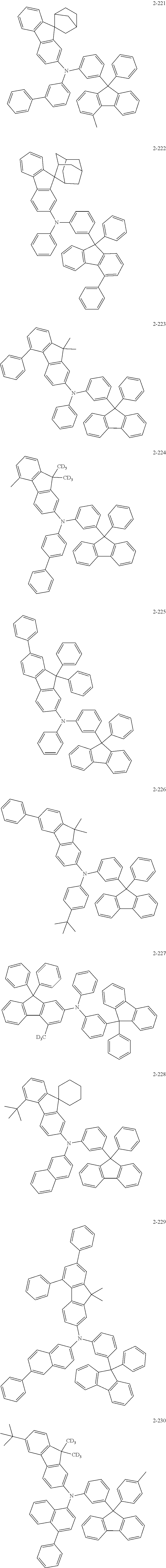

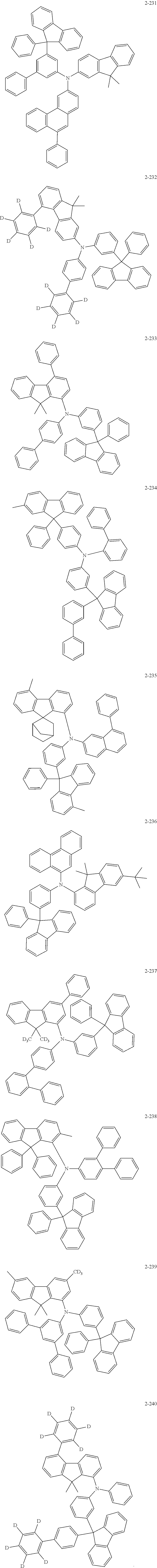

6. An organic electronic element comprising an anode; a cathode; and an organic layer formed between the anode and the cathode, wherein the organic layer comprises a compound of Formula 32 of claim 1.

7. The organic electronic element of claim 6, wherein the organic layer comprises at least 2 or more stacks of layers.

8. The organic electronic element of claim 7, wherein a charge generation region is included between the 2 or more stacks.

9. The organic electronic element of claim 8, wherein the charge generation region comprises a n-type charge generation layer and a p-type charge generation layer.

10. The organic electronic element of claim 9, wherein the p-type charge generation layer comprises a compound of Formula 32.
